(19) United States
(12) Patent Application Publication
(10) Pub. No.: US 2004/0192729 A1
(43) Pub. Date: Sep. 30, 2004

(54) Carboxylic acids and the esters thereof, pharmaceutical compositions thereto and processes for the preparation thereof

(75) Inventors: Klaus Rudolf, Warthausen (DE); Stephan Georg Mueller, Warthausen (DE); Marcus Schindler, Biberach (DE); Dirk Stenkamp, Biberach (DE); Eckhart Bauer, Biberach (DE); Kai Gerlach, Ulm (DE); Rudolf Hurnaus, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/755,593

(22) Filed: Jan. 12, 2004

Related U.S. Application Data
(60) Provisional application No. 60/443,492, filed on Jan. 29, 2003.

(30) Foreign Application Priority Data
Jan. 14, 2003 (DE) 10300973.6

(51) Int. Cl. C07D 417/14; C 07D 4 1/14; A61K 31/4545

(52) U.S. Cl. 514/316; 546/187

(57) ABSTRACT

The present invention relates to carboxylic acids and esters of general formula

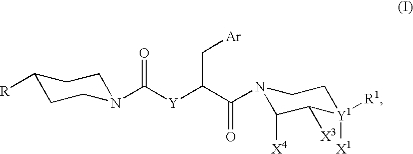

wherein Ar, R, R¹, X¹, X³, X⁴, Y and Y¹ are defined as in claim 1, the tautomers, the diastereomers, the enantiomers, the mixtures thereof and the salts thereof, particularly the physiologically acceptable salts thereof with inorganic or organic acids or bases, pharmaceutical compositions containing these compounds, the use thereof and processes for the preparation thereof, as well as the use thereof for the production and purification of antibodies and as labelled compounds in RIA and ELISA assays and as diagnostic or analytical aids in neurotransmitter research.

### RELATED APPLICATIONS

The Priority benefit of DE 103 00 973.6, filed on Jan. 14, 2003 and U.S. Provisional Application No. 60/443,492, filed on Jan. 29, 2003 are hereby claimed, both of which are incorporated by reference herein.

### DETAILED DESCRIPTION OF THE INVENTION

The present invention relates to new carboxylic acids and the esters thereof of general formula

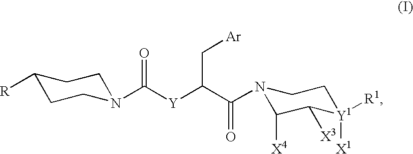

the tautomers, the diastereomers, the enantiomers, the mixtures thereof and the salts thereof, particularly the physiologically acceptable salts thereof with inorganic or organic acids or bases, pharmaceutical compositions containing these compounds, the use thereof and processes for the preparation thereof.

In the above general formula I

R denotes a monounsaturated 5- to 7-membered diaza, triaza or S,S-dioxido-thiadiaza heterocycle,

while the above-mentioned heterocycles are linked via a nitrogen atom and

are characterised by a carbonyl group or sulphonyl group each flanked by two nitrogen atoms,

may be substituted at one or at two carbon atoms by an alkyl, phenyl, pyridinyl, thienyl or 1,3-thiazolyl group, while the substituents may be identical or different,

and the double bond of one of the above-mentioned unsaturated heterocycles may be fused to a benzene, pyridine or quinoline ring,

while the phenyl, pyridinyl, thienyl, or 1,3-thiazolyl groups contained in R as well as benzo-, pyrido- and quinolino-fused heterocycles in the carbon skeleton may additionally be mono-, di- or trisubstituted by fluorine, chlorine or bromine atoms, by alkyl, alkoxy, nitro, alkylthio, alkylsulphinyl, alkylsulphonyl, alkylsulphonylamino, phenyl, trifluoromethyl, alkoxycarbonyl, carboxy, dialkylamino, hydroxy, amino, acetylamino, propionylamino, aminocarbonyl, alkylaminocarbonyl, dialkylaminocarbonyl, methylenedioxy, aminocarbonylamino, alkanoyl, cyano, trifluoromethoxy, trifluoromethylthio, trifluoromethylsulphinyl or trifluoromethylsulphonyl groups, while the substituents may be identical or different,

Ar denotes a phenyl, 1-naphthyl, 2-naphthyl, tetrahydro-1-naphthyl, tetrahydro-2-naphthyl, 1H-indol-3-yl, 1-methyl-1H-indol-3-yl, 1-formyl-1H-indol-3-yl, 4-imidazolyl, 1-methyl-4-imidazolyl, 2-thienyl, 3-thienyl, thiazolyl, 1H-indazol-3-yl, 1-methyl-1H-indazol-3-yl, benzo[b]furyl, 2,3-dihydrobenzo[b]furyl, benzo[b]thienyl, pyridinyl, quinolinyl or isoquinolinyl group,

while the above-mentioned aromatic and heteroaromatic groups may additionally be mono-, di- or trisubstituted in the carbon skeleton by fluorine, chlorine or bromine atoms, by alkyl groups, C₃₋₈-cycloalkyl groups, phenylalkyl groups, alkenyl, alkoxy, phenyl, phenylalkoxy, trifluoromethyl, alkoxycarbonyl, carboxy, dialkylamino, nitro, hydroxy, amino, alkylamino, acetylamino, propionylamino, methylsulphonyloxy, aminocarbonyl, alkylaminocarbonyl, dialkylaminocarbonyl, alkanoyl, cyano, trifluoromethoxy, trifluoromethylthio, trifluoromethylsulphinyl or trifluoromethylsulphonyl groups and the substituents may be identical or different,

Y denotes the methylene or the —NH— group,

Y¹ denotes the carbon or the nitrogen atom,

X¹ denotes the pair of free electrons, if Y¹ denotes the nitrogen atom, or, if Y¹ is the carbon atom, denotes a hydrogen atom or a carboxylic acid group optionally esterified with a lower aliphatic alcohol,

X³ and X⁴ in each case denote the hydrogen atom or the carboxylic acid group optionally esterified with a lower aliphatic alcohol,

with the proviso that at least one but also not more than one of the groups X¹, X², X³ or X⁴ contains an optionally esterified carboxylic acid function, and

R¹ denotes a group of general formula

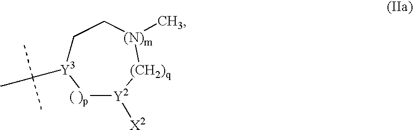

wherein

Y² denotes the carbon or, if m assumes the value 0, also the nitrogen atom,

Y³, which is always different from Y¹, denotes the carbon or nitrogen atom,

X² denotes a group of general formula

CH₂CO₂R², (III)

wherein

R² denotes the hydrogen atom or a C₁₋₅-alkyl group,

or, if Y² is the carbon atom, it may also denote the hydrogen atom or the carboxylic acid group optionally esterified with a lower aliphatic alcohol,

m denotes the numbers 0 or 1,

p denotes the numbers 0, 1, 2 or 3 and

q denotes the numbers 0, 1 or 2,

while the sum of m, p and q may assume the values 1, 2 or 3,

or one of the groups (IIb), (IIc) or (IId)

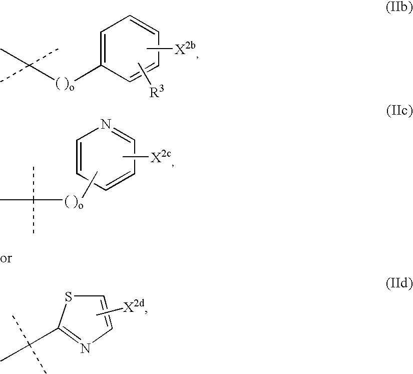

wherein

X^{2b}, X^{2c} and X^{2d} each denote the hydrogen atom or a carboxylic acid group optionally esterified with a lower aliphatic alcohol,

o denotes the numbers 0, 1, 2 or 3 and

R³ denotes the hydrogen atom, the fluorine, chlorine or bromine atom, an alkyl, alkoxy, nitro, trifluoromethyl, hydroxy, amino, acetylamino, aminocarbonyl, acetyl or cyano group,

while, unless otherwise stated, the above-mentioned alkyl groups or the alkyl groups contained in the above-mentioned groups contain 1 to 5 carbon atoms and may be straight-chain or branched.

The present invention relates to racemates, if the compounds of general formula I have only one chiral element. The application also includes, however, the individual diastereomeric pairs of antipodes or the mixtures thereof which are obtained when there is more than one chiral element in the compounds of general formula 1, as well as the individual optically active enantiomers of which the above-mentioned racemates are composed.

The compounds of general formula I have valuable pharmacological properties, which are based on their selective CGRP-antagonistic properties. The invention further relates to pharmaceutical compositions containing these compounds, the use thereof and the preparation thereof.

Preferred compounds of the above general formula I are those wherein

R denotes a monounsaturated 5- to 7-membered diaza, triaza or S,S-dioxido-thiadiaza heterocycle,

while the above-mentioned heterocycles are linked via a nitrogen atom and

are characterised by a carbonyl group or sulphonyl group in each case flanked by two nitrogen atoms,

may be substituted at a carbon atom by a phenyl, pyridinyl, thienyl or 1,3-thiazolyl group,

and the double bond of one of the above-mentioned unsaturated heterocycles may be fused to a benzene, pyridine or quinoline ring,

while the phenyl, pyridinyl, thienyl, or 1,3-thiazolyl groups contained in R as well as benzo-, pyrido- and quinolino-fused heterocycles in the carbon skeleton may additionally be mono-, di- or trisubstituted by fluorine, chlorine or bromine atoms, by alkyl, alkoxy, trifluoromethyl, amino, cyano or acetylamino groups, while the substituents may be identical or different,

Ar denotes a phenyl, 1-naphthyl, 2-naphthyl, 1,2,3,4-tetrahydro-1-naphthyl or 2,3-dihydrobenzo[b]fur-5-yl group,

while the above-mentioned aromatic and heteroaromatic groups may additionally be mono-, di- or trisubstituted in the carbon skeleton by fluorine, chlorine or bromine atoms, by alkyl groups, alkoxy, trifluoromethyl, nitro, hydroxy, amino, aminocarbonyl, acetyl or cyano groups and the substituents may be identical or different,

Y denotes the methylene or the —NH— group,

Y¹ denotes the carbon or the nitrogen atom,

X¹ denotes a pair of free electrons, if Y¹ denotes the nitrogen atom, or, if Y¹ is the carbon atom, the hydrogen atom or the carboxylic acid group optionally esterified with a lower aliphatic alcohol,

X³ and X⁴ each denote the hydrogen atom or the carboxylic acid group optionally esterified with a lower aliphatic alcohol,

with the proviso that at least one but also not more than one of the groups X¹, X², X³ or X⁴ contains an optionally esterified carboxylic acid function, and

R¹ denotes a group of general formula

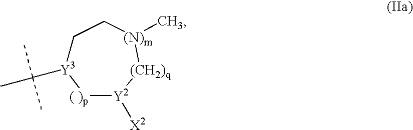

wherein

Y² denotes the carbon atom or, if m assumes the value 0, may also denote the nitrogen atom,

Y³, which is always different from Y¹, denotes the carbon or the nitrogen atom,

X² denotes a group of general formula

CH₂CO₂R², (III)

wherein

R² denotes the hydrogen atom or a C₁₋₅-alkyl group,

or, if Y² is the carbon atom, also denotes the hydrogen atom or the carboxylic acid group optionally esterified with a lower aliphatic alcohol,

m denotes the numbers 0 or 1,

p denotes the numbers 0, 1 or 2 and

q denotes the numbers 0, 1 or 2,

while the sum of m, p and q may assume the values 1 or 2,

or one of the groups

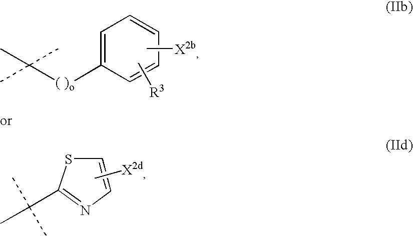

wherein

X^{2b} and X^{2d} each denote the hydrogen atom or the carboxylic acid group optionally esterified with a lower aliphatic alcohol,

o denotes the numbers 0, 1, 2 or 3 and

R³ denotes the hydrogen atom, the fluorine, chlorine or bromine atom, a methyl, methoxy, nitro, trifluoromethyl or cyano group,

while, unless otherwise stated, the above-mentioned alkyl groups or the alkyl groups contained in the above-mentioned groups contain 1 to 4 carbon atoms and may be branched or unbranched,

the tautomers, the diastereomers, the enantiomers and the salts thereof.

Particularly preferred compounds of the above general formula I are those wherein

R denotes a monounsaturated 5- to 7-membered diaza, triaza or S,S-dioxido-thiadiaza heterocycle,

while the above-mentioned heterocycles are linked via a nitrogen atom and

are characterised by a carbonyl group or sulphonyl group each flanked by two nitrogen atoms,

may be substituted at a carbon atom by a phenyl group,

and the double bond of one of the above-mentioned unsaturated heterocycles may be fused to a benzene, pyridine or quinoline ring,

while the phenyl groups contained in R as well as benzo-, pyrido- and quinolino-fused heterocycles may additionally be mono- or disubstituted in the carbon skeleton by fluorine, chlorine or bromine atoms, by methyl, methoxy, trifluoromethyl, or cyano groups, while the substituents may be identical or different,

Ar denotes a phenyl, 1-naphthyl, 2-naphthyl, 1,2,3,4-tetrahydro-1-naphthyl or 2,3-dihydrobenzo[b]fur-5-yl group,

while the above-mentioned aromatic and heteroaromatic groups may additionally be mono-, di- or trisubstituted in the carbon skeleton by fluorine, chlorine or bromine atoms, by methyl, methoxy, trifluoromethyl, hydroxy or amino groups and the substituents may be identical or different,

Y denotes the methylene or —NH— group,

Y¹ denotes the carbon or nitrogen atom,

X¹ denotes a pair of free electrons, if Y¹ denotes the nitrogen atom, or, if Y¹ is the carbon atom, the hydrogen atom or the carboxylic acid group optionally esterified with methanol or ethanol,

X³ and X⁴ each denote the hydrogen atom or the carboxylic acid group optionally esterified with methanol or ethanol,

with the proviso that at least one but also not more than one of the groups X¹, X², X³ or X⁴ contains an optionally esterified carboxylic acid function, and

R¹ denotes a group of general formula

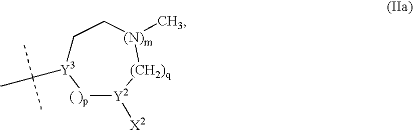

wherein

Y² denotes the carbon or, if m assumes the value 0, also denotes the nitrogen atom,

Y³, which is always different from Y¹, denotes the carbon or the nitrogen atom,

X² denotes a group of general formula

CH₂CO₂R², (III)

wherein

R² denotes the hydrogen atom or a straight-chain or branched C₁₋₄-alkyl group,

or, if Y² is the carbon atom, also denotes the hydrogen atom or the carboxylic acid group optionally esterified with methanol or ethanol,

m denotes the numbers 0 or 1,

p denotes the numbers 0, 1 or 2 and

q denotes the numbers 0, 1 or 2,

while the sum of m, p and q may assume the values 1 or 2,

or one of the groups

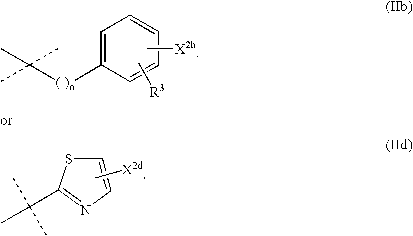

wherein

X^{2b} and X^{2d} each denote the hydrogen atom or the carboxylic acid group optionally esterified with methanol or ethanol,

o denotes the numbers 0, 1 or 2 and

R³ denotes the hydrogen atom, the fluorine, chlorine or bromine atom, a methyl, methoxy or trifluoromethyl group,

while, unless otherwise stated, the above-mentioned alkyl groups or the alkyl groups contained in the above-mentioned groups contain 1 to 4 carbon atoms and may be straight-chain or branched,

the tautomers, the diastereomers, the enantiomers and the salts thereof.

Most particularly preferred compounds of the above general formula (I) are those wherein

R denotes the 3,4-dihydro-2(1H)-oxoquinazolin-3-yl, 2,4-dihydro-5-phenyl-3(3H)-oxo-1,2,4-triazol-2-yl, 1,3-dihydro-2(2H)-oxoimidazo[4,5-c]quinolin-3-yl, 2-oxo-1,3,4,5-tetrahydro-1,3-benzodiazepin-3-yl, 3,4-dihydro-2(1H)-oxopyrido[3,4-d]pyrimidin-3-yl or 3,4-dihydro-2,2-dioxido-2, 1, 3-benzothiadiazin-3-yl group,

Ar denotes the 3,5-dibromo-4-hydroxyphenyl, 4-amino-3,5-dibromophenyl, 4-bromo-3,5-dimethylphenyl, 3,5-dichloro-4-methylphenyl, 3,4-dibromophenyl, 3-bromo-4,5-dimethylphenyl, 3,5-dibromo-4-methylphenyl, 3-chloro-4-methylphenyl, 3,4-difluorophenyl, 4-hydroxyphenyl, 1-naphthyl, 3,5-dibromo-4-fluorophenyl, 3,5-bis-(trifluoromethyl)-phenyl, 3,4,5-trimethylphenyl, 3-(trifluoromethyl)-phenyl, 3,5-dimethyl-4-methoxyphenyl, 4-amino-3,5-dichlorophenyl, 2,4-bis-(trifluoromethyl)-phenyl, 3,4,5-tribromophenyl, 3,4-dimethoxyphenyl, 3,4-dichlorophenyl, 4-bromo-3,5-dichlorophenyl, 2-naphthyl, 2,3-dihydrobenzo[b]fur-5-yl, 1,2,3,4-tetrahydro-1-naphthyl or 2,3-dichlorophenyl group,

Y denotes the methylene or the —NH— group,

Y¹ denotes the carbon or the nitrogen atom,

X¹ denotes a pair of free electrons, if Y¹ denotes the nitrogen atom, or, if Y¹ is the carbon atom, the hydrogen atom, the carboxylic acid or the methoxycarbonyl group and

R¹ denotes a group of general formula

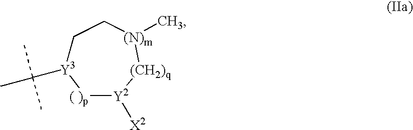

wherein

Y² denotes the carbon atom or, if m assumes the value 0, also the nitrogen atom,

Y³, which is always different from Y¹, denotes the carbon or the nitrogen atom,

X² denotes a group of general formula

CH₂CO₂R², (III)

wherein

R² denotes the hydrogen atom or a straight-chain or branched C₁₋₄-alkyl group,

or, if Y² is the carbon atom, also denotes the hydrogen atom or the carboxylic acid group optionally esterified with methanol or ethanol,

m denotes the numbers 0 or 1,

p and q in each case denotes the numbers 0, 1 or 2,

while the sum of m, p and q may assume the values 1 or 2,

or one of the groups

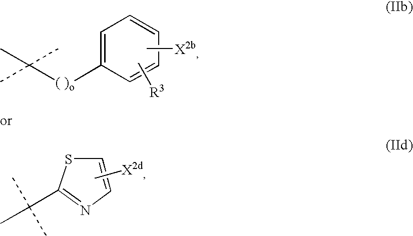

wherein

X^{2b} denotes the hydrogen atom or the carboxylic acid group optionally esterified with methanol or ethanol,

X^{2d} denotes the hydrogen atom or the carboxylic acid group optionally esterified with methanol,

o denotes the numbers 0, 1 or 2 and

R³ denotes the hydrogen atom or the trifluoromethyl group,

while, unless otherwise stated, the above-mentioned alkyl groups or the alkyl groups contained in the above-mentioned groups contain 1 to 4 carbon atoms and may be straight-chain or branched,

the tautomers, the diastereomers, the enantiomers and the salts thereof.

The following are mentioned as examples of particularly preferred compounds:

(1) ethyl 4-{1-[3,5-dibromo-N-[[4-(3,4-dihydro-2(1H)-oxoquinazolin-3-yl)-1-piperidinyl]carbonyl]-D-tyrosyl]-4-piperidinyl}-1-piperazineacetate,

(2) 4-{1-[3,5-dibromo-N-[[4-(3,4-dihydro-2(1H)-oxoquinazolin-3-yl)-1-piperidinyl]carbonyl]-D-tyrosyl]-4-piperidinyl}-1-piperazineacetic acid,

(3) 1,1-dimethylethyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-1-piperidineacetate,

(4) 4-{4-[3,5-dibromo-N-[[4-(3,4-dihydro-2(1H)-oxoquinazolin-3-yl)-1-piperidinyl]carbonyl]-D-tyrosyl]-1-piperazinyl}-1-piperidineacetic acid,

(5) methyl 1′-[3,5-dibromo-N-[[4-(3,4-dihydro-2(1H)-oxoquinazolin-3-yl)-1-piperidinyl]carbonyl]-D-tyrosyl]-[1,4′]bipiperidinyl-4-acetate,

(6) 1′-[3,5-dibromo-N-[[4-(3,4-dihydro-2(1H)-oxoquinazolin-3-yl)-1-piperidinyl]-carbonyl]-D-tyrosyl]-[1,4′]bipiperidinyl-4-acetic acid,

(7) ethyl endo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylate,

(8) endo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylic acid,

(9) ethyl exo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylate,

(10) exo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylic acid,

(11) ethyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-1-piperidineacetate,

(12) methyl 1′-[4-amino-3,5-dibromo-N-[[4-(2-oxo-1,3,4,5-tetrahydro-1,3-benzodiazepin-3-yl)-1-piperidinyl]carbonyl]-D-phenylalanyl]-[1,4′]bipiperidinyl-4-acetate,

(13) 1′-[4-amino-3,5-dibromo-N-[[4-(2-oxo-1,3,4,5-tetrahydro-1,3-benzodiazepin-3-yl)-1-piperidinyl]carbonyl]-D-phenylalanyl]-[1,4′]bipiperidinyl-4-acetic acid,

(14) ethyl 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-1-piperidineacetate,

(15) ethyl 4-{1-[4-bromo-N-[[4-(3,4-dihydro-2(1H)-oxoquinazolin-3-yl)-1-piperidinyl]carbonyl]-3,5-dimethyl-D,L-phenylalanyl]-4-piperidinyl}-1-piperazineacetate,

(16) ethyl 4-{1-[3,5-dichloro-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetate,

(17) ethyl 4-{1-[3,4-dibromo-N-[[4-(3,4-dihydro-2(1H)-oxoquinazolin-3-yl)-1-piperidinyl]carbonyl]-D,L-phenylalanyl]-4-piperidinyl}-1-piperazineacetate,

(18) ethyl 4-{1-[3-bromo-N-[[4-(3,4-dihydro-2(1H)-oxoquinazolin-3-yl)-1-piperidinyl]carbonyl]-4,5-dimethyl-D,L-phenylalanyl]-4-piperidinyl}-1-piperazineacetate,

(19) ethyl 4-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetate,

(20) ethyl 4-{1-[3-chloro-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetate,

(21) ethyl 4-{4-[4-bromo-N-[[4-(3,4-dihydro-2(1H)-oxoquinazolin-3-yl)-1-piperidinyl]carbonyl]-3,5-dimethyl-D,L-phenylalanyl]-1-piperazinyl}-1-piperidineacetate,

(22) 4-{1-[4-bromo-N-[[4-(3,4-dihydro-2(1H)-oxoquinazolin-3-yl)-1-piperidinyl]-carbonyl]-3,5-dimethyl-D,L-phenylalanyl]-4-piperidinyl}-1-piperazineacetic acid,

(23) 4-{1-[3,5-dichloro-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetic acid,

(24) 4-{1-[3,4-dibromo-N-[[4-(3,4-dihydro-2(1H)-oxoquinazolin-3-yl)-1-piperidinyl]carbonyl]-D,L-phenylalanyl]-4-piperidinyl}-1-piperazineacetic acid,

(25) 4-{1-[3-bromo-N-[[4-(3,4-dihydro-2(1H)-oxoquinazolin-3-yl)-1-piperidinyl]-carbonyl]-4,5-dimethyl-D,L-phenylalanyl]-4-piperidinyl}-1-piperazineacetic acid,

(26) 4-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}1-piperazineacetic acid,

(27) 4-{1-[3-chloro-N-[[4-(3,4-dihydro-2(1H)-oxoquinazolin-3-yl)-1-piperidinyl]-carbonyl]-4-methyl-D,L-phenylalanyl]-4-piperidinyl}-1-piperazineacetic acid,

(28) 4-{4-[4-bromo-N-[[4-(3,4-dihydro-2(1H)-oxoquinazolin-3-yl)-1-piperidinyl]-carbonyl]-3,5-dimethyl-D,L-phenylalanyl]-1-piperazinyl}-1-piperidineacetic acid,

(29) 1,1-dimethylethyl 4-{1-[3,4-difluoro-N-[[4-(3,4-dihydro-2(1H)-oxoquinazolin-3-yl)-1-piperidinyl]carbonyl]-D,L-phenylalanyl]-4-piperidinyl}-1-piperazineacetate,

(30) methyl 1′-[N-[[4-(3,4-dihydro-2(1H)-oxoquinazolin-3-yl)-1-piperidinyl]carbonyl]-D-tyrosyl]-[1,4′]bipiperidinyl-4-acetate,

(31) ethyl 4-{1-[N-[[4-(3,4-dihydro-2(1H)-oxoquinazolin-3-yl)-1-piperidinyl]carbonyl]-D-tyrosyl]-4-piperidinyl}-1-piperazineacetate,

(32) ethyl(R,S)-4-{1-[2-[(3,5-dibromo-4-methylphenyl)methyl]-4-[4-(3,4-dihydro-2(1H)-oxoquinazolin-3-yl)-1-piperidinyl]-1,4-dioxobutyl]-4-piperidinyl}-1-piperazineacetate,

(33) methyl 1-{1-[4-amino-3,5-dibromo-N-[[4-(3,4-dihydro-2(H)-oxoquinazolin-3-yl)-1-piperidinyl]carbonyl]-D-phenylalanyl]-4-piperidinyl}-(S)-pyrrolidine-2-carboxylate,

(34) methyl 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(S)-pyrrolidine-2-carboxylate,

(35) 1-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-(S)-pyrrolidine-2-carboxylic acid,

(36) 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(S)-pyrrolidine-2-carboxylic acid,

(37) methyl 1-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-(R)-pyrrolidine-2-carboxylate,

(38) methyl 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(R)-pyrrolidine-2-carboxylate,

(39) 1-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-(R)-pyrrolidine-2-carboxylic acid,

(40) 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(R)-pyrrolidine-2-carboxylic acid,

(41) methyl 1′-[4-amino-3,5-dibromo-N-[[4-(3,4-dihydro-2(1H)-oxoquinazolin-3-yl)-1-piperidinyl]carbonyl]-D-phenylalanyl]-(R)-[1,4′]bipiperidinyl-2-carboxylate,

(42) methyl 1′-[3,5-dibromo-N-[[4-(3,4-dihydro-2(1H)-oxoquinazolin-3-yl)-1-piperidinyl]carbonyl]-D-tyrosyl]-(R)-[1,4′]bipiperidinyl-2-carboxylate,

(43) methyl 1′-[4-amino-3,5-dibromo-N-[[4-(3,4-dihydro-2(1H)-oxoquinazolin-3-yl)-1-piperidinyl]carbonyl]-D-phenylalanyl]-(S)-[1,4′]bipiperidinyl-2-carboxylate,

(44) methyl 1′-[3,5-dibromo-N-[[4-(3,4-dihydro-2(1H)-oxoquinazolin-3-yl)-1-piperidinyl]carbonyl]-D-tyrosyl]-(S)-[1,4′]bipiperidinyl-2-carboxylate,

(45) 1′-[4-amino-3,5-dibromo-N-[[4-(3,4-dihydro-2(1H)-oxoquinazolin-3-yl)-1-piperidinyl]carbonyl]-D-phenylalanyl]-(R)-[1,4′]bipiperidinyl-2-carboxylic acid,

(46) 1′-[3,5-dibromo-N-[[4-(3,4-dihydro-2(1H)-oxoquinazolin-3-yl)-1-piperidinyl]-carbonyl]-D-tyrosyl]-(R)-[1,4′]bipiperidinyl-2-carboxylic acid,

(47) 1′-[3,5-dibromo-N-[[4-(3,4-dihydro-2(1H)-oxoquinazolin-3-yl)-1-piperidinyl]-carbonyl]-D-tyrosyl]-(S)-[1,4′]bipiperidinyl-2-carboxylic acid,

(48) 1′-[4-amino-3,5-dibromo-N-[[4-(3,4-dihydro-2(1H)-oxoquinazolin-3-yl)-1-piperidinyl]carbonyl]-D-phenylalanyl]-(S)-[1,4′]bipiperidinyl-2-carboxylic acid,

(49) methyl 1′-[3,5-dibromo-N-[[4-(3,4-dihydro-2(1H)-oxoquinazolin-3-yl)-1-piperidinyl]carbonyl]-D-tyrosyl]-[1,4′]bipiperidinyl-4′-carboxylate,

(50) methyl 1′-[4-amino-3,5-dibromo-N-[[4-(3,4-dihydro-2(1H)-oxoquinazolin-3-yl)-1-piperidinyl]carbonyl]-D-phenylalanyl]-[1,4′]bipiperidinyl-4′-carboxylate,

(51) 1′-[3,5-dibromo-N-[[4-(3,4-dihydro-2(1H)-oxoquinazolin-3-yl)-1-piperidinyl]-carbonyl]-D-tyrosyl]-[1,4′]bipiperidinyl-4′-carboxylic acid,

(52) 1′-[4-amino-3,5-dibromo-N-[[4-(3,4-dihydro-2(1H)-oxoquinazolin-3-yl)-1-piperidinyl]carbonyl]-D-phenylalanyl]-[1,4′]bipiperidinyl-4′-carboxylic acid,

(53) 1′-[N-[[4-(3,4-dihydro-2(1H)-oxoquinazolin-3-yl)-1-piperidinyl]carbonyl]-D-tyrosyl]-[1,4′]bipiperidinyl-4-acetic acid,

(54) 4-{1-[N-[[4-(3,4-dihydro-2(1H)-oxoquinazolin-3-yl)-1-piperidinyl]carbonyl]-D-tyrosyl]-4-piperidinyl}-1-piperazineacetic acid,

(55) ethyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoate,

(56) ethyl 3-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoate,

(57) methyl 4-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoate,

(58) ethyl 4-{1-[3,5-dibromo-N-[[4-(3,4-dihydro-2(1H)-oxoquinazolin-3-yl)-1-piperidinyl]carbonyl]-D-tyrosyl]-4-piperidinylmethyl}-benzoate,

(59) ethyl 4-{2-[1-[3,5-dibromo-N-[[4-(3,4-dihydro-2(1H)-oxoquinazolin-3-yl)-1-piperidinyl]carbonyl]-D-tyrosyl]-4-piperidinyl]-ethyl}-benzoate,

(60) methyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-3-(trifluoromethyl)-benzoate,

(61) methyl 3-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoate,

(62) ethyl 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoate,

(63) ethyl 3-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoate,

(64) methyl 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-benzoate,

(65) methyl 4-{2-[1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl]-ethyl}-benzoate,

(66) methyl 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-3-(trifluoromethyl)-benzoate,

(67) methyl 3-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-benzoate,

(68) 4-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoic acid,

(69) 3-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoic acid,

(70) 4-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoic acid,

(71) 4-{1-[3,5-dibromo-N-[[4-(3,4-dihydro-2(1H)-oxoquinazolin-3-yl)-1-piperidinyl]carbonyl]-D-tyrosyl]-4-piperidinylmethyl}-benzoic acid,

(72) 4-{2-[1-[3,5-dibromo-N-[[4-(3,4-dihydro-2(1H)-oxoquinazolin-3-yl)-1-piperidinyl]carbonyl]-D-tyrosyl]-4-piperidinyl]-ethyl}-benzoic acid,

(73) 4-{4-[3,5-dibromo-N-[[4-(3,4-dihydro-2(1H)-oxoquinazolin-3-yl)-1-piperidinyl]carbonyl]-D-tyrosyl]-1-piperazinyl}-3-(trifluoromethyl)-benzoic acid,

(74) 3-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoic acid,

(75) 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoic acid,

(76) 3-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoic acid,

(77) 4-{1-[4-amino-3,5-dibromo-N-[[4-(2-oxo-1,3,4,5-terahydro-1,3-benzodiazepin-3-yl)-1-piperidinyl]carbonyl]-D-phenylalanyl]-4-piperidinyl}-benzoic acid,

(78) 4-{2-[1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl]-ethyl}-benzoic acid,

(79) 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-3-(trifluoromethyl)-benzoic acid,

(80) 3-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-benzoic acid,

(81) ethyl 4-{1-[3-(1-naphthyl)-N-[[4-(2-oxo-1,3,4,5-tetrahydro-1,3-benzodiazepin-3-yl)-1-piperidinyl]carbonyl]-D-alanyl]-4-piperidinyl}-1-piperazineacetate,

(82) 4-{1-[3-(1-naphthyl)-N-[[4-(2-oxo-1,3,4,5-tetrahydro-1,3-benzodiazepin -3-yl)-1-poperidinyl]carbonyl]-D-alanyl]-4-piperidinyl}-1-piperazineacetic acid,

(83) methyl 2-{4-[3,5-dibromo-N-[[4-(3,4-dihydro-2(1H)-oxoquinazolin-3-yl)-1-piperidinyl]carbonyl]-D-tyrosyl]-1-piperazinyl}-5-thiazolecarboxylate,

(84) methyl 2-{4-[3,5-dibromo-N-[[4-(3,4-dihydro-2(1H)-oxoquinazolin-3-yl)-1-piperidinyl]carbonyl]-D-tyrosyl]-1-piperazinyl}-4-thiazolecarboxylate,

(85) 2-{4-[3,5-dibromo-N-[[4-(3,4-dihydro-2(1H)-oxoquinazolin-3-yl)-1-piperidinyl]carbonyl]-D-tyrosyl]-1-piperazinyl}-5-thiazolecarboxylic acid,

(86) 2-{4-[3,5-dibromo-N-[[4-(3,4-dihydro-2(1H)-oxoquinazolin-3-yl)-1-piperidinyl]carbonyl]-D-tyrosyl]-1-piperazinyl}-4-thiazolecarboxylic acid,

(87) methyl 2-{4-[4-amino-3,5-dibromo-N-[[4-(3,4-dihydro-2(1H)-oxoquinazolin-3-yl)-1-piperidinyl]carbonyl]-D-phenylalanyl]-1-piperazinyl}-4-thiazolecarboxylate,

(88) methyl 2-{4-[4-amino-3,5-dibromo-N-[[4-(3,4-dihydro-2(1H)-oxoquinazolin-3-yl)-1-piperidinyl]carbonyl]-D-phenylalanyl]-1-piperazinyl}-5-thiazolecarboxylate,

(89) 2-{4-[4-amino-3,5-dibromo-N-[[4-(3,4-dihydro-2(1H)-oxoquinazolin -3-yl)-1-piperidinyl]carbonyl]-D-phenylalanyl]-1-piperazinyl}-4-thiazolecarboxylic acid,

(90) 2-{4-[4-amino-3,5-dibromo-N-[[4-(3,4-dihydro-2(1H)-oxoquinazolin -3-yl)-1-piperidinyl]carbonyl]-D-phenylalanyl]-1-piperazinyl}-5-thiazolecarboxylic acid,

(91) 4-{4-[4-amino-3,5-dibromo-N-[[4-(3,4-dihydro-2(1H)-oxoquinazolin -3-yl)-1-piperidinyl]carbonyl]-D-phenylalanyl]-1-piperazinyl}-1-piperidineacetic acid,

(92) 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-1-piperidineacetic acid,

(93) 1,1-dimethylethyl 4-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-1-piperazineacetate,

(94) 1,1-dimethylethyl 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-1-piperazineacetate,

(95) ethyl 4-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-1-piperazineacetate,

(96) ethyl 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-1-piperazineacetate,

(97) 4-{1-[4-amino-3,5-dibromo-N-[[4-(3,4-dihydro-2(1H)-oxoquinazolin- 3-yl)-1-piperidinyl]carbonyl]-D-phenylalanyl]-4-piperidinyl}-1-piperazineacetic acid,

(98) 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-1-piperazineacetic acid,

(99) (R,S)-4-{1-[2-[(4-amino-3,5-dibromophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,

(100) (R,S)-4-{1-[2-[(3,5-dibromo-4-fluorophenyl)methyl]-4-[4-(3,4-dihydro -2(1H)-oxoquinazolin-3-yl)-1-piperidinyl]-1,4-dioxobutyl]-4-piperidinyl}-1-piperazineacetic acid,

(101) (R,S)-4-{1-[4-[4-(3,4-dihydro-2(1H)-oxopyrido[3,4-d]pyrimidin-3-yl)-1-piperidinyl]-2-[(1-naphthyl)methyl]-1,4-dioxobutyl]-4-piperidinyl}-1-piperazineacetic acid,

(102) (R,S)-4-{1-[2-[[3,5-bis-(trifluoromethyl)-phenyl]methyl]-4-[4-(3,4-dihydro-2(1H)-oxoquinazolin-3-yl)-1-piperidinyl]-1,4-dioxobutyl]-4-piperidinyl}-1-piperazineacetic acid,

(103) (R,S)₄-{1-[4-[4-(3,4-dihydro-2(1H)-oxoquinazolin-3-yl)-1-piperidinyl]-2-[(3,4,5-trimethylphenyl)methyl]-1,4-dioxobutyl]-4-piperidinyl}-1-piperazineacetic acid,

(104) (R,S)-4-{1-[2-[(3-bromo-4,5-dimethylphenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,

(105) (R,S)-4-{1-[4-[4-(3,4-dihydro-2(1H)-oxoquinazolin-3-yl)-1-piperidinyl]-2-[[3-(trifluoromethyl)-phenyl]methyl]-1,4-dioxobutyl]-4-piperidinyl}-1-piperazineacetic acid,

(106) (R,S)-4-{1-[4-[4-(3,4-dihydro-2(1H)-oxoquinazolin-3-yl)-1-piperidinyl]-2-[(4-methoxy-3,5-dimethylphenyl)methyl]-1,4-dioxobutyl]-4-piperidinyl}-1-piperazineacetic acid,

(107) (R,S)-4-{1-[2-[(4-amino-3,5-dichlorophenyl)methyl]-4-[4-(3,4-dihydro -2(1H)-oxoquinazolin-3-yl)-1-piperidinyl]-1,4-dioxobutyl]-4-piperidinyl}-1-piperazineacetic acid,

(108) (R,S)-4-{1-[2-[[2,4-bis-(trifluoromethyl)-phenyl]methyl]-4-[4-(3,4-dihydro-2(1H)-oxoquinazolin-3-yl)-1-piperidinyl]-1,4-dioxobutyl]-4-piperidinyl}-1-piperazineacetic acid,

(109) (R,S)-4-{1-[2-[(3,5-dibromo-4-methylphenyl)methyl]-4-[4-(3,4-dihydro -2(1H)-3-yl)-1-piperidinyl]-1,4-dioxobutyl]-4-piperidinyl}-1-piperazineacetic acid,

(110) (R,S)-4-{1-[4-[4-(3,4-dihydro-2(1H)-oxoquinazolin-3-yl)-1-piperidinyl]-2-[(3,4,5-tribromophenyl)methyl]-1,4-dioxobutyl]-4-piperidinyl)-1-piperazineacetic acid,

(111) (R,S)-4-{1-[4-[4-(3,4-dihydro-2(1H)-oxoquinazolin-3-yl)-1-piperidinyl]-2-[(3,4-dimethoxyphenyl)methyl]-1,4-dioxobutyl]-4-piperidinyl}-1-piperazineacetic acid,

(112) (R,S)-4-(1-[2-[(3,4-dichlorophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,

(113) (R,S)-4-{1-[2-[(4-bromo-3,5-dichlorophenyl)methyl]-4-[4-(3,4-dihydro -2(1H)-oxoquinazolin-3-yl)-1-piperidinyl]-1,4-dioxobutyl]-4-piperidinyl}-1-piperazineacetic acid,

(114) (R,S)-4-{1-[4-[4-(3,4-dihydro-2(1H)-oxoquinazolin-3-yl)-1-piperidinyl]-2-[(2-naphthyl)methyl]-1,4-dioxobutyl]-4-piperidinyl}-1-piperazineacetic acid,

(115) (R,S)-4-{1-[2-[(2,3-dihydrobenzo[b]fur-5-yl)methyl]-4-[4-(3,4-dihydro-2(1H)-oxoquinazolin-3-yl)-1-piperidinyl]-1,4-dioxobutyl]-4-piperidinyl}-1-piperazineacetic acid,

(116) (R,S)-4-{1-[4-[4-(3,4-dihydro-2(1H)-oxoquinazolin-3-yl)-1-piperidinyl]-2-[(1,2,3,4-tetrahydro-1-naphthyl)methyl]-1,4-dioxobutyl]-4-piperidinyl}-1-piperazineacetic acid,

(117) (R,S)-4-{1-[2-[(3,5-dibromo-4-methylphenyl)methyl]-4-[4-(3,4-dihydro-2,2-dioxido-2,1,3-benzothiadiazin-3-yl)-1-piperidinyl]-1,4-dioxobutyl]-4-piperidinyl}-1-piperazineacetic acid,

(118) (R,S)-4-{1-[2-[(2,3-dichlorophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,

(119) ethyl(R,S)-4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-piperazineacetate,

(120) (R,S)-4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,

(121) (R,S)-4-{4-[2-[(3,4-dibromophenyl)methyl]-4-[4-(3,4-dihydro-2(1H)-oxoquinazolin-3-yl)-1-piperidinyl]-1,4-dioxobutyl]-1-piperazinyl}-1-piperidineacetic acid,

(122) methyl 1-{1-[3,5-dibromo-N-[[4-(3,4-dihydro-2(1H)-oxoquinazolin-3-yl) -1-piperidinyl]carbonyl]-4-methyl-D,L-phenylalanyl]-4-piperidinyl}-(S)-pyrrolidine-2-carboxylate,

(123) methyl 1-{1-[3-chloro-N-[[4-(3,4-dihydro-2(1H)-oxoquinazolin-3-yl)-1-piperidinyl]carbonyl]-4-methyl-D,L-phenylalanyl]-4-piperidinyl}-(S)-pyrrolidine-2-carboxylate,

(124) methyl 1-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-(R)-pyrrolidine-2-carboxylate,

(125) methyl 1-{1-[3-chloro-N-[[4-(3,4-dihydro-2(1H)-oxoquinazolin-3-yl)-1-piperidinyl]carbonyl]-4-methyl-D,L-phenylalanyl]-4-piperidinyl}-(R)-pyrrolidine-2-carboxylate,

(126) 1-{1-[3,5-dibromo-N-[[4-(3,4-dihydro-2(1H)-oxoquinazolin-3-yl) 1-piperidinyl]carbonyl]-4-methyl-D,L-phenylalanyl]-4-piperidinyl}-(S)-pyrrolidine-2-carboxylic acid,

(127) 1-{1-[3-chloro-N-[[4-(3,4-dihydro-2(1H)-oxoquinazolin-3-yl)-1-piperidinyl]-carbonyl]-4-methyl-D,L-phenylalanyl]-4-piperidinyl}-(S)-pyrrolidine-2-carboxylic acid,

(128) ethyl 4-{1-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-4-piperidinyl}-1-methyl-2-piperazinecarboxylate,

(129) ethyl 4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylate,

(130) ethyl 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-2-piperazinecarboxylate,

(131) ethyl 4-{1-[2-[(3,5-dibromo-4-methylphenyl)methyl]-4-[4-(3,4-dihydro 3-yl)-1-piperidinyl]-1,4-dioxobutyl]-4-piperidinyl}-1-methyl-2-piperazinecarboxylate,

(132) ethyl 4-[2-[(3,5-dibromo-4-methylphenyl)methyl]-4-[4-(3,4-dihydro-2(1H)-oxoquinazolin-3-yl)-1-piperidinyl]-1,4-dioxobutyl]-1-(1-methyl-4-piperidinyl)-2-piperazinecarboxylate,

(133) ethyl 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylate,

(134) 4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylic acid,

(135) 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-2-piperazinecarboxylic acid,

(136) 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylic acid,

(137) 4-{1-[2-[(3,5-dibromo-4-methylphenyl)methyl]-4-[4-(3,4-dihydro-2(H)-oxo-quinazolin-3-yl)-1-piperidinyl]-1,4-dioxobutyl]-4-piperidinyl}-1-methyl-2-piperazinecarboxylic acid,

(138) ethyl 4-[2-[(3,5-dibromo-4-methylphenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylate,

(139) 4-[2-[(3,5-dibromo-4-methylphenyl)methyl]-4-[4-(3,4-dihydro-2(1H)-oxo-quinazolin-3-yl)-1-piperidinyl]-1,4-dioxobutyl]-1-(1-methyl-4-piperidinyl)-2-piperazinecarboxylic acid,

(140) ethyl 4-[2-[(4-amino-3,5-dibromophenyl)methyl]-4-[4-(3,4-dihydro-2(1H)-oxoquinazolin-3-yl)-1-piperidinyl]-1,4-dioxobutyl)-1-(1-methyl-4-piperidinyl)-2-piperazinecarboxylate,

(141) ethyl 4-[2-[(4-amino-3,5-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylate,

(142) ethyl 4-{1-[2-[(4-amino-3,5-dibromophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylate,

(143) 4-[2-[(4-amino-3,5-dibromophenyl)methyl]-4-[4-(3,4-dihydro-2(1H)-oxo-quinazolin-3-yl)-1-piperidinyl]-1,4-dioxobutyl]-1-(1-methyl-4-piperidinyl)-2-piperazinecarboxylic acid,

(144) 4-{1-[2-[(4-amino-3,5-dibromophenyl)methyl]-4-[4-(3,4-dihydro-2(1H)-oxo-quinazolin-3-yl)-1-piperidinyl]-1,4-dioxobutyl]-4-piperidinyl}-1-methyl-2-piperazinecarboxylic acid,

(145) 4-[2-[(4-amino-3,5-dibromophenyl)methyl]-4-[4-(3,4-dihydro-2(1H)-oxo-quinazolin-3-yl)-1-piperidinyl]-1,4-dioxobutyl]-1-(1-methyl-4-piperidinyl)-3-piperazinecarboxylic acid,

(146) 4-[2-[(3,5-dibromo-4-methylphenyl)methyl]-4-[4-(3,4-dihydro-2(1H)-oxo-quinazolin-3-yl)-1-piperidinyl]-1,4-dioxobutyl]-1-(1-methyl-4-piperidinyl)-3-piperazinecarboxylic acid,

(147) ethyl 4-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-1-(1-methyl-4-piperidinyl)-2-piperazinecarboxylate,

(148) ethyl 4-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-1-(1-methyl-4-piperidinyl)-3-piperazinecarboxylate,

(149) 4-{1-[4-[4-(3,4-dihydro-2(1H)-oxoquinazolin-3-yl)-1-piperidinyl]-2-[(3,5-di-methyl-4-hydroxyphenyl)methyl]-1,4-dioxobutyl]-4-piperidinyl}-1-methyl-2-piperazinecarboxylic acid,

(150) ethyl 4-{1-[2-[(4-bromo-3,5-dichlorophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylate,

(151) ethyl 1-{1-[2-[(3,4-dibromophenyl)methyl]4-[4-(3,4-dihydro-2(1H)-oxoquinazolin-3-yl)-1-piperidinyl]-1,4-dioxobutyl]-4-piperidinyl}-4-methyl-2-piperazinecarboxylate,

(152) ethyl 1-{1-[2-[(3,5-dibromo-4-methylphenyl)methyl]4-[4-(3,4-dihydro-2(1H)-oxoquinazolin-3-yl)-1-piperidinyl]-1,4-dioxobutyl]-4-piperidinyl}-4-methyl-2-piperazinecarboxylate,

(153) ethyl 1-{1-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-4-piperidinyl}-4-methyl-2-piperazinecarboxylate,

(154) ethyl 1-{1-[2-[(4-amino-3,5-dibromophenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylate,

(155) ethyl 1-{1-[2-[(4-bromo-3,5-dichlorophenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylate,

(156) 1-{1-[2-[(3,5-dibromo-4-methylphenyl)methyl]-4-[4-(3,4-dihydro-2(1H)-oxo-quinazolin-3-yl)-1-piperidinyl]-1,4-dioxobutyl]-4-piperidinyl}-4-methyl-2-piperazinecarboxylic acid,

(157) 1-{1-[4-[4-(3,4-dihydro-2(1H)-oxoquinazolin-3-yl)-1-piperidinyl]-2-[(3,5-di-methyl-4-hydroxyphenyl)methyl]-1,4-dioxobutyl]-4-piperidinyl}-4-methyl-2-piperazinecarboxylic acid,

(158) 1-{1-[2-[(4-amino-3,5-dibromophenyl)methyl]-4-[4-(3,4-dihydro-2(1H)-oxo-quinazolin-3-yl)-1-piperidinyl]-1,4-dioxobutyl]-4-piperidinyl}-4-methyl-2-piperazinecarboxylic acid,

(159) 1-{1-[2-[(4-bromo-3,5-dichlorophenyl)methyl]-4-[4-(3,4-dihydro-2(1H)-oxo-quinazolin-3-yl)-1-piperidinyl]-1,4-dioxobutyl]-4-piperidinyl}-4-methyl-2-piperazinecarboxylic acid,

(160) 1-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylic acid,

(161) ethyl 4-{1-[3,4-dibromo-N-[[4-(3,4-dihydro-2(1H)-oxoquinazolin-3-yl)-1-piperidinyl]carbonyl]-phenylalanyl]-4-piperidinyl}-1-methyl-2-piperazinecarboxylate,

(162) 4-{1-[2-[(4-bromo-3,5-dichlorophenyl)methyl]-4-[4-(3,4-dihydro-2(1H)-oxo-quinazolin-3-yl)-1-piperidinyl]-1,4-dioxobutyl]-4-piperidinyl}-1-methyl-2-piperazinecarboxylic acid,

(163) methyl 1′-[3,5-dibromo-N-[[4-(3,4-dihydro-2(1H)-oxoquinazolin-3-yl)-1-piperidinyl]carbonyl]-phenylalanyl]-[1,4′]bipiperidinyl-4-acetate,

(164) 1′-[3,5-dibromo-N-[[4-(3,4-dihydro-2(1H)-oxoquinazolin-3-yl)-1-piperidinyl]-carbonyl]-phenylalanyl]-[1,4′]bipiperidinyl-4-acetic acid,

(165) ethyl 4-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-1-methyl-2-piperazinecarboxylate,

(166) ethyl 1-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-4-methyl-2-piperazinecarboxylate and the salts thereof.

The compounds of general formula I are prepared by methods known in principle. The following methods have proved particularly suitable for preparing the compounds of general formula I according to the invention:

In order to prepare compounds of general formula (I) wherein Y denotes the NH group and neither X¹ nor X³ nor X⁴ nor R¹ contains a free carboxylic acid function, but otherwise all groups are as hereinbefore defined:

reacting piperidines of general formula

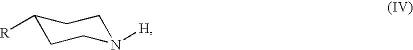

wherein

R is as hereinbefore defined, with carbonic acid derivatives of general formula

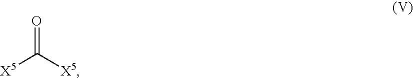

wherein

X⁵ denotes a nucleofugic group, preferably the 1H-imidazol-1-yl, 1H-1,2,4-triazol-1-yl, trichloromethoxy or the 2,5-dioxopyrrolidin-1-yloxy group,

and with primary amines of general formula

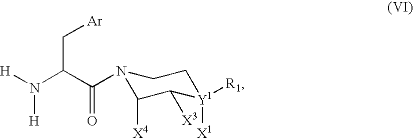

wherein

neither X¹ nor X³ nor X⁴ nor R¹ contains a free carboxylic acid function, but otherwise all groups are as hereinbefore defined.

The fundamentally two-step reactions are normally carried out as one-pot processes, in which, preferably, in the first step, one of the two components (IV) or (VI) is reacted with equimolar amounts of the carbonic acid derivative of general formula (V) in a suitable solvent at lower temperature, then at least equimolar amounts of the other component (IV) or (VI) are added and the reaction is completed at a higher temperature. The reactions with bis-(trichloromethyl)-carbonate are preferably carried out in the presence of at least 2 equivalents (based on bis-(trichloromethyl)-carbonate) of a tertiary base, for example triethylamine, N-ethyldiisopropylamine, pyridine, 1,5-diaza-bicyclo-[4,3,0]-non-5-ene, 1,4-diazabicyclo[2,2,2]octane or 1,8-diazabicyclo-[5,4,0]-undec-7-ene. The solvents used, which should be anhydrous, may be for example tetrahydrofuran, dioxane, dimethylformamide, dimethylacetamide, N-methyl-2-pyrrolidone, 1,3-dimethyl-2-imidazolidinone or acetonitrile, while if bis-(trichloromethyl)-carbonate is used as the carbonyl component anhydrous chlorohydrocarbons, for example dichloromethane, 1,2-dichloroethane or trichloroethylene are preferred. The reaction temperatures for the first reaction step are between −30° C. and +25° C., preferably-5° C. and +10° C., for the second reaction step between +15° C. and the boiling temperature of the solvent used, preferably between +20° C. and +70° C. (cf. also: H. A. Staab and W. Rohr, “Synthesen mit heterocyclischen Amiden (Azoliden)”, Neuere Methoden der Präparativen Organischen Chemie, Volume V, p. 53-93, Verlag Chemie, Weinheim/Bergstr., 1967; P. Majer and R. S. Randad, J. Org. Chem. 59, p.1937-1938 (1994); K. Takeda, Y. Akagi, A. Saiki, T. Sukahara and H. Ogura, Tetrahedron Letters 24 (42), 4569-4572 (1983)).

b) In order to prepare compounds of general formula (I) wherein Y denotes the CH₂ group and neither X¹ nor X³ nor X⁴ nor R¹ contains a free carboxylic acid function, but otherwise all groups are as hereinbefore defined:

Coupling a carboxylic acid of general formula

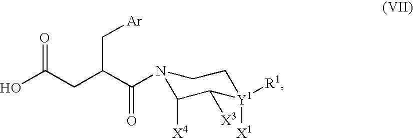

wherein

neither X¹ nor X³ nor X⁴ nor R¹ contains a free carboxylic acid function, but otherwise all groups are as hereinbefore defined,

with a piperidine of general formula

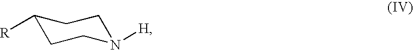

wherein

R has the meanings given hereinbefore.

The coupling is preferably carried out using methods known from peptide chemistry (cf. e.g. Houben-Weyl, Methoden der Organischen Chemie, Vol. 15/2), for example using carbodiimides such as e.g. dicyclohexylcarbodiimide (DCC), diisopropyl carbodiimide (DIC) or ethyl-(3-dimethylaminopropyl)-carbodiimide, O-(1H-benzotriazol-1-yl)-N,N-N′,N′-tetramethyluronium hexafluorophosphate (HBTU) or tetrafluoroborate (TBTU) or 1H-benzotriazol-1-yl-oxy-tris-(dimethylamino)-phosphonium hexafluorophosphate (BOP). By adding 1-hydroxybenzotriazole (HOBt) or 3-hydroxy-4-oxo-3,4-dihydro-1,2,3-benzotriazine (HOObt) the reaction speed can be increased. The couplings are normally carried out with equimolar amounts of the coupling components as well as the coupling reagent in solvents such as dichloromethane, tetrahydrofuran, acetonitrile, dimethyl formamide (DMF), dimethyl acetamide (DMA), N-methylpyrrolidone (NMP) or mixtures thereof and at temperatures between −30 and +30° C., preferably −20 and +25° C. If necessary, N-ethyl-diisopropylamine (DIEA) (Hünig base) is preferably used as an additional auxiliary base.

The so-called anhydride process is used as a further coupling method for synthesising compounds of general formula (I) (cf. also: M. Bodanszky, “Peptide Chemistry”, Springer-Verlag 1988, p. 58-59; M. Bodanszky, “Principles of Peptide Synthesis”, Springer-Verlag 1984, p. 21-27). The Vaughan variant of the mixed anhydride process is preferred (J. R. Vaughan Jr., J. Amer. Chem. Soc. 73, 3547 (1951)), in which the mixed anhydride of the carboxylic acid of general formula (VII) which is to be coupled and monoisobutyl carbonate is obtained, using isobutyl chlorocarbonate in the presence of bases such as 4-methyl-morpholine or 4-ethylmorpholine. The preparation of this mixed anhydride and the coupling with amines are carried out in a one-pot process, using the above-mentioned solvents and at temperatures between −20 and +25° C., preferably 0° C. and +25° C.

c) In order to prepare compounds of general formula (I) wherein Y denotes the CH₂ group and neither X¹ nor X³ nor X⁴ nor R¹ contains a free carboxylic acid function, but otherwise all groups are as hereinbefore defined:

Coupling a compound of general formula

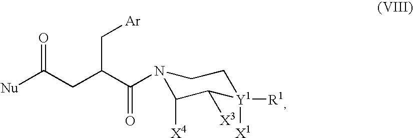

wherein

neither X¹ nor X³ nor X⁴ nor R¹ contains a free carboxylic acid function, but otherwise all groups are as hereinbefore defined, and Nu denotes a leaving group, for example a halogen atom, such as the chlorine, bromine or iodine atom, a C₁₋₁₀-alkylsulphonyloxy group, a phenylsulphonyloxy or naphthylsulphonyloxy group optionally mono-, di- or trisubstituted by chlorine or bromine atoms, by methyl or nitro groups, while the substituents may be identical or different, a 1H-imidazol-1-yl, a 1H-pyrazol-1-yl optionally substituted in the carbon skeleton by 1 or 2 methyl groups, a 1H-1,2,4-triazol-1-yl, 1H-1,2,3-triazol-1-yl, 1H-1,2,3,4-tetrazol-1-yl, a vinyl, propargyl, p-nitrophenyl, 2,4-dinitrophenyl, trichlorophenyl, pentachlorophenyl, pentafluorophenyl, pyranyl or pyridinyl, a dimethylaminyloxy, 2(1H)-oxopyridin-1-yl-oxy, 2,5-dioxopyrrolidin-1-yloxy, phthalimidyloxy, 1H-benzo-triazol-1-yloxy or azide group,

with a piperidine of general formula

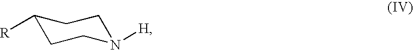

wherein

R is as hereinbefore defined.

The reaction is carried out under Schotten-Baumann or Einhorn conditions, i.e. the components are reacted in the presence of at least one equivalent of an auxiliary base at temperatures between −50° C. and +120° C., preferably −10° C. and +30° C., and optionally in the presence of solvents. The auxiliary bases used are preferably alkali metal and alkaline earth metal hydroxides, e.g. sodium hydroxide, potassium hydroxide or barium hydroxide, alkali metal carbonates, e.g. sodium carbonate, potassium carbonate or caesium carbonate, alkali metal acetates, e.g. sodium or potassium acetate, as well as tertiary amines, e.g. pyridine, 2,4,6-trimethylpyridine, quinoline, triethylamine, N-ethyl-diisopropylamine, N-ethyl-dicyclohexylamine, 1,4-diazabicyclo[2,2,2]octane or 1,8-diazabicyclo[5,4,0]undec-7-ene, the solvents used may be, for example, dichloromethane, tetrahydrofuran, 1,4-dioxane, acetonitrile, dimethyl formamide, dimethyl acetamide, N-methyl-pyrrolidone or mixtures thereof; if alkali metal or alkaline earth metal hydroxides, alkali metal carbonates or acetates are used as the auxiliary bases, water may also be added to the reaction mixture as cosolvent.

d) In order to prepare compounds of general formula (I) wherein neither X¹ nor X³ nor X⁴ nor R¹ contains a free carboxylic acid function, but otherwise all groups are as hereinbefore defined:

Coupling a carboxylic acid of general formula

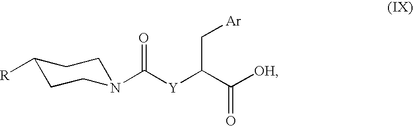

wherein

Ar, R and Y are as hereinbefore defined,

with a cyclic secondary amine of general formula

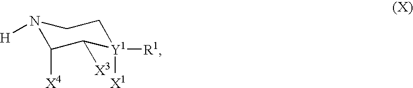

wherein

neither X¹ nor X³ nor X⁴ nor R¹ contains a free carboxylic acid function, but otherwise the groups are as hereinbefore defined.

The coupling is preferably carried out using methods known from peptide chemistry (cf. e.g. Houben-Weyl, Methoden der Organischen Chemie, Vol. 15/2), for example using carbodiimides such as e.g. dicyclohexylcarbodiimide (DCC), diisopropyl carbodiimide (DIC) or ethyl-(3-dimethylaminopropyl)-carbodiimide, O-(1H-benzotriazol-1-yl)-N,N-N′,N′-tetramethyluronium hexafluorophosphate (HBTU) or tetrafluoroborate (TBTU) or 1H-benzotriazol-1-yl-oxy-tris-(dimethylamino)-phosphonium hexafluorophosphate (BOP). By adding 1-hydroxybenzotriazole (HOBt) or 3-hydroxy-4-oxo-3,4-dihydro-1,2,3-benzotriazine (HOObt) the reaction speed can be increased. The couplings are normally carried out with equimolar amounts of the coupling components as well as the coupling reagent in solvents such as dichloromethane, tetrahydrofuran, acetonitrile, dimethyl formamide (DMF), dimethyl acetamide (DMA), N-methylpyrrolidone (NMP) or mixtures thereof and at temperatures between −30 and +30° C., preferably −20 and +25° C. If necessary, N-ethyl-diisopropylamine (DIEA) (Hünig base) is preferably used as an additional auxiliary base.

The so-called anhydride process is used as a further coupling method for synthesising compounds of general formula (I) (cf. also: M. Bodanszky, “Peptide Chemistry”, Springer-Verlag 1988, p. 58-59; M. Bodanszky, “Principles of Peptide Synthesis”, Springer-Verlag 1984, p. 21-27). The Vaughan variant of the mixed anhydride process is preferred (J. R. Vaughan Jr., J. Amer. Chem. Soc. 73, 3547 (1951)), in which the mixed anhydride of the carboxylic acid of general formula (IX) which is to be coupled and monoisobutyl carbonate is obtained, using isobutyl chlorocarbonate in the presence of bases such as 4-methyl-morpholine or 4-ethylmorpholine. The preparation of this mixed anhydride and the coupling with amines of general formula (X) are carried out in a one-pot process, using the above-mentioned solvents and at temperatures between −20 and +25° C., preferably 0° C. and +25° C.

e) In order to prepare compounds of general formula (I) wherein neither X¹ nor X³ nor X⁴ nor R¹ contains a free carboxylic acid function, but otherwise all groups are as hereinbefore defined:

Coupling a compound of general formula

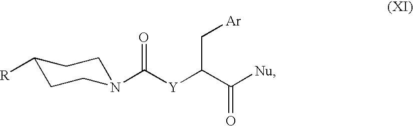

wherein

Ar, R and Y are as hereinbefore defined and Nu denotes a leaving group, for example a halogen atom, such as the chlorine, bromine or iodine atom, a C₁₋₁₀-alkylsulphonyloxy group, a phenylsulphonyloxy or naphthylsulphonyloxy group optionally mono-, di- or trisubstituted by chlorine or bromine atoms, by methyl or nitro groups, while the substituents may be identical or different, a 1H-imidazol-1-yl, a 1H-pyrazol-1-yl optionally substituted in the carbon skeleton by 1 or 2 methyl groups, a 1H-1,2,4-triazol-1-yl, 1H-1,2,3-triazol-1-yl, 1H-1,2,3,4-tetrazol-1-yl, a vinyl, propargyl, p-nitrophenyl, 2,4-dinitrophenyl, trichlorophenyl, pentachlorophenyl, pentafluorophenyl, pyranyl or pyridinyl, a dimethylaminyloxy, 2(1H)-oxopyridin-1-yl-oxy, 2,5-dioxopyrrolidin-1-yloxy, phthalimidyloxy, 1H-benzotriazol-1-yloxy or azide group,

with a cyclic secondary amine of general formula

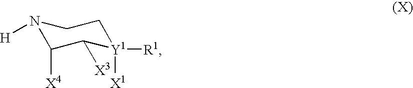

wherein

neither X¹ nor X³ nor X⁴ nor R¹ contains a free carboxylic acid function, but otherwise the groups are as hereinbefore defined.

The reaction is carried out under Schotten-Baumann or Einhorn conditions, i.e. the components are reacted in the presence of at least one equivalent of an auxiliary base at temperatures between −50° C. and +120° C., preferably −10° C. and +30° C., and optionally in the presence of solvents. The auxiliary bases used are preferably alkali metal and alkaline earth metal hydroxides, e.g. sodium hydroxide, potassium hydroxide or barium hydroxide, alkali metal carbonates, e.g. sodium carbonate, potassium carbonate or caesium carbonate, alkali metal acetates, e.g. sodium or potassium acetate, as well as tertiary amines, e.g. pyridine, 2,4,6-trimethylpyridine, quinoline, triethylamine, N-ethyl-diisopropylamine, N-ethyl-dicyclohexylamine, 1,4-diazabicyclo[2,2,2]octane or 1,8-diazabicyclo[5,4,0]undec-7-ene, the solvents used may be, for example, dichloromethane, tetrahydrofuran, 1,4-dioxane, acetonitrile, dimethyl formamide, dimethyl acetamide, N-methyl-pyrrolidone or mixtures thereof; if alkali metal or alkaline earth metal hydroxides, alkali metal carbonates or acetates are used as the auxiliary bases, water may also be added to the reaction mixture as cosolvent.

f) In order to prepare compounds of general formula (I) wherein X¹, X³, X⁴ or R¹ contains a free carboxylic acid function, but otherwise all the groups are as hereinbefore defined:

hydrolysis of carboxylic acid esters of general formula (I), wherein either X¹ or X³ or X⁴ or R¹ contains a carboxylic acid ester function and all the other groups are as hereinbefore defined. The hydrolysis may be carried out with acid or alkaline catalysis under the conditions familiar to those skilled in the art. Acid-catalysed hydrolysis takes place in the presence of strong organic or inorganic acids, for example methanesulphonic acid, p-toluenesulphonic acid, hydrochloric acid, hydrobromic acid or sulphuric acid, preferably in the presence of water-miscible solvents, for example methanol, ethanol or 1,4-dioxane, and at temperatures between 0° C. and the boiling temperature of the hydrolysis mixture. It is advantageous to carry out alkaline saponification of the carboxylic acid esters of general formula (I), optionally also in the presence of water-miscible cosolvents. To do this, at least 1 equivalent, based on the particular carboxylic acid ester, of an inorganic base such as aqueous lithium hydroxide solution, sodium, potassium or barium hydroxide solution is used. Suitable temperatures are between 0° C. and 50° C., room temperature being preferred. The desired acid can be released from the salt initially obtained by acidification in known manner.

The new carboxylic acids and carboxylic acid esters of general formula (I) according to the invention contain one or more chiral centres. If for example there are two chiral centres the compounds may occur in the form of two pairs of diastereomeric antipodes. The invention covers the individual isomers as well as the mixtures thereof.

The diastereomers may be separated on the basis of their different physico-chemical properties, e.g. by fractional crystallisation from suitable solvents, by high pressure liquid or column chromatography, using chiral or preferably non-chiral stationary phases.

Racemates covered by general formula (I) may be separated for example by HPLC on suitable chiral stationary phases (e.g. Chiral AGP, Chiralpak AD). Racemates which contain a basic or acidic function can also be separated via the diastereomeric, optically active salts which are produced on reacting with an optically active acid, for example (+) or (−)-tartaric acid, (+) or (−)-diacetyl tartaric acid, (+) or (−)-monomethyl tartrate or (+)-camphorsulphonic acid, or an optically active base, for example with (R)-(+)-1-phenylethylamine, (S)-(−)-1-phenylethylamine or (S)-brucine.

According to a conventional method of separating isomers, the racemate of a compound of general formula (I) is reacted with one of the above-mentioned optically active acids or bases in equimolar amounts in a solvent and the resulting crystalline, diastereomeric, optically active salts thereof are separated using their different solubilities. This reaction may be carried out in any type of solvent provided that it is shows a sufficient difference in terms of the solubility of the salts. Preferably, methanol, ethanol or mixtures thereof, for example in a ratio by volume of 50:50, are used. Then each of the optically active salts is dissolved in water, carefully neutralised with a base such as sodium carbonate or potassium carbonate, or with a suitable acid, e.g. dilute hydrochloric acid or aqueous methanesulphonic acid, and in this way the corresponding free compound is obtained in the (+) or (−) form.

The (R) or (S) enantiomer alone or a mixture of two optically active diastereomeric compounds covered by general formula I may also be obtained by performing the syntheses described above with a suitable reaction component in the (R) or (S) configuration.

The starting compounds of general formula (IV) may be obtained, if they are not known from the literature or even commercially available, according to the processes described in WO 98/11128 and DE 199 52 146. The starting compounds of general formula (V) are commercially available. Compounds of general formula (VI) may be obtained by methods familiar to the peptide chemist from protected phenylalanines and amines of general formula (X). The starting compounds of general formula (VII) are obtained for example by reacting cyclic secondary amines of general formula (X) with 2-(alkoxycarbonylmethyl)-3-aryl-propanoic acids and subsequently hydrolytically cleaving the alkyl group. The 2-(alkoxycarbonylmethyl)-3-aryl-propanoic acids required may be prepared analogously to methods known from the literature (Saul G. Cohen and Aleksander Milovanovic, J. Am. Chem. Soc. 90, 3495-3502 [1968]; Hiroyuki Kawano, Youichi Ishii, Takao lkariya, Masahiko Saburi, Sadao Yoshikawa, Yasuzo Uchida and Hidenori Kumobayashi, Tetrahedron Letters 28, 1905-8 [1987]). Carboxylic acids of general formula IX have been described in WO 98/11128 or may be prepared using the methods described therein from generally available starting materials. The cyclic secondary amines of general formula (X) may be synthesised from compounds of general formula

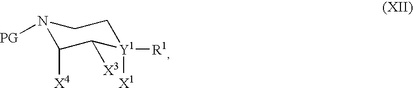

wherein PG denotes a cleavable protective group, for example by hydrogenolysis of a phenylmethyl group. The preliminary products for synthesising the compounds of general formula (XII) are obtainable from starting materials which are commercially available or easily obtained by common methods. Finally, the starting compounds of general formulae VIII and XI may be prepared from the corresponding carboxylic acids (VII) or (IX) using known standard methods.

The compounds of general formula I obtained may, if they contain suitable basic functions, be converted, particularly for pharmaceutical use, into their physiologically acceptable salts with inorganic or organic acids. Suitable acids include for example hydrochloric acid, hydrobromic acid, phosphoric acid, nitric acid, sulphuric acid, methanesulphonic acid, ethanesulphonic acid, benzenesulphonic acid, p-toluenesulphonic acid, acetic acid, fumaric acid, succinic acid, lactic acid, mandelic acid, malic acid, citric acid, tartaric acid or maleic acid.

Moreover, the new compounds of formula (I), if they contain a carboxylic acid function, may if desired be converted into the addition salts thereof with inorganic or organic bases, particularly for pharmaceutical use into the physiologically acceptable addition salts thereof. Suitable bases for this include, for example, sodium hydroxide, potassium hydroxide, ammonia, cyclohexylamine, dicyclohexylamine, ethanolamine, diethanolamine and triethanolamine.

The new compounds of general formula I and the physiologically acceptable salts thereof have CGRP-antagonistic properties and exhibit good affinities in CGRP receptor binding studies. The compounds display CGRP-antagonistic properties in the pharmacological test systems described hereinafter.

The following experiments were carried out to demonstrate the affinity of the compounds of general formula I for human CGRP-receptors and their antagonistic properties:

A. Binding Studies With SK-N-MC Cells (Expressing the Human CGRP Receptor)

SK-N-MC cells are cultivated in “Dulbecco's modified Eagle medium”. The medium is removed from confluent cultures. The cells are washed twice with PBS buffer (Gibco 041-04190 M), detached by the addition of PBS buffer mixed with 0.02% EDTA, and isolated by centrifuging. After resuspension in 20 ml of “Balanced Salts Solution” [BSS (in mM): NaCl 120, KCl 5.4, NaHCO₃ 16.2, MgSO₄ 0.8, NaHPO₄ 1.0, CaCl₂ 1.8, D-glucose 5.5, HEPES 30, pH 7.40] the cells are centrifuged twice at 100×g and resuspended in BSS. After the number of cells has been determined, the cells are homogenised using an Ultra-Turrax and centrifuged for 10 minutes at 3000×g. The supernatant is discarded and the pellet is recentrifuged in Tris buffer (10 mM Tris, 50 mM NaCl, 5 mM MgCl₂, 1 mM EDTA, pH 7.40 enriched with 1% bovine serum albumin and 0.1% bacitracin), and resuspended (1 ml/1000000 cells). The homogenised product is frozen at −80° C. The membrane preparations are stable for more than 6 weeks under these conditions.

After thawing, the homogenised product is diluted 1:10 with assay buffer (50 mM Tris, 150 mM NaCl, 5 mM MgCl₂, 1 mM EDTA, pH 7.40) and homogenised for 30 seconds with an Ultra-Turrax. 230 μl of the homogenised product are incubated for 180 minutes at ambient temperature with 50 pM ¹²⁵I-iodotyrosyl-Calcitonin-Gene-Related Peptide (Amersham) and increasing concentrations of the test substances in a total volume of 250 μl. The incubation is ended by rapid filtration through GF/B-glass fibre filters treated with polyethyleneimine (0.1%) using a cell harvester. The protein-bound radioactivity is measured using a gamma counter. Non-specific binding is defined as the bound radioactivity in the presence of 1 μM human CGRP-alpha during incubation.

The concentration binding curves are analysed using computer-aided non-linear curve matching.

The compounds of general formula (I) show IC₅₀ values ≦10000 nM in the test described.

B. CGRP Antagonism in SK-N-MC Cells

SK-N-MC cells (1 million cells) are washed twice with 250 μl incubation buffer (Hanks' HEPES, 1 mM 3-isobutyl-1-methylxanthine, 1% BSA, pH 7.4) and pre-incubated at 37° C. for 15 minutes. After the addition of CGRP (10 μl) as agonist in increasing concentrations (10⁻¹¹ to 10⁻⁶ M), or additionally the substance in 3 to 4 different concentrations, the mixture is incubated for another 15 minutes.

Intracellular cAMP is then extracted by the addition of 20 μl of 1 M HCl and centrifugation (2000×g, 4° C., for 15 minutes). The supernatants are frozen in liquid nitrogen and stored at −20° C.

The cAMP contents of the samples are determined by radioimmunoassay (Messrs. Amersham) and the pA₂ values of antagonistically acting substances are determined graphically.

The compounds of general formula (I) exhibit CGRP-antagonistic properties in the in vitro test model described, in a dosage range between 10⁻¹¹ and 10⁻⁵ M.

In view of their pharmacological properties the compounds of general formula I and the salts thereof with physiologically acceptable acids are thus suitable for the acute and prophylactic treatment of headaches, particularly migraine or cluster headaches. Moreover, the compounds of general formula I also have a positive effect on the following diseases: “complex regional pain syndrome”, non-insulin-dependent diabetes mellitus (“NIDDM”), cardiovascular diseases, morphine tolerance, diarrhoea caused by clostridium toxin, skin diseases, particularly thermal and radiation-induced skin damage including sunburn, inflammatory diseases, e.g. inflammatory diseases of the joints (arthritis), inflammatory lung diseases, allergic rhinitis, asthma, diseases accompanied by excessive vasodilatation and resultant reduced blood supply to the tissues, e.g. shock and sepsis. The symptoms of menopausal hot flushes caused by vasodilatation and increased blood flow in oestrogen-deficient women are favourably affected by the CGRP-antagonists of the present application in a preventive and acute-therapeutic capacity, this therapeutic approach being distinguished from hormone replacement by the absence of side effects. In addition, the compounds according to the invention have a general pain-relieving effect.

The dosage required to achieve a corresponding effect is conveniently 0.001 to 30 mg/kg of body weight, preferably 0.01 to 5 mg/kg of body weight, when administered intravenously or subcutaneously, and 0.01 to 50 mg/kg of body weight, preferably 0.1 to 30 mg/kg of body weight when administered orally, nasally or by inhalation, 1 to 3× a day in each case.

For this purpose, the compounds of general formula I prepared according to the invention may be formulated with other active substances such as e.g. antiemetics, prokinetics, neuroleptics, antidepressants, neurokinine antagonists, anticonvulsants, histamine-H1 receptor antagonists, antimuscarinics, β-blockers, α-agonists and α-antagonists, ergot alkaloids, mild analgesics, non-steroidal antiinflammatories, corticosteroids, calcium antagonists, 5-HT_{1D} agonists or other anti-migraine agents, together with one or more inert conventional carriers and/or diluents, e.g. with corn starch, lactose, glucose, microcrystalline cellulose, magnesium stearate, polyvinyl pyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, into conventional galenic preparations such as plain or coated tablets, capsules, powders, suspensions, solutions, metered dose aerosols or suppositories.

Thus other active substances which may be used for the combinations mentioned above include for example meloxicam, ergotamine, dihydroergotamine, metoclopramide, domperidone, diphenhydramine, cyclizine, promethazine, chlorpromazine, dexamethasone, flunarizine, dextropropoxyphene, meperidine, propranolol, nadolol, atenolol, clonidine, indoramin, carbamazepine, phenyloin, valproate, amitryptilin, lidocaine, diltiazem or sumatriptan and other 5-HT_{1D} agonists such as e.g. naratriptan, zolmitriptan, avitriptan, rizatriptan and eletriptan. The dosage for these active substances is expediently ⅕ of the lowest usually recommended dose to 1/1 of the normally recommended dose, i.e. for example 20 to 100 mg of sumatriptan.

The invention further relates to the use of the compounds of general formula (I) as valuable adjuvants for the production and purification (by affinity chromatography) of antibodies as well as in RIA and ELISA assays, after suitable radioactive labelling, for example by direct labelling with ¹²⁵I or ¹³¹I or by tritiation of suitable precursors, for example by replacing halogen atoms with tritium, and as a diagnostic or analytical aid in neurotransmitter research.

The Examples that follow are intended to illustrate the invention more fully:

Preliminary Remarks:

The compounds were prepared in some cases by conventional methods of synthesis and in other cases using methods of combined chemistry.

The automatic synthesiser used was the ASW2000 machine made by Chemspeed Ltd., Rheinstraβe 32, CH-4302 Augst, Switzerland.

As a rule, IR, ¹H-NMR and/or mass spectra have been obtained for all the compounds prepared by conventional methods. Unless otherwise stated, R_{f} values were obtained using ready-made silica gel TLC plates 60 F254 (E. Merck, Darmstadt, Item no.1.05714) without chamber saturation. If no detailed information is given as to the configuration, it is not clear whether it is a pure enantiomer or whether partial or even complete racemisation has occurred. The following eluants or eluant mixtures were used for the chromatography:

EI A=ethyl acetate/methanol 100/5 v/v

EI B=ethyl acetate/methanol 9/1 v/v

EI C=ethyl acetate/methanol/conc. ammonia 80/20/1 v/v/v

EI D=dichloromethane/cyclohexane/methanol/conc.ammonia 70/15/15/2 v/v/v/v

EI E=ethyl acetate/glacial acetic acid 99/1 v/v

EI F=ethyl acetate/methanol/glacial acetic acid 90/10/1 v/v/v

EI G=dichloromethane/methanol/conc. ammonia 90/9/1 v/v/v

EI H=petroleum ether/ethyl acetate 4/6 v/v

EI I=dichloromethane/methanol/glacial acetic acid 90/10/2.5 v/v/v

EI K=dichloromethane/isopropanol 9/1 v/v

EI M=dichloromethane/methanol/conc. ammonia 75/25/5 v/v/v

EI N=dichloromethane/ethyl acetate 1/1 v/v

EI 0=dichloromethane/methanol 9/1 v/v

EI P=dichloromethane/ethyl acetate/cyclohexane/methanol/conc. ammonia 60/16/5/5/0.6 v/v/v/v/v

EI Q=dichloromethane/methanol/conc. ammonia 80/20/2 v/v/v

EI R=dichloromethane/methanol/glacial acetic acid 80/20/1 v/v/v

EI S=dichloromethane/methanol 9/1 v/v (Alox TLC plates [E. Merck, Darmstadt])

EI T=dichloromethane/methanol/glacial acetic acid 70/30/3 v/v/v

EI U=ethyl acetate/petroleum ether 2/1 v/v

EI V=ethyl acetate/petroleum ether 1/4 v/v

EI W=ethyl acetate/petroleum ether 3/7 v/v

EI X=petroleum ether/ethyl acetate/glacial acetic acid 8/2/0.5 v/v/v

EI Y=ethyl acetate/petroleum ether 1/9 v/v

EI Z=toluene/petroleum ether/ethyl acetate 5/5/2 v/v/v

EI AA=ethyl acetate/petroleum ether/triethylamine 5/5/0.1 v/v/v

EI BB=dichloromethane/methanol 3/1 v/v (Alox TLC plates [E. Merck, Darmstadt])

EI DD=ethyl acetate/methanol/conc. ammonia 70/30/3 v/v/v

EI EE=dichloromethane/ethanol 9/1 v/v

EI FF=dichloromethane/ethanol 50/1 v/v

EI GG=dichloromethane/ethanol 40/1 v/v

EI HH=dichloromethane/methanol 5/1 v/v

EI II=ethyl acetate/methanol/conc. ammonia 90/10/1 v/v/v

EI KK=ethyl acetate/methanol/conc. ammonia 60/40/4 v/v/v

EI LL=ethyl acetate/methanol/conc. ammonia 50/50/5 v/v/v

EI MM=ethyl acetate/cyclohexane 1/1 v/v

EI NN=ethyl acetate/cyclohexane 2/8 v/v

EI OO=dichloromethane/methanol/conc. ammonia 70/30/3 v/v/v

The following abbreviations are used in the description of the test:

mp.: melting point

(Z): (decomposition)

DIEA: N,N-diisopropylethylamine

Boc: (1,1-dimethylethoxy)carbonyl

TBTU: 2-(1H-benzotriazol-1-yl)-1,1,3,3-tetramethyluronium tetrafluoroborate

HOBt: 1-hydroxybenzotriazole-hydrate

CDT: 1,1′-carbonyldi-(1,2,4-triazole)

PyBroP: bromo-tris-pyrrolidino-phosphonium hexafluorophosphate

HATU: O-(7-azabenzotriazol-1-yl)-N,N,N′,N′-tetramethyluronium hexafluoro-phosphate

THF: tetrahydrofuran

DMF: dimethylformamide

EE: ethyl acetate

PE: petroleum ether

LM: solvent

ZT room temperature

Ser. no: serial no.

The meanings of the symbols consisting of letters and numbers used in the Examples are shown in the following summary:

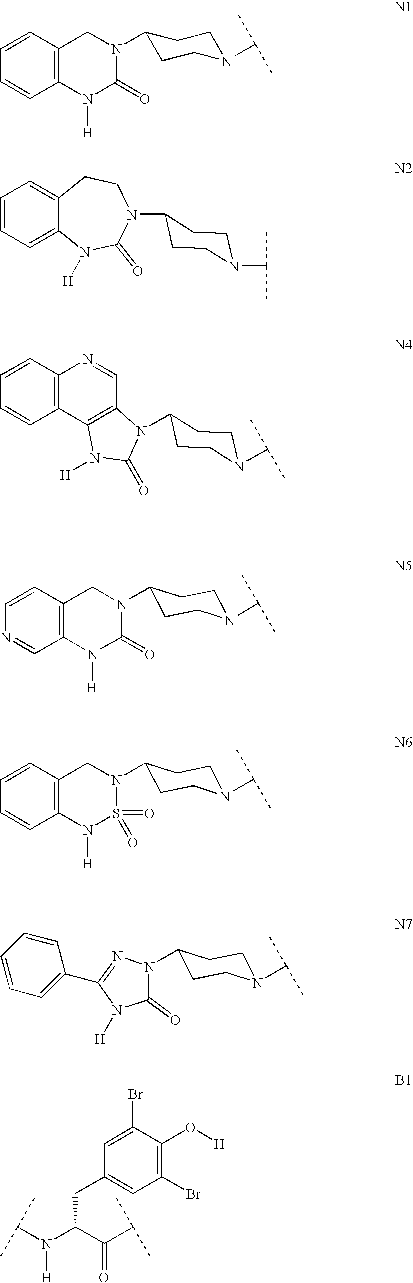

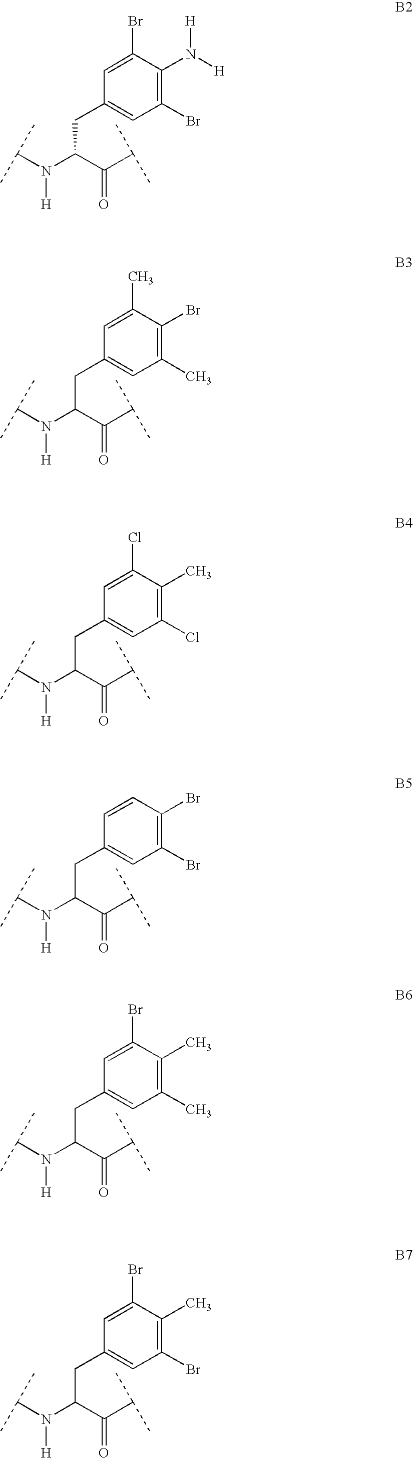

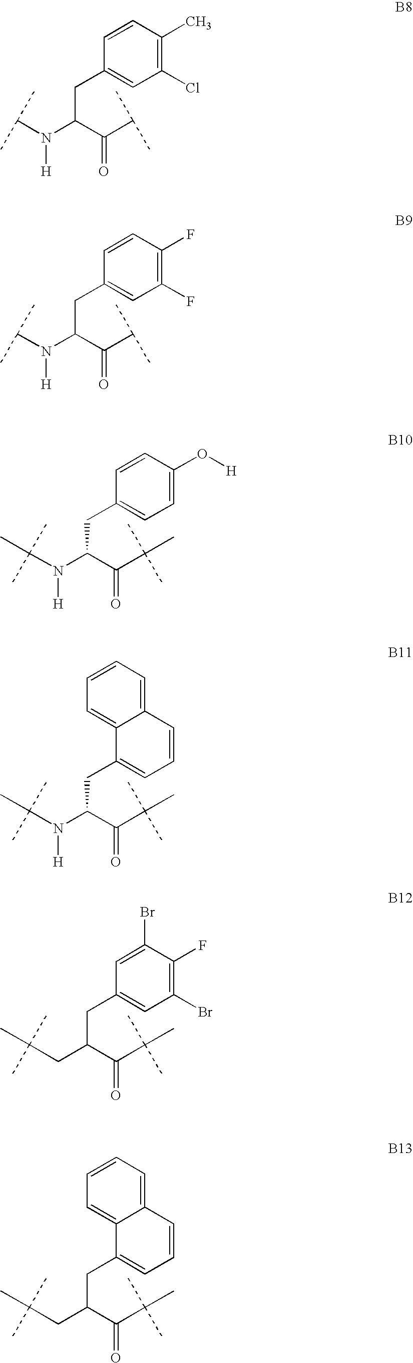

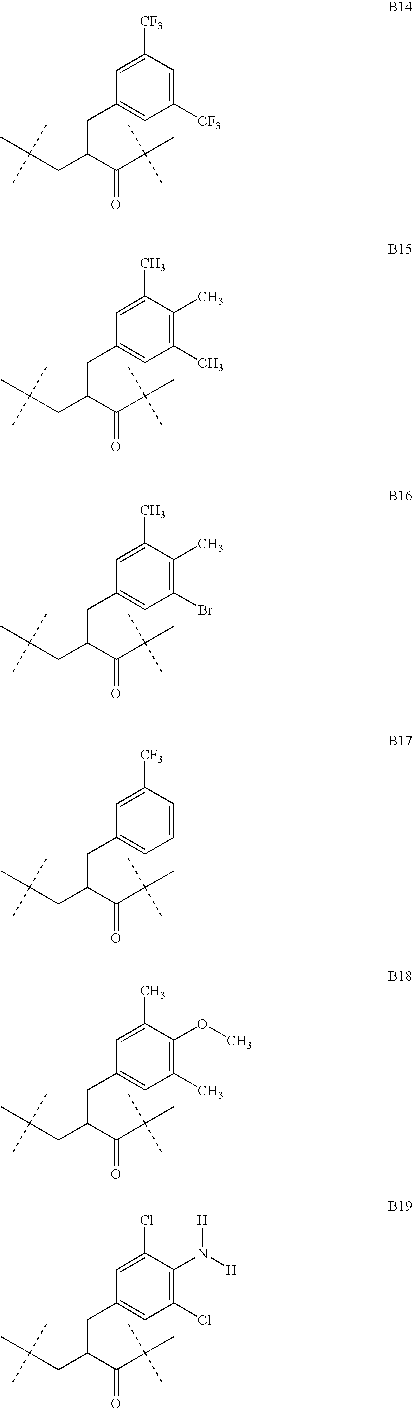

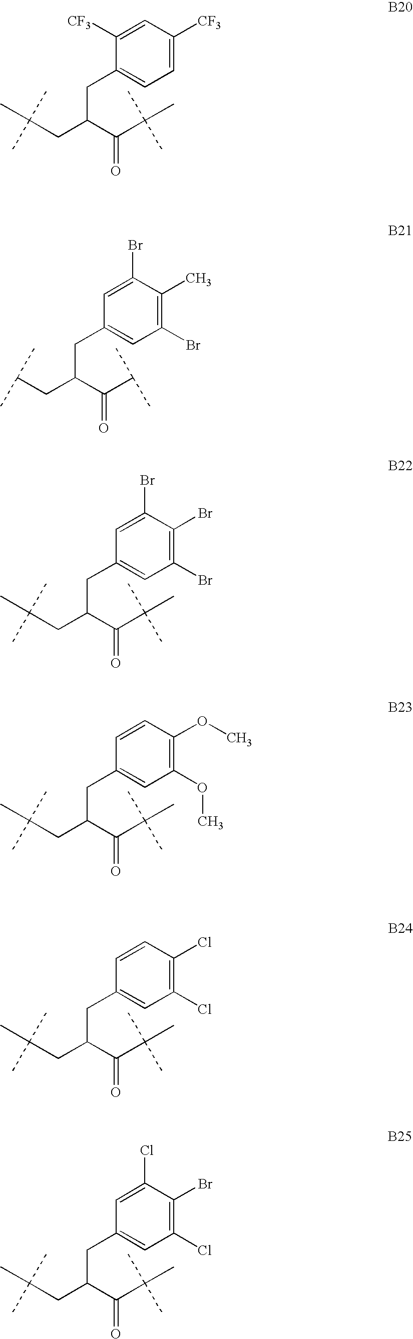

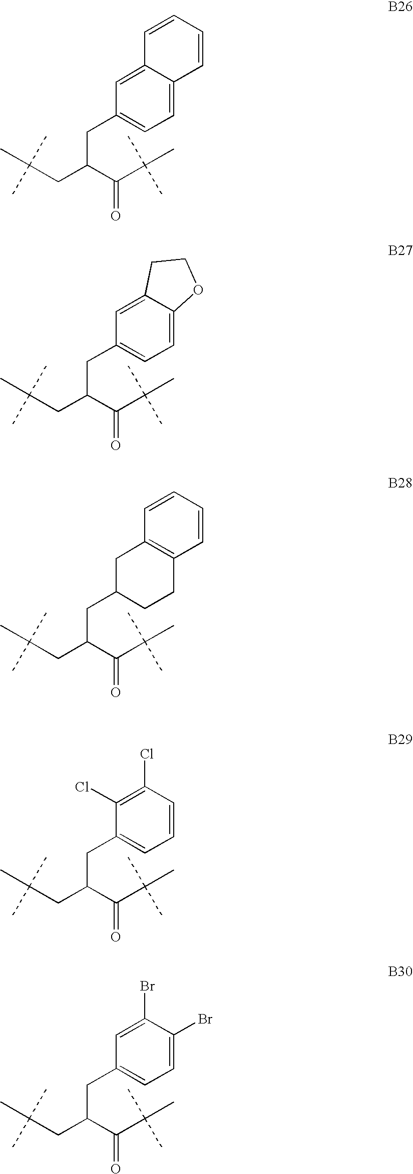

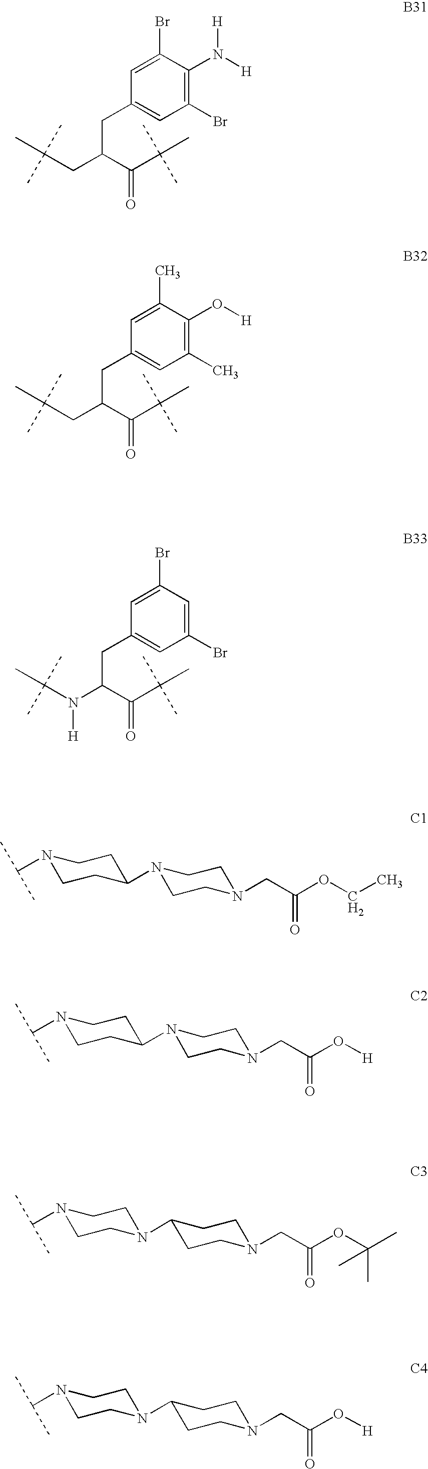

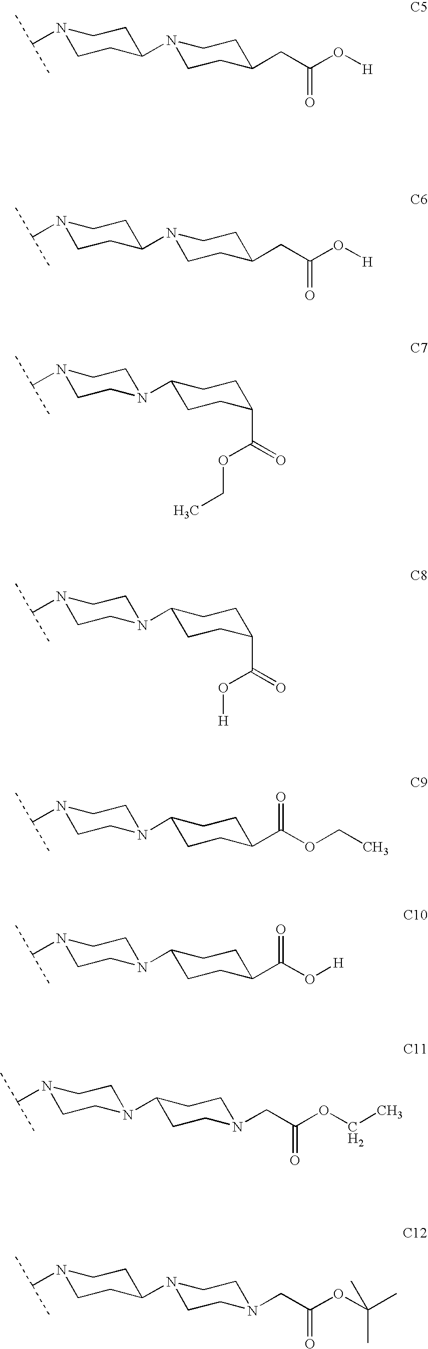

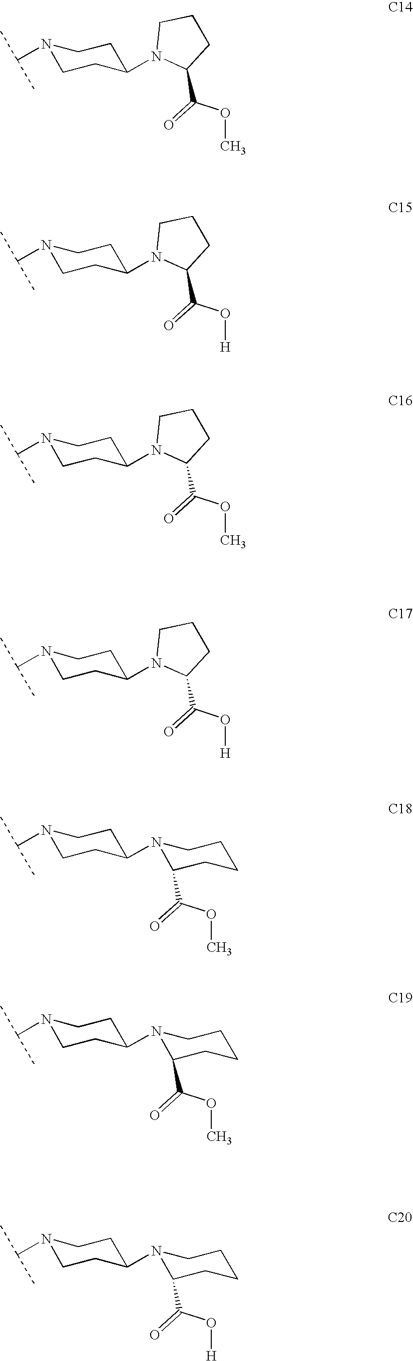

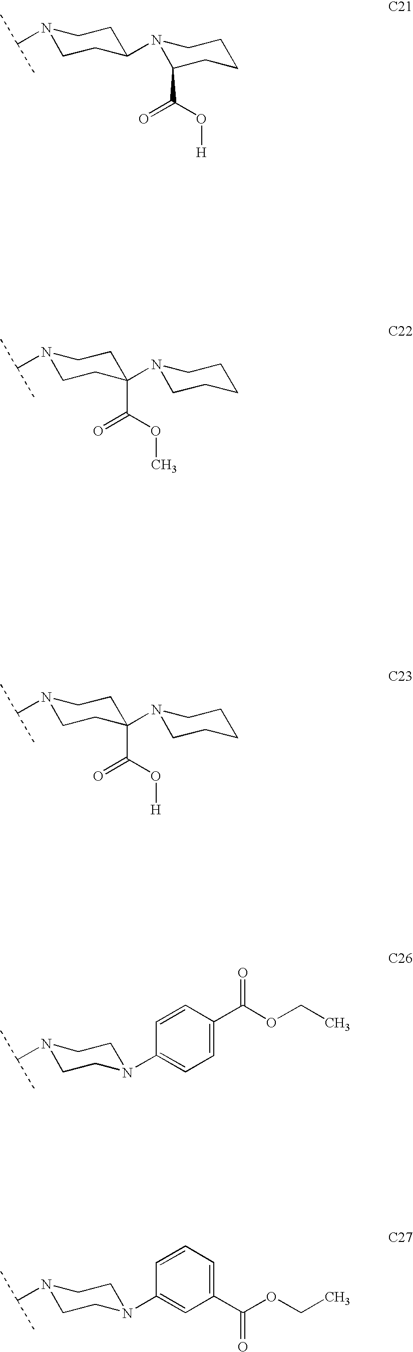

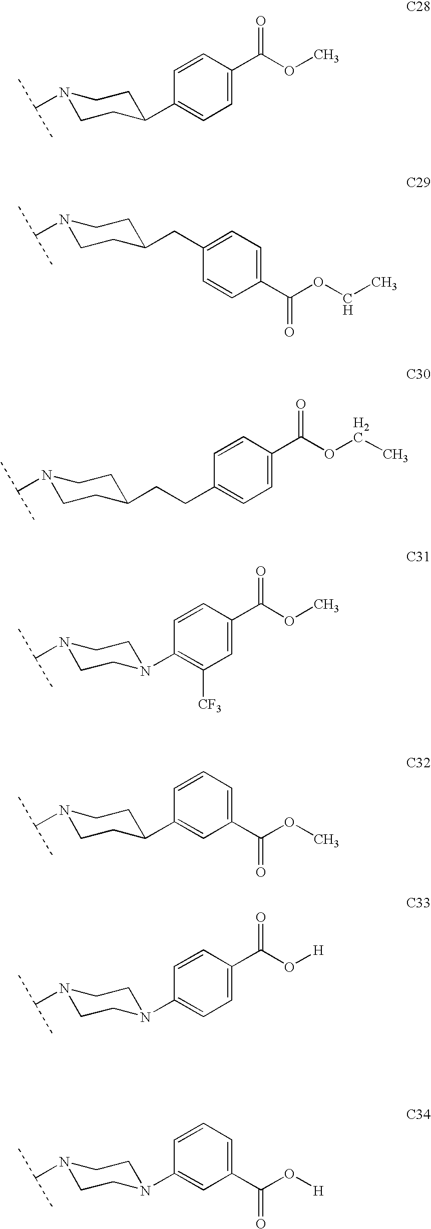

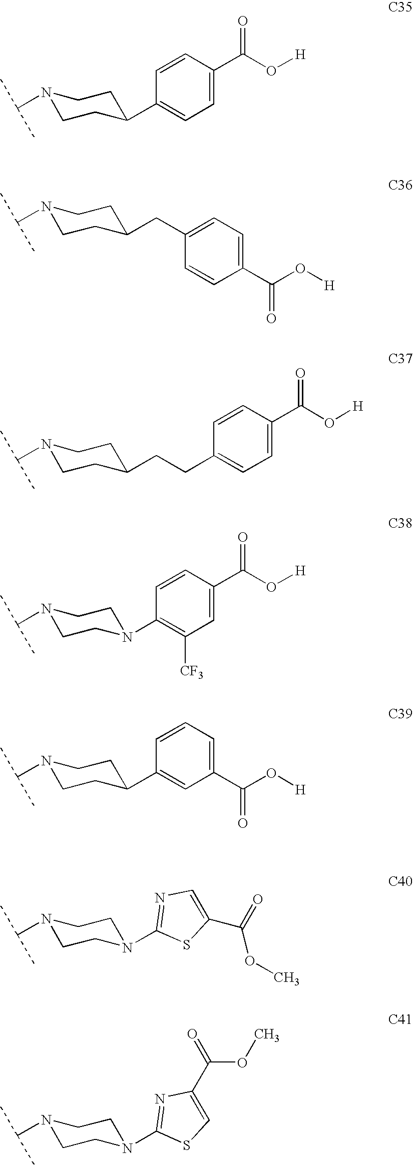

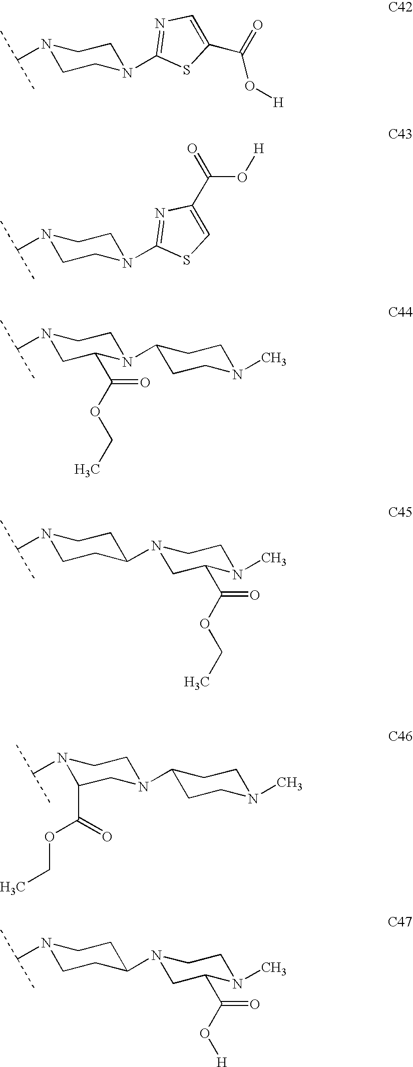

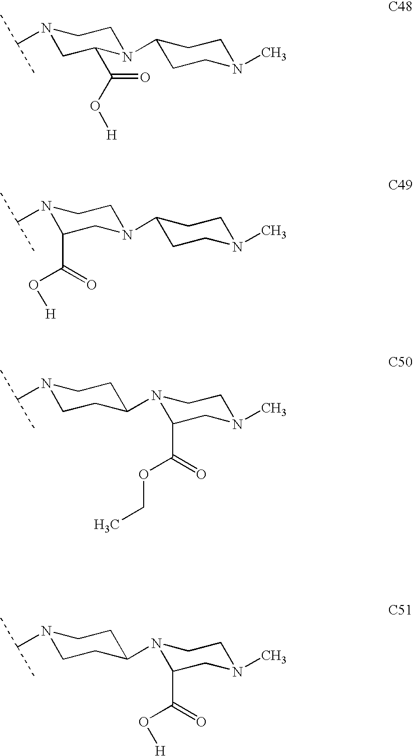

A. Preparation of Intermediate Compounds

### EXAMPLE A1

(R,S)-3,4-dibromo-N-[[4-(3,4-dihydro-2(1H)-oxoquinazolin-3-yl)-1-piperidinyl]-carbonyl]-phenylalanine

150 ml 1M sodium hydroxide solution were added to the solution of 20.0 g (0.033 mol) (R,S)-3,4-dibromo-N-[[4-(3,4-dihydro-2(1H)-oxoquinazolin-3-yl)-1-piperidinyl]carbonyl]-phenylalanine ethyl ester in 500 ml of ethanol and the mixture was then stirred for 3.5 hours at room temperature. The solvent was eliminated using the rotary evaporator and the residue was acidified with 1 M hydrochloric acid. The precipitated precipitate was suction filtered, washed thoroughly with water and dried at 70° C. in the circulating air dryer. 10.0 g (52% of theory) of the desired colourless crystalline substance were obtained, R_{f} 0.62 (EI M).

IR (KBr): 1705, 1645 cm⁻¹ (C═O)

The following compounds of general formula N-B-C were prepared analogously:

| N | B | C | Remarks | % yield | EI | R_{f} | MS | IR [cm⁻¹] | mp. [° C.] |
|---|---|---|---|---|---|---|---|---|---|
| N1 | B6 | OH | from N1—CO—B6—OEt | 96 | | | ESI: (M − H)⁻ = 527/ | 1630, 1701 | 173-175 |
| | | | with aq. 1M NaOH, then | | | | 529 (Br) | (C═O) | |
| | | | aq. 1M HCl | | | | | | |
| N1 | B7 | OH | from N1—CO—B7—OEt | 62 | D | 0.19 | | 1705 | colourless |
| | | | with aq. 1M NaOH, then | | | | | (C═O) | crystals |
| | | | aq. 1M HCl | | | | | | |
| N1 | B10 | OH | from N1—CO—B10—OMe | 79 | | | ESI: (M + Na)⁺ = 481 | | colourless |
| | | | with aq. 1M NaOH, then | | | | | | crystals |
| | | | aq. 1M HCl | | | | | | |
| N1 | B11 | OH | from N1—CO—B11—OMe | 61 | | | ESI: (M + H)⁺ = 439 | | colourless |
| | | | with aq. 1M LiOH, then | | | | | | crystals |
| | | | aq. citric acid | | | | | | |
| N1 | B3 | OH | from N1—CO—B3—OEt | 95 | | | | | colourless |
| | | | with aq. 1M LiOH, then | | | | | | crystals |
| | | | aq. citric acid | | | | | | |
| N1 | B4 | OH | from N1—CO—B4—OEt | 96 | B | 0.12 | ESI: (M − H)⁻ = 503/ | | colourless |
| | | | with aq. 1M NaOH, then | | | | 505/507 | | crystals |
| | | | aq. 1M HCl | | | | (Cl₂) | | |
| N1 | B12 | OH | from N1—CO—B12[α- | 100 | G | 0.11 | ESI: (M − H)⁻ = 594/ | | colourless |
| | | | CO₂Et]-OEt with aq. | | | | 596/598 | | crystals |
| | | | 40% NaOH, then aq. 5M | | | | (Br₂) | | |
| | | | HCl | | | | | | |
| N1 | B15 | OH | from N1—CO—B15[α- | 46 | F | 0.60 | ESI: (M − H)⁻ = 462; | 1647 | colourless |
| | | | CO₂Et]-OEt with aq. 1M | | | | (M + H)⁺ = 464 | (C═O) | crystals |
| | | | NaOH, then aq. 1M HCl | | | | | | |
| N1 | B16 | OH | from N1—CO—B16[α- | 100 | F | 0.49 | ESI: (M − H)⁻ = 526 | 1645 | colourless |
| | | | CO₂Et]-OEt with aq. 1M | | | | | (C═O) | crystals |
| | | | NaOH, then aq. 1M HCl | | | | | | |
| N1 | B19 | OH | from N1—CO—B19[α- | 50 | | | | | colourless |
| | | | CO₂Et]-OEt with aq. 1M | | | | | | crystals |
| | | | NaOH, then aq. 1M HCl | | | | | | |
| N1 | B20 | OH | from N1—CO—B20[α- | 55 | D | 0.23 | M⁺ = 557; ESI: | | colourless |
| | | | CO₂Et]-OEt with aq. 1M | | | | (M − H)⁻ = 556 | | crystals |
| | | | NaOH, then aq. 1M HCl | | | | | | |
| N1 | B22 | OH | from N1—CO—B22[α- | 91 | D | 0.25 | ESI: (M − H)⁻ = 654/ | 1641 | colourless |
| | | | CO₂Et]-OEt with aq. 1M | | | | 656/658/660 | (C═O) | crystals |
| | | | NaOH, then aq. 1M HCl | | | | (Br₃) | | |
| N1 | B25 | OH | from N1—CO—B25[α- | 62 | F | 0.4 | no M⁺, | 1726, | colourless |
| | | | CO₂Et]-OEt with aq. 1M | | | | decomposition | 1705, 1641 | crystals |
| | | | KOH, then aq. 1M HCl | | | | compatible | (C═O) | |
| | | | | | | | with structure | | |
| N1 | B27 | OH | from N1—CO—B27[α- | 87 | F | 0.55 | | | colourless |
| | | | CO₂Et]-OEt with aq. 1M | | | | | | crystals |
| | | | NaOH, then aq. 1M HCl | | | | | | |
| N1 | B29 | OH | from N1—CO—B29[α- | 100 | D | 0.46 | no M⁺, | 1640 | colourless |
| | | | CO₂Et]-OEt with KOH, | | | | decomposition | (C═O) | crystals |
| | | | then aq. 10M HCl | | | | compatible | | |
| | | | | | | | with structure | | |
| N1 | B21 | OH | from N1—CO—B21[α- | 71 | D | 0.16 | no M⁺, | 1724, 1643 | colourless |
| | | | CO₂Et]-OEt with 1M | | | | decomposition | (C═O) | crystals |
| | | | NaOH, then aq. 1M HCl | | | | compatible | | |
| | | | | | | | with structure | | |
| N1 | B8 | OH | from N1—CO—B8—OEt | 90 | Q | 0.23 | | 1730, 1665 | colourless |
| | | | with 1M NaOH, then aq. | | | | | (C═O) | crystals |
| | | | 1M HCl | | | | | | |
| N1 | B30 | OH | from N1—CO—B30[α- | 100 | F | 0.45 | ESI: (M − H)⁻ = 576/ | | colourless |
| | | | CO₂Et]-OEt with 1M | | | | 578/580 | | crystals |
| | | | NaOH, then aq. 1M HCl | | | | (Br₂) | | |
| N1 | B23 | OH | from N1—CO—B23—OMe | 96 | | | | | |
| | | | with 1M NaOH, then aq. | | | | | | |
| | | | 1M HCl | | | | | | |
| N1 | B24 | OH | from N1—CO—B24[α- | 98 | F | 0.29 | | | colourless |
| | | | CO₂Et]-OEt with 1M | | | | | | crystals |
| | | | NaOH, then aq. 1M HCl | | | | | | |
| N6 | B21 | OH | from N6—CO—B21[α- | 89 | | | ESI: (M − H)⁻ = 626/ | | colourless |
| | | | CO₂Et]-OEt with 1M | | | | 628/630 | | crystals |
| | | | NaOH, then aq. 1M HCl | | | | (Br₂) | | |
| N2 | B2 | OH | from N2—CO—B2—OMe | 96 | M | 0.49 | ESI: (M − H)⁻ = 606/ | 1724, 1660 | colourless |
| | | | with 1M LiOH, then aq. | | | | 608/610 | (C═O) | crystals |
| | | | 1M HCl | | | | (Br₂) | | |

### EXAMPLE A2

3,4-dibromo-N-[[4-(3,4-dihydro-2(1H)-oxoquinazolin-3-yl)-1-piperidinyl]carbonyl]-D,L-phenylalanine ethyl ester

9.7 g (0.056 Mol) CDT were added to an ice-cooled suspension of 18.0 g (0.051 Mol) (R,S)-3,4-dibromo-phenylalanine ethyl ester in 300 ml THF. The reaction mixture was then stirred for 1 hour at 0° C. and 1 hour at ambient temperature and then combined with 11.9 g (0.051 mol) 3-(4-piperidinyl)-1,3,4,5-tetrahydro-1,3-benzodiazepin-2-one. The mixture was refluxed for 4 hours and left to stand overnight at ambient temperature. The reaction mixture was concentrated by evaporation using the rotary evaporator, the residue was combined with 300 ml aqueous sodium hydrogen carbonate solution and stirred for 30 minutes. The aqueous solution was decanted off, the residue was combined with 150 ml of ethanol and refluxed. After cooling the white solid obtained was suction filtered, washed with ethanol and dried at 50° C. 20.0 g (64% of theory) of the product were obtained, with an R_{f} value of 0.68 (EI D).

IR (KBr): 1734, 1680,1662 (C═O) cm⁻¹

The following compounds of general formula N-B-C were prepared analogously:

| N | B | C | Remarks | % yield | EI | R_{f} | MS | IR [cm⁻¹] | mp. [° C.] |
|---|---|---|---|---|---|---|---|---|---|
| N1 | B6 | OEt | from N1—H, CDT and H— | 90 | B | 0.67 | M⁺ = 557 | 1732, 1662 | colourless |
| | | | B6—OEt in THF | | | | | (C═O) | crystals |
| N1 | B7 | OEt | from N1—H, CDT and H— | 100 | D | 0.45 | | | colourless |
| | | | B7—OEt in THF | | | | | | crystals |
| N1 | B11 | OMe | from N1—H, CDT, H—B11— | 97 | | | ESI: | | |
| | | | OMe * HCl and DIEA in | | | | (M − H)⁻ = 471 | | |
| | | | THF | | | | | | |
| N1 | B10 | OMe | from N1—H, CDT, H—B10— | 63 | G | 0.55 | ESI: | | |
| | | | OMe * HCl and DIEA in | | | | (M + H)⁺ = 453 | | |
| | | | THF | | | | | | |
| N1 | B3 | OEt | from N1—H, CDT, H—B3— | 92 | | | | 1739, | colourless |
| | | | OEt * HCl and NEt₃ in | | | | | 1682, 1664 | crystals |
| | | | THF/DMF 2/1 v/v | | | | | (C═O) | |
| N1 | B4 | OEt | from N1—H, CDT and H— | 73 | B | 0.50 | ESI: | 3402 (NH); | 200-202 |
| | | | B4—OEt in THF | | | | (M + H)⁺ = 533 | 1741, | |
| | | | | | | | | 1680, 1662 | |
| | | | | | | | | (C═O) | |
| N1 | B8 | OEt | from N1—H, CDT and H— | 72 | | | M⁺ = 498/500 | 1736, 1664 | colourless |
| | | | B8—OEt in THF | | | | (Cl) | (C═O) | crystals |
| N2 | B2 | OMe | from N2—H, CDT and H— | 96 | D | 0.76 | ESI: (M − H)⁻ = 620/ | 1728, 1664 | colourless |
| | | | B2—OMe * HCl and DIEA | | | | 622/624 | (C═O) | crystals |
| | | | in THF | | | | (Br₂); | | |
| | | | | | | | (M + Na)⁺ = 644/ | | |
| | | | | | | | 646/ | | |
| | | | | | | | 648 (Br₂) | | |

### EXAMPLE A3

Ethyl 2-[(3,5-dibromo-4-fluoro-phenyl)methyl]-4-[4-(3,4-dihydro-2(1H)-oxoquinazolin-3-yl)-1-piperidinyl]-2-(ethoxycarbonyl)-4-oxobutanoate

The mixture of 4.39 g (0.019 mol) 3,4-dihydro-3-(4-piperidinyl)-2(1H)-quinazolinone, 9.25 g (0.019 mol) β,β-bis-(ethoxycarbonyl)-3,5-dibromo-4-fluoro-benzenebutanoic acid, 6.08 g (0.019 mol) TBTU, 6.9 ml (0.05 mol) triethylamine, 200 ml THF and 70 ml DMF was stirred overnight at room temperature. The solvents were eliminated in vacuo and the residue combined with dichloromethane and 10% aqueous citric acid solution. The organic phase was separated off, extracted with sodium hydrogen carbonate solution and dried over sodium sulphate. After elimination of the desiccant and solvent the residue was combined with tert-butylmethylether and the precipitated solid substance was suction filtered. 11.0 g (83% of theory) of the desired product were obtained, mp=167-170°.

IR (KBr): 1734,1662 (C═O) cm⁻¹

ESI-MS: (M+H)+696/698/700 (Br₂)

The following compounds of general formula N-B-C were prepared analogously:

| N | B | C | Remarks | % yield | EI | R_{f} | MS | IR [cm⁻¹] | mp. [° C.] |
|---|---|---|---|---|---|---|---|---|---|
| N1 | B15[α- | OEt | from N1—H, | 89 | AcOEt | 0.7 | | 1734, | colourless |
| | CO₂Et] | | HO₂C—B15[α- | | | | | 1666 | crystals |
| | | | CO₂Et]-OEt, | | | | | (C═O) | |
| | | | TBTU, HOBt and | | | | | | |
| | | | NEt₃ in | | | | | | |
| | | | THF/DMF | | | | | | |
| | | | 220/70 v/v | | | | | | |
| N1 | B16[α- | OEt | from N1—H, | 72 | AcOEt | 0.33 | ESI: (M + H)⁺ = 628/ | 1739, | 189-191 |
| | CO₂Et] | | HO₂C—B16[α- | | | | 630 | 1653 | |
| | | | CO₂Et]-OEt, | | | | (Br) | (C═O) | |
| | | | TBTU and NEt₃ | | | | | | |
| | | | in THF/DMF | | | | | | |
| | | | 150/50 v/v | | | | | | |
| N1 | B20[α- | OEt | from N1—H, | 100 | D | 0.73 | M⁺ = 657 | 1736, | colourless |
| | CO₂Et] | | HO₂C—B20[α- | | | | | 1668, | viscous oil |
| | | | CO₂Et]-OEt, | | | | | 1649 | |
| | | | TBTU, HOBt and | | | | | (C═O) | |
| | | | DIEA in | | | | | | |
| | | | THF/H₂O 10/1 v/v | | | | | | |
| N1 | B22[α- | OEt | from N1—H, | 88 | D | 0.78 | | 1734, | colourless |
| | CO₂Et] | | HO₂C—B22[α- | | | | | 1668 | crystals |
| | | | CO₂Et]-OEt, | | | | | (C═O) | |
| | | | TBTU, HOBt and | | | | | | |
| | | | DIEA in | | | | | | |
| | | | THF/H₂O 10/1 v/v | | | | | | |
| N1 | B25[α- | OEt | from N1—H, | 83 | AcOEt | 0.55 | M⁺ = 667/ | 1728, | colourless |
| | CO₂Et] | | HO₂C—B25[α- | | | | 669/671/ | 1664, | viscous oil |
| | | | CO₂Et]-OEt, | | | | 673 (BrCl₂) | 1645 | |
| | | | TBTU, HOBt and | | | | | (C═O) | |
| | | | DIEA in | | | | | | |
| | | | THF/H₂O 10/1 v/v | | | | | | |
| N1 | B27[α- | OEt | from N1—H, | 88 | AcOEt | 0.56 | | 1732, | colourless |
| | CO₂Et] | | HO₂C—B27[α- | | | | | 1668 | crystals |
| | | | CO₂Et]-OEt, | | | | | (C═O) | |
| | | | TBTU and NEt₃ | | | | | | |
| | | | in THF/DMF | | | | | | |
| | | | 250/10 v/v | | | | | | |
| N1 | B29[α- | OEt | from N1—H, | 87 | D | 0.79 | | 1753, | |
| | CO₂Et] | | HO₂C—B29[α- | | | | | 1728, | |
| | | | CO₂Et]-OEt, | | | | | 1660 | |
| | | | TBTU, HOBt and | | | | | (C═ 0) | |
| | | | DIEA in | | | | | | |
| | | | THF/H2O 10/1 v/v | | | | | | |
| N1 | B21[α- | OEt | from N1—H, | 75 | D | 0.74 | | | colourless |
| | CO₂Et] | | HO₂C—B21[α- | | | | | | crystals |
| | | | CO₂Et]-OEt, | | | | | | |
| | | | TBTU, HOBt and | | | | | | |
| | | | DIEA in | | | | | | |
| | | | THF/H2O 10/1 v/v | | | | | | |
| N1 | B30[α- | OEt | from N1—H, | 93 | F | 0.90 | ESI: (M + H)⁺ = 678/ | | colourless |
| | CO₂Et] | | HO₂C—B30[α- | | | | 680/682 | | crystals |
| | | | CO₂Et]-OEt, | | | | (Br₂) | | |
| | | | TBTU, HOBt and | | | | | | |
| | | | DIEA in | | | | | | |
| | | | THF/H2O 10/1 v/v | | | | | | |
| N1 | B23 | OMe | from N1—H, | 100 | | | | | |
| | | | HO₂C—B23—OMe, | | | | | | |
| | | | TBTU, HOBt and | | | | | | |
| | | | NEt₃ in THF | | | | | | |
| N1 | B24[α- | OEt | from N1—H, | 95 | D | 0.82 | | | colourless |
| | CO₂Et] | | HO₂C—B24[α- | | | | | | crystals |
| | | | CO₂Et]-OEt, | | | | | | |
| | | | TBTU, HOBt and | | | | | | |
| | | | DIEA in | | | | | | |
| | | | THF/H2O 10/1 v/v | | | | | | |
| N6 | B21[α- | OEt | from N6—H, | 86 | AcOEt | 0.9 | M⁺ = 727/ | 1734 | colourless |
| | CO₂Et] | | HO₂C—B21[α- | | | | 729/731 | (C═O) | viscous oil |
| | | | CO₂Et]-OEt, | | | | (Br₂) | | |
| | | | TBTU, HOBt and | | | | | | |
| | | | NEt₃ in | | | | | | |
| | | | THF/DMF 5/1 v/v | | | | | | |

### EXAMPLE A4

(R,S)-3,4-dibromo-phenylalanine ethyl ester

The mixture of 37.40 g (0.140 mol) N-(diphenylmethylene)-glycine ethyl ester, 55.0 g (0.167 mol) (3,4-dibromophenyl)-methylbromide, 6.40 g (0.020 mol) tetrabutylammonium bromide, 57.80 g (0.35 mol) potassium carbonate sesquihydrate and 1000 ml acetonitrile was refluxed for 15 hours. The solid was filtered off, the mother liquor was concentrated by evaporation in vacuo. The residue was taken up in 400 ml diethyl ether and after the addition of 200 ml semiconcentrated hydrochloric acid stirred for 1 hour at room temperature. The organic phase was separated off, the aqueous phase was washed twice more with 50 ml diethyl ether, then neutralised with solid sodium hydrogen carbonate while being cooled externally with ice and exhaustively extracted with ethyl acetate. The combined ethyl acetate extracts were dried over magnesium sulphate, filtered and evaporated down in vacuo. The product was obtained as a light brown oil.

Yield: 33.0 g (67% of theory). R_{f} 0.65 (EI D).

IR (KBr): 1734 (C═O) cm⁻¹

The following compounds of general formula N-B-C were prepared analogously:

| N | B | C | Remarks | % yield | EI | R_{f} | MS | IR [cm⁻¹] | mp. [° C.] |
|---|---|---|---|---|---|---|---|---|---|
| H | B6 | OEt | from Ph₂C═NCH₂CO₂Et | 60 | | | ESI: (M + H)⁺ = 300/ | 1738 | colourless |
| | | | and 3-Br-4,5-Me₂—C₆H₂— | | | | 302 | (C═O) | oil |
| | | | CH₂Br | | | | (Br) | | |
| H | B7 | OEt | from Ph₂C═NCH₂CO₂Et | 60 | P | 0.75 | | 1738 | colourless |
| | | | and 3,5-Br₂-4-Me—C₆H₂— | | | | | (C═O) | oil |
| | | | CH₂Br | | | | | | |
| H | B4 | OEt | from Ph₂C═NCH₂CO₂Et | 70 | B | 0.73 | ESI: (M + H)⁺ = 276/ | 1728 | colourless |
| | | | and 3,5-Cl₂-4-Me—C₆H₂— | | | | 278/280 | (C═O) | crystals, |
| | | | CH₂Br | | | | (Cl₂) | | mp. 44-46 |
| H | B8 | OEt | from Ph₂C═NCH₂CO₂Et | 83 | O | 0.46 | | 1736 | |
| | | | and 3-Cl-4-Me—C₆H₃— | | | | | (C═O) | |
| | | | CH₂Cl | | | | | | |

### EXAMPLE A5

(R,S)-3,4-difluorophenylalanine methyl ester hydrochloride

4.0 ml saturated methanolic hydrogen chloride solution were added to a suspension of 0.5 g (2.485 mmol) of 3,4-difluorophenylalanine in 10 ml of methanol and the mixture was stirred for 4 hours at room temperature. It was then evaporated down in vacuo, another 10 ml of methanol were added to the residue and the solvent was distilled off again in vacuo. 0.6 g (96% of theory) of colourless crystals were obtained, R_{f} 0.7 (EI dichloromethane).

ESI-MS: (M+H)⁺=216

### EXAMPLE A6

β,β-bis-(ethoxycarbonyl)-3,5-dibromo-4-fluoro-benzene-butanoic acid

70 ml trifluoroacetic acid were added dropwise to an ice-cooled solution of 13.1 g (0.037 mol) 1,1-dimethylethyl β,β-bis-(ethoxycarbonyl)-3,5-dibromo-4-fluoro-benzenebutanoate in 450 ml dichloromethane, the cooling was removed, the mixture was stirred overnight at ambient temperature and then evaporated down in vacuo. The residue was dried twice by coevaporation with petroleum ether, triturated with petroleum ether, suction filtered and dried in vacuo. 9.3 g (79% of theory) of colourless crystals were obtained.

IR (KBr): 1707 (C═O) cm⁻¹

ESI-MS: (M−H)-=481/483/485 (Br₂)

The following compounds of general formula N-B-C were prepared analogously:

| N | B | C | Remarks | % yield | EI | R_{f} | MS | IR [cm⁻¹] | mp. [° C.] |
|---|---|---|---|---|---|---|---|---|---|
| HO | B15[α- | OEt | from (H₃C)₃CO₂C— | 81 | V | 0.1 | | 1709 (C═O) | |
| | CO2Et] | | B15[α-CO₂Et]-OEt | | | | | | |
| | | | and TFA in CH₂Cl₂ | | | | | | |
| HO | B16[α- | OEt | from (H₃C)₃CO₂C— | 100 | | | | 1738 (C═O) | colourless |
| | CO2Et] | | B16[α-CO₂Et]-OEt | | | | | | viscous oil |
| | | | and TFA in CH₂Cl₂ | | | | | | |
| HO | B20[α- | OEt | from (H₃C)₃CO₂C— | 77 | V | 0.24 | | 3321 (OH); | colourless |
| | CO2Et] | | B20[α-CO₂Et]-OEt | | | | | 1714 | crystals |
| | | | and TFA in CH₂Cl₂ | | | | | (C═O); | |
| | | | | | | | | 1161, 1124 | |
| | | | | | | | | (CF₃) | |
| HO | B22[α- | OEt | from (H₃C)₃CO₂C— | 69 | W | 0.21 | | 1736 (C═O) | colourless |
| | CO2Et] | | B22[α-CO₂Et]-OEt | | | | | | crystals |
| | | | and TFA in CH₂Cl₂ | | | | | | |
| HO | B25[α- | OEt | from (H₃C)₃CO₂C— | 72 | | | | 1730, 1711 | colourless |
| | CO2Et] | | B25[α-CO₂Et]-OEt | | | | | (C═O) | viscous oil |
| | | | and TFA in CH₂Cl₂ | | | | | | |
| HO | B27[α- | OEt | from (H₃C)₃CO₂C— | 93 | | | | 1736 (C═O) | |
| | CO2Et] | | B27[α-CO₂Et]-OEt | | | | | | |
| | | | and TFA in CH₂Cl₂ | | | | | | |
| HO | B24[α- | OEt | from (H₃C)₃CO₂C— | 68 | X | 0.28 | | 1709 (C═O) | colourless |
| | CO2Et] | | B24[α-CO₂Et]-OEt | | | | | | crystals |
| | | | and TFA in CH₂Cl₂ | | | | | | |
| HO | B19[α- | OEt | from (H₃C)₃CO₂C— | 46 | | | | | |
| | CO2Et] | | B19[α-CO₂Et]-OEt | | | | | | |
| | | | and TFA in CH₂Cl₂ | | | | | | |
| HO | B30[α- | OEt | from (H₃C)₃CO₂C— | 81 | | | ESI: (M − H)⁻ = 463/ | | colourless |
| | CO2Et] | | B30[α-CO₂Et]-OEt | | | | 465/467 | | crystals |
| | | | and TFA in CH₂Cl₂ | | | | (Br₂) | | |
| HO | B24[α- | OEt | from (H₃C)₃CO₂C— | 54 | | | ESI: (M − H)⁻ = 375/ | | colourless |
| | CO2Et] | | B24[α-CO₂Et]-OEt | | | | 377/379 | | crystals |
| | | | and TFA in CH₂Cl₂ | | | | (Cl₂) | | |

### EXAMPLE A7

1,1-dimethylethyl 3,5-dibromo-4-fluoro-β,β-bis-(ethoxycarbonyl)-benzenebutanoate

0.64 g (0.0266 mol) 95% sodium hydride were added to the solution of 6.69 g (0.024 mol) diethyl [(1,1-dimethylethoxy-carbonyl)methyl]-malonate in 170 ml anhydrous tetrahydrofuran while cooling externally with ice water. After one hour's stirring a solution of 8.35 g (0.024 mol) 3,5-dibromo-4-fluorobenzylbromide in 30 ml of tetrahydrofuran was added dropwise thereto while maintaining a reaction temperature of 0 to +5° C. and the mixture was then allowed to come up to room temperature within 14 hours. The reaction mixture was freed from solvent in vacuo, the residue was combined with 200 ml 10% citric acid and exhaustively extracted with tert.-butylmethylether. After working up in the usual way the combined extracts yielded 13.1 g (100% of theory) of a colourless oil, R_{f}=0.14

(EI Y), which was used in the next step without any purification.

IR (KBr): 1732 (C═O) cm⁻¹

ESI-MS: (M+Na)⁺=561/563/565 (Br₂)

The following compounds of general formula N-B-C were prepared analogously:

| | | | | % | | | | IR | |
|---|---|---|---|---|---|---|---|---|---|
| N | B | C | Remarks | yield | EI | R_{f} | MS | [cm⁻¹] | mp. [° C.] |
| Me₃CO | B15[α- | OEt | from (H₃C)₃COCO— | 100 | V | 0.6 | | | colourless |
| | CO2Et] | | CH₂C(CO₂Et)₂, | | | | | | oil |
| | | | 3,4,5-Me₃— | | | | | | |
| | | | C₆H₂CH₂Br and | | | | | | |
| | | | NaH in THF | | | | | | |
| Me₃CO | B16[α- | OEt | from (H₃C)₃COCO— | 67 | CH₂Cl₂ | 0.71 | | 1736 | colourless |
| | CO2Et] | | CH₂C(CO₂Et)₂, | | | | | (C═O) | oil |
| | | | 3Br-4,5-Me₂— | | | | | | |
| | | | C₆H₂CH₂Br and | | | | | | |
| | | | NaH in THF | | | | | | |
| Me₃CO | B20[α- | OEt | from (H₃C)₃COCO— | 100 | V | 0.72 | no M⁺; | 1736 | |
| | CO2Et] | | CH₂C(CO₂Et)₂, 2,4- | | | | (M − C₄H₈)⁺ = 444 | (C═O) | |
| | | | (CF₃)₂—C₆H₂CH₂Br | | | | | | |
| | | | and NaH in THF | | | | | | |
| Me₃CO | B22[α- | OEt | from (H₃C)₃COCO— | 91 | W | 0.78 | | 1734 | colourless |
| | CO2Et] | | CH₂C(CO₂Et)₂, | | | | | (C═O) | oil |
| | | | 3,4,5Br₃— | | | | | | |
| | | | C₆H₂CH₂Br and | | | | | | |
| | | | NaH in THF | | | | | | |
| Me₃CO | B25[α- | OEt | from (H₃C)₃COCO— | 100 | Y | 0.75 | | | colourless |
| | CO2Et] | | CH₂C(CO₂Et)₂, 4- | | | | | | viscous oil |
| | | | Br-3,5Cl₂— | | | | | | |
| | | | C₆H₂CH₂Br and | | | | | | |
| | | | NaH in THF | | | | | | |
| Me₃CO | B27[α- | OEt | from (H₃C)₃COCO— | 58 | Y | 0.31 | M⁺ = 406 | 1734 | |
| | CO2Et] | | CH₂C(CO₂Et)₂, 3,4 | | | | | (C═O) | |
| | | | (CH₂)₂O— | | | | | | |
| | | | C₆H₃CH₂Br and | | | | | | |
| | | | NaH in THF | | | | | | |
| Me₃CO | B29[α- | OEt | from (H₃C)₃COCO— | 89 | X | 0.49 | | 1736 | colourless |
| | CO2Et] | | CH₂C(CO₂Et)₂, | | | | | (C═O) | oil |
| | | | 2,3Cl₂—C₆H₃CH₂Cl | | | | | | |
| | | | and NaH in THF | | | | | | |
| Me₃CO | B19[α- | OEt | from (H₃C)₃COCO— | 88 | | | | | colourless |
| | CO2Et] | | CH₂C(CO₂Et)₂, | | | | | | oil |
| | | | 4NH₄-3,5Cl₂— | | | | | | |
| | | | C₆H₂CH₂Br and | | | | | | |
| | | | NaH in THF | | | | | | |

### EXAMPLE A8

3,4-dimethoxy-β-(methoxycarbonyl)-benzenebutanoic acid

The solution of 58.0 g (0.205 mol) 4-[(3,4-dimethoxyphenyl]-3-(methoxycarbonyl)-3-butenoic acid in 500 ml of methanol was hydrogenated at 5 bar hydrogen in the presence of 3.0 g 10% platinum/activated charcoal until the uptake of hydrogen had ended. After working up in the usual way 26.0 g (46% of theory) of colourless crystals were obtained, mp=104-107° C.

The following compound of general formula N-B-C was obtained analogously:

| | | | | % | | | mp. |
|---|---|---|---|---|---|---|---|
| N | B | C | Remarks | yield | EI | R_{f} | [° C.] |
| HO | B26 | OMe | from 4-(2-naphthyl)-3- | | X | 0.85 | |
| | | | (methoxycarbonyl)-3- | | | | |
| | | | butenoic acid, | | | | |
| | | | H₂ and Pd—C in MeOH | | | | |

### EXAMPLE A9

4-[(3,4-dimethoxy-phenyl]-3-(methoxycarbonyl)-3-butenoic acid

26.6 ml (0.2 mol) dimethyl succinate were added to a freshly prepared solution of 4.6 g (0.2 mol) sodium in 250 ml anhydrous methanol and after one hour's stirring at room temperature the solution of 33.3 g (0.2 mol) 3,4-dimethoxybenzaldehyde in 100 ml anhydrous methanol was added dropwise. Then the mixture was refluxed for 6 hours, the methanol was eliminated in vacuo and the bottom remaining was maintained at a reaction temperature of 80° C. for 30 minutes. The viscous slurry obtained was taken up in 500 ml of water, acidified with 20% aqueous citric acid solution and the resulting mixture was exhaustively extracted with ethyl acetate. The combined ethyl acetate extracts were in turn extracted five times with 5% aqueous ammonia solution. The ammoniacal extracts were carefully acidified with 20% aqueous citric acid solution and then exhaustively extracted with ethyl acetate. These extracts were washed with water, dried over sodium sulphate and freed from the solvent in vacuo. The crude product (quantitative yield) was further reacted without purification.

The following compounds of general formula N-B-C were obtained analogously:

| | | | | | | |
|---|---|---|---|---|---|---|
| | | | | % | | |
| N | B | C | Remarks | yield | EI | R_{f} |

| | | | | |
|---|---|---|---|---|
| 4-(2-naphthyl)-3- | from 2-naphthaldehyde, | 65 | X | 0.8 |
| (methoxycarbonyl)- | dimethyl succinate and | | | |
| 3-butenoic acid | NaOMe in MeOH | | | |

### EXAMPLE A10

Methyl [1,4′]bipiperidinyl-4-acetate

The solution of 0.669 g (2.024 mmol) of methyl 1′-phenylmethyl-[1,4′]bipiperidinyl-4-acetate in 20 ml of methanol was hydrogenated at a pressure of 5 bar after the addition of 100 mg of 10% palladium on charcoal until the uptake of hydrogen had ended. The catalyst was filtered off, the filtrate was freed from solvent, the residue was taken up in 20 ml THF, the solution obtained was filtered and evaporated down again. The residue was used without further purification. Colourless oil.

Yield: 490 mg (100% of theory).

ESI-MS: (M+H)⁺=241 (M+Na)⁺=253

The following compounds of general formula N-B-C were prepared analogously:

| N | B | C | Remarks | % yield | EI | R_{f} | MS | mp. [° C.] |
|---|---|---|---|---|---|---|---|---|
| H | — | C5 | from PhCH₂—C5, H₂ | 100 | G | 0.22 | ESI: (M + H)⁺ = 241; | colourless |
| | | | and Pd/C in MeOH | | | | (M + Na)⁺ = 253 | oil |
| H | — | C12 | from PhCH₂—C12, H₂ | 98 | D | 0.17 | ESI: (M + H)⁺ = 284 | colourless |
| | | | and Pd/C in EtOH | | | | | crystals |
| H | — | C9 | from PhCH₂—C9, H₂ | 78 | O | 0.1 | | colourless |
| | | | and Pd/C in EtOH | | | | | oil |
| H | — | C3 | from PhCH₂—C3, H₂ | 99 | | | ESI: (M + H)⁺ = 284; | colourless |
| | | | and Pd/C in MeOH | | | | (M + Na)⁺ = 306 | oil |
| H | — | C1 | from PhCH₂—C1, H₂ | 97 | M | 0.38 | ESI: (M + H)⁺ = 256 | |
| | | | and Pd/C in EtOH | | | | | |
| H | — | C14 | from PhCH₂—C14, H₂ | 79 | G | 0.14 | ESI: (M + H)⁺ = 213 | colourless |
| | | | and Pd/C in MeOH | | | | | crystals |
| H | — | C16 | from PhCH₂—C16, H₂ | 67 | G | 0.16 | ESI: (M + H)⁺ = 213 | colourless |
| | | | and Pd/C in MeOH | | | | | crystals |
| H | — | C19 | from PhCH₂—C19, H₂ | 100 | G | 0.20 | ESI: (M + H)⁺ = 227 | colourless |
| | | | and Pd/C in MeOH | | | | | oil |
| H | — | C22 | from PhCH₂—C22, H₂ | 100 | C | 0.06 | ESI: (M + H)⁺ = 227 | colourless |
| | | | and Pd/C in MeOH | | | | | crystals |
| H | — | C26 | from PhCH₂—C26, H₂ | 100 | | | | colourless |
| | | | and Pd/C in MeOH | | | | | crystals |
| H | — | C28 | from methyl 4-[(1- | 70 | S | 0.4 | | colourless |
| | | | phenylmethyl)-1,2,3,6- | | | | | crystals |
| | | | tetrahydro-4- | | | | | |
| | | | pyrididinyl]-benzoate, | | | | | |
| | | | H₂ and Pd/C in MeOH | | | | | |
| H | — | C18 | acetate, from PhCH₂— | 88 | G | 0.20 | ESI: (M + H)⁺ = 227 | colourless |
| | | | C18, H₂ and Pd/C in | | | | | viscous oil |
| | | | MeOH | | | | | |
| H | — | C7 | from PhCH₂—C7, H₂ | 92 | O | 0.15 | ESI: (M + H)⁺ = 241; | colourless |
| | | | and Pd/C in EtOH | | | | (M + Na)⁺ = 263 | oil |
| H | — | C50 | from PhCH₂—C50, H₂ | 100 | KK | 0.21 | ESI: (M + H)⁺ = 256 | colourless |
| | | | and Pd(OH)₂ | | | | | viscous oil |
| | | | (Pearlman's catalyst) | | | | | |
| | | | in EtOH | | | | | |
| ethyl 4- | | | from ethyl 1- | 99 | | | | colourless |
| methyl-2- | | | (phenylmethyl)-4- | | | | | oil |
| piperazine- | | | methyl-2- | | | | | |
| carboxylate | | | piperazinecarboxylate, | | | | | |
| | | | H₂ and Pd(OH)₂ | | | | | |
| | | | (Pearlman's catalyst) | | | | | |
| | | | in EtOH | | | | | |
| H | — | C46 | from PhCH₂—C46, H₂ | 100 | DD | 0.24 | ESI: (M + H)⁺ = 256 | colourless |
| | | | and Pd(OH)₂ | | | | | viscous oil |
| | | | (Pearlman's catalyst) | | | | | |
| | | | in EtOH | | | | | |
| H | — | C45 | from PhCH₂—C45, H₂ | 100 | LL | 0.1 | ESI: (M + H)⁺ = 256 | colourless |
| | | | and Pd(OH)₂ | | | | | oil |
| | | | (Pearlman's catalyst) | | | | | |
| | | | in EtOH | | | | | |

| | | | | | |
|---|---|---|---|---|---|
| ethyl 2- | from ethyl 1,4-bis- | 100 | MM | 0.2 | ESI: (M + H)⁺ = 159 |
| piperazine- | (phenylmethyl)-2- | | | | |
| carboxylate | piperazinecarboxylate, | | | | |
| | H₂ and 10% Pd/C in | | | | |
| | EtOH | | | | |

### EXAMPLE A11

Methyl 1′-(phenylmethyl)-[1,4′]bipiperidinyl-4-acetate

4.0 ml glacial acetic acid and 20 g of molecular sieve 3 Å were added to a mixture of 4.549 ml (24.54 mmol) of 1-(phenylmethyl)-4-piperidinene, 4.753 g (24.54 mmol) of methyl 4-piperidineacetate hydrochloride and 40 ml of THF, the mixture was stirred for 2 hours at room temperature, cooled to 0° C. and while this temperature was maintained a total of 6.358 g (30.0 mmol) of sodium triacetoxyborohydride were added in small batches within 8 hours. Then the resulting mixture was stirred for another 16 hours at room temperature. The mixture was made alkaline with sodium hydrogen carbonate, extracted exhaustively with ethyl acetate, the combined extracts were dried over sodium sulphate and the evaporation residue was chromatographed on silica gel using first 30/1 dichloromethane/methanol, then 20/1, and finally 10/1 as eluants. Working up the appropriate fractions yielded 1.804 g (22% of theory) of a readily mobile oil which set overnight into colourless crystals. R_{f}=0.56 (EI B).

ESI-MS: (M+H)⁺=331.

The following compounds of general formula N-B-C were prepared analogously:

| N | B | C | Remarks | % yield | EI | R_{f} | MS | mp. [° C.] |
|---|---|---|---|---|---|---|---|---|
| PhCH₂ | — | C7 + C9 | from 1-(phenylmethyl)- | cis: 14.7 + trans: | AA | cis: | cis: ESl: | colourless |
| | | | piperazine, ethyl 4- | 13.8 + cis/ | | 0.40; | (M + H)⁺ = 331; | liquids |
| | | | oxocyclohexane- | trans: | | trans: | (M + Na)⁺ = 353; | |
| | | | carboxylate and | 5.8 | | 0.30 | trans: | |
| | | | Na(CN)BH₃/AcOH in | | | | ESI: (M + H)⁺ = 331 | |
| | | | MeOH at pH 5-6; | | | | | |
| | | | separation of the two | | | | | |
| | | | diastereomers on silica | | | | | |
| | | | gel, El dichloromethane/ | | | | | |
| | | | MeOH 30/1 v/v | | | | | |
| PhCH₂ | — | C3 | from 1-(phenylmethyl)- | 58 | O | 0.67 | ESI: (M + H)⁺ = 374; | colourless |
| | | | piperazine, 1,1- | | | | (M + Na)⁺ = 396 | crystals |
| | | | dimethylethyl 4-oxo-1- | | | | | |
| | | | piperidineacetate and | | | | | |
| | | | Na(CN)BH₃/AcOH in | | | | | |
| | | | MeOH at pH 5-6 | | | | | |
| 4-[1-(phenylmethyl)-4- | | | from 1-(phenylmethyl)-4- | 100 | D | 0.60 | ESI: (M + H)⁺ = 360; | colourless |
| piperidinyl]-1-(1,1- | | | piperidinene, 1-(1,1- | | | | (M + Na)⁺ = 382; | oil |
| dimethylethoxycarbonyl)- | | | dimethylethoxycarbonyl)- | | | | (2M + Na)⁺ = 741 | |
| piperazine | | | piperazine and | | | | | |
| | | | NaBH(OAc)₃/AcOH in | | | | | |
| | | | THF | | | | | |
| PhCH₂ | — | C14 | from 1-(phenylmethyl)-4- | 51 | G | 0.50 | ESI: (M + H)⁺ = 303 | colourless |
| | | | piperidinene, L-proline | | | | | oil |
| | | | methyl ester | | | | | |
| | | | hydrochloride and | | | | | |
| | | | NaBH(OAc)₃/AcOH in | | | | | |
| | | | THF | | | | | |
| PhCH₂ | — | C16 | from 1-(phenylmethyl)-4- | 54 | G | 0.50 | ESI: (M + H)⁺ = 303; | colourless |
| | | | piperidinene, D-proline | | | | (M + Na)⁺ = 325 | oil |
| | | | methyl ester | | | | | |
| | | | hydrochloride and | | | | | |
| | | | NaBH(OAc)₃/AcOH in | | | | | |
| | | | THF | | | | | |
| PhCH₂ | — | C19 | from 1-(phenylmethyl)-4- | 51 | G | 0.40 | ESI: (M + H)⁺ = 317; | colourless |
| | | | piperidinene, L- | | | | (M + Na)⁺ = 339 | oil |
| | | | homoproline methylester | | | | | |
| | | | hydrochloride [Bachem] | | | | | |
| | | | and NaBH(OAc)₃ in | | | | | |
| | | | CH₂Cl₂ | | | | | |
| PhCH₂ | — | C18 | from 1-(phenylmethyl)-4- | 57 | G | 0.40 | ESI: (M + H)⁺ = 317 | colourless |
| | | | piperidinene, D- | | | | | viscous |
| | | | homoproline methylester | | | | | oil |
| | | | hydrochloride [Bachem] | | | | | |
| | | | and NaBH(OAc)₃ in | | | | | |
| | | | CH₂Cl₂ | | | | | |
| PhCH₂ | — | C50 | from 1-(phenylmethyl)-4- | 22 | DD | 0.84 | ESI: (M + H)⁺ = 346 | |
| | | | piperidinene, ethyl 4- | | | | | |
| | | | methyl-2- | | | | | |
| | | | piperazinecarboxylate | | | | | |
| | | | and NaBH(OAc)₃ in THF | | | | | |
| PhCH₂ | — | C46 | from 1-methyl-4- | 100 | C | 0.53 | ESI: (M + H)⁺ = 346 | colourless |
| | | | piperidinene, ethyl bis- | | | | | oil |
| | | | (trifluoroacetate) 1- | | | | | |
| | | | (phenylmethyl)-2- | | | | | |
| | | | piperazinecarboxylate- | | | | | |
| | | | and NaBH(OAc)₃ in THF | | | | | |
| PhCH₃ | — | C45 | from 1-(phenylmethyl)-4- | 100 | C | 0.41 | M⁺ = 345 | colourless |
| | | | piperidinene, ethyl bis- | | | | | oil |
| | | | (trifluoroacetate) 1- | | | | | |
| | | | methyl-2- | | | | | |
| | | | piperazinecarboxylate | | | | | |
| | | | and NaBH(OAc)₃ in THF | | | | | |
| Boc | — | C44 | from 1-methyl-4- | 57 | C | 0.46 | ESI: (M + H)⁺ = 356 | colourless |
| | | | piperidinene, ethyl -bis- | | | | | viscous |
| | | | (trifluoroacetate) 4-(1,1- | | | | | oil |
| | | | dimethylethoxycarbonyl)- | | | | | |
| | | | 2-piperazinecarboxylate | | | | | |
| | | | and NaBH(OAc)₃ in THF | | | | | |

### EXAMPLE A12

Ethyl 4-[1-(phenylmethyl)-4-piperidinyl]-1-piperazineacetate

3.5 ml (19.892 mmol) of DIEA were added to a suspension of 2.0 g (3.325 mmol) of 1-(phenylmethyl)-4-(1-piperazinyl)-piperidine-tris-(trifluoroacetate) in 50 ml dichloromethane and the mixture was stirred for 10 minutes at room temperature. Then 0.38 ml (3.365 mmol) of ethyl bromoacetate were added and the mixture was stirred overnight at room temperature. The reaction mixture was extracted four times with 50 ml of water, dried over sodium sulphate and concentrated by evaporation. 0.70 g (61% of theory) of the desired product were obtained, R_{f} 0.63 (EI D) and

ESI-MS: (M+H)⁺=346.

The following compound of general formula N-B-C was obtained analogously:

| | | | | % | | | | |
|---|---|---|---|---|---|---|---|---|
| N | B | C | Remarks | yield | EI | R_{f} | MS | mp. [° C.] |
| PhCH₂ | — | C12 | from 1-(phenylmethyl)-4- | 65 | D | 0.51 | ESI: (M + H)⁺ = 374; | colourless |
| | | | (1-piperazinyl)- | | | | (M + Na)⁺ = 396 | crystals |
| | | | piperidine-tris- | | | | | |
| | | | (trifluoroacetate), 1,1- | | | | | |
| | | | dimethylethyl | | | | | |
| | | | bromoacetate and | | | | | |
| | | | K₂CO₃ in CH₃CN | | | | | |

### EXAMPLE A13

1-(phenylmethyl)-4-(1-piperazinyl)-piperidine-tris-(trifluoroacetate)

The mixture of 77.6 g (0.216 mol) 4-[1-(phenylmethyl)-4-piperidinyl]-1-(1,1-dimethylethoxycarbonyl)-piperazine, 150 ml (1.941 mol) trifluoroacetic acid and 450 ml dichloromethane was refluxed for 1 hour and then stirred for 2 hours at room temperature. The solvent was distilled off, the residue triturated with diethyl ether, suction filtered and dried in the air. 119.0 g (92% of theory) of colourless crystals were obtained, R_{f} 0.20 (EI D) and

ESI-MS: (M+H)⁺=260

The following compounds of general formula N-B-C were prepared analogously:

| | | | | % | | | | |
|---|---|---|---|---|---|---|---|---|
| N | B | C | Remarks | yield | EI | R_{f} | MS | mp. [° C.] |
| H | — | C29 | from ethyl 4-[[1-(1,1- | 89 | BB | 0.70 | | colourless |
| | | | dimethylethoxycarbonyl)- | | | | | crystals |
| | | | 4-piperidinyl]methyl]- | | | | | |
| | | | benzoate and TFA in | | | | | |
| | | | CH₂Cl₂ | | | | | |
| H | — | C44 | from ethyl 4-(1,1- | 100 | DD | 0.11 | M⁺ = 255 | colourless |
| | | | dimethylethoxycarbonyl)- | | | | | viscous oil |
| | | | 1-(1-methyl-4-piperidinyl)- | | | | | |
| | | | 2-piperazinecarboxylate | | | | | |
| | | | and TFA in CH₂Cl₂ | | | | | |

| | | | | | | |
|---|---|---|---|---|---|---|
| ethyl-bis- | from ethyl 4-(1,1- | 100 | AcOEt | 0.00 | ESI: | colourless |
| (trifluoroacetate) | dimethylethoxycarbonyl)- | | | | (M + H)⁺ = 249 | oil |
| 1-(phenylmethyl)- | 1-(phenylmethyl)-2- | | | | | |
| 2-piperazine- | piperazinecarboxylate | | | | | |
| carboxylate | and TFA in CH₂Cl₂ | | | | | |
| ethyl-bis- | from ethyl 4-(1,1- | 100 | DD | 0.16 | ESI: | colourless |
| (trifluoroacetate)1- | dimethylethoxycarbonyl)- | | | | (M + H)⁺ = 173 | viscous oil |
| methyl-2- | 1-methyl-2- | | | | | |
| piperazine- | piperazinecarboxylate | | | | | |
| carboxylate | and TFA in CH₂Cl₂ | | | | | |

### EXAMPLE A14

methyl 1′-(phenylmethyl)-[1,4′]bipiperidinyl-4′-carboxylate

1.124 g (3.5 mmol) of TBTU and 1.0 ml (7.175 mmol) of triethylamine were added to the solution of 1.0 g (3.307 mmol) of 1′-(phenylmethyl)-[1,4′]bipiperidinyl-4′-carboxylic acid in 30 ml DMF, the mixture was stirred for 20 minutes at room temperature, then 20 ml of methanol were added and the mixture was stirred for a further 3 hours at ambient temperature. The mixture was concentrated by evaporation, the residue was taken up in 50 ml of ethyl acetate and filtered. The filtrate was evaporated down, the residue purified by column chromatography on silica gel, initially using ethyl acetate, then ethyl acetate mixed with up to 5% methanol/conc. ammonia (9/1 v/v) as eluant. 0.231 g (22% of theory) of colourless crystals were obtained, mp. 84.7° C. and R_{f} 0.73 (EI F).

ESI-MS: (M+H)⁺=317

### EXAMPLE A15

Methyl 3-(4-piperidinyl)-benzoate-hydrochloride

The mixture of 500 mg (2.069 mmol) of 3-(4-piperidinyl)-benzoic acid-hydrochloride and 10 ml saturated methanolic hydrogen chloride solution was stirred overnight at room temperature. The reaction mixture was concentrated by evaporation in vacuo, the residue was stirred with 3 ml isopropanol, suction filtered, washed with diethyl ether and dried at 60° C. in the circulating air dryer. 390 mg (74% of theory) of colourless crystals were obtained, R_{f} 0.34 (EI D).

IR (KBr): 1728 (C═O) cm⁻¹

ESI-MS: (M+H)⁺=220;

(M+Cl+HCl)=290/292/294 (Cl₂)

The following esters of general formula N-B-C were obtained analogously:

| | | | | % | | | | IR | mp. |
|---|---|---|---|---|---|---|---|---|---|
| N | B | C | Remarks | yield | EI | R_{f} | MS | [cm⁻¹] | [° C.] |
| H | — | C31 | dihydrochloride; from | 76 | D | 0.58 | ESI: (M + H)⁺ = 289; | 1722 | colourless |
| | | | H-C38 [BAYER], | | | | (M + Cl + HCl)⁻ = | (C═O) | crystals |
| | | | MeOH and HCl | | | | 359/361/363 | | |
| | | | | | | | (Cl₂) | | |
| PhCH₂ | — | C41 | from PhCH₂-C43, | 52 | D | 0.88 | ESI: (M + H)⁺ = 318; | | |
| | | | MeOH and HCl | | | | (M + Na)⁺ = 340; | | |
| | | | | | | | (2M + Na)⁺ = 657 | | |

| | | | | | | |
|---|---|---|---|---|---|---|
| methyl 2- | from 2- | 100 | D | 0.59 | ESI: (M + H)⁺ = 159; | |
| aminothiazole-5- | aminothiazole-5- | | | | (M − H)⁻ = 157 | |
| carboxylate | carboxylic acid, | | | | | |
| hydrochloride | MeOH and HCl | | | | | |
| methyl 4-[1- | from 4-[1- | 85 | | | ESI: (M + H)⁺ = 308 | 1707 |
| (phenylmethyl)- | (phenylmethyl)- | | | | | (C═O) |
| 1,2,3,6-tetrahydro-4- | 1,2,3,6-tetrahydro-4- | | | | | |
| pyridinyl]-benzoate | pyridinyl]-benzoic | | | | | |
| | acid, MeOH and HCl | | | | | |

### EXAMPLE A16

1′-(phenylmethyl)-[1,4′]bipiperidinyl-4′-carboxylic acid

A total of 5.0 g (17.642 mmol) of 1′-(phenylmethyl)-[1,4′]bipiperidinyl-4′-carbonitrile were added in small batches to 15 ml of conc. sulphuric acid. After the nitrile had dissolved, the mixture was stirred for a further 3 hours at room temperature, then ml of water were added and the mixture was refluxed for 15 hours. The cooled mixture was stirred into 50 ml ice water and adjusted to pH 7 with conc. ammonia. The precipitate was suction filtered, washed with a little water, stirred with 10 ml dichloromethane, suction filtered again, then dried in vacuo. 1.56 g (29% of theory) of colourless crystals were obtained, R_{f} 0.0 (EI DD).

ESI-MS: (M+H)=303

### EXAMPLE A17

Ethyl 3-(1-piperazinyl)-benzoate

30 ml of a saturated solution of hydrogen bromide in glacial acetic acid was added dropwise at room temperature to the solution of 18.5 g (0.055 mol) ethyl 3-[4-(phenylmethoxycarbonyl)-1-piperazinyl]-benzoate in 30 ml glacial acetic acid and stirred for a further 4 hours at room temperature. 300 ml diethyl ether were added to the mixture, the precipitate formed was then suction filtered, washed thoroughly with diethyl ether and dried in the air.

Yield 17.8 g (82% of theory). Colourless crystals, mp. 226° C. (Z) and R_{f} 0.24 (EI EE).

C₁₃H₁₈N₂O_{2*2} HBr (396.13) Calc.: C, 39.42H, 5.09N, 7.07 Br, 40.34 Found: 39.27 5.06 7.15 40.35

### EXAMPLE A18

Ethyl 3-[4-(phenylmethoxycarbonyl)-1-piperazinyl]-benzoate

At intervals of 16 hours 15.0 g (a total of 0.176 mol) of benzyl chlorocarbonate were added twice to the solution of 26.0 g (0.08 mol) ethyl 3-[4-(phenylmethyl)-1-piperazinyl]-benzoate in 260 ml dichloromethane and the mixture was stirred for a total of 32 hours at room temperature. The solvent was eliminated in vacuo, the residue purified by column chromatography on silica gel using dichloromethane as eluant. 18.8 g (70% of theory) of a colourless oil were obtained, R_{f} 0.67 (EI FF).

### EXAMPLE A19

Ethyl 3-[4-(phenylmethyl)-1-piperazinyl]-benzoate-hydriodide

The mixture of 53.6 g (0.2 mol) N,N-bis-(2-chlorethyl)-benzenemethanamine-hydrochloride, 40.2 g (0.2 mol) ethyl 3-aminobenzoate-hydrochloride, 30.0 g (0.2 mol) sodium iodide, 20.0 g sodium carbonate and 11 of n-propanol was refluxed for 2 hours. The mixture was cooled to 80° C., a further 15 g of sodium carbonate were added slowly and the mixture was refluxed for another 2 hours. After cooling to 80° C. the remaining sodium carbonate from a total amount of 53.0 g (0.5 mol) was added and again the mixture was refluxed for 2 hours. It was left to cool, the insoluble salts were filtered off and the filtrate was evaporated down in vacuo. The residue was taken up in 200 ml dichloromethane, the dichloromethane solution was washed twice with 50 ml 1 N hydrochloric acid, then concentrated by evaporation. After being recrystallised from ethanol the residue remaining yielded 43.0 g (48% of theory) of colourless crystals, mp. 180-182° C. and R_{f}=0.62 (EI GG).

### EXAMPLE A20

4-[1-(phenylmethyl)-1,2,3,6-tetrahydro-4-pyridinyl]-benzoic acid

25.0 ml (0.04 mol) of a 1.6-molar solution of n-butyl lithium in n-hexane were added dropwise to the solution of 13.13 g (0.040 mol) 4-(4-bromophenyl)-1-(phenylmethyl)-1,2,3,6-tetrahydropyridine in 190 ml of anhydrous THF under an argon atmosphere and while maintaining a reaction temperature of −70 to −60° C. After 30 minutes at −60° C. the mixture was poured, while stirring well, onto 500 g of finely crushed dry ice and the mixture was then left overnight to come up to room temperature. It was diluted with 300 ml diethyl ether and then extracted twice with 100 ml of water. While cooling externally, the combined aqueous extracts were adjusted to pH 7.5 with 2N hydrochloric acid. The precipitate formed was suction filtered, stirred with 50 ml hot methanol and after cooling suction filtered again. After drying in the desiccator 8.3 g (71% of theory) of colourless crystals were obtained, R_{f} 0.5 (EI HH).

ESI-MS: (M+H)⁺=294 (M−H)⁻=292

### EXAMPLE A21

4-(4-Bromophenyl)-1-(phenylmethyl)-4-piperidinel

62.5 ml (0.1 mol) of a 1.6 molar solution of n-butyl lithium in n-hexane were added dropwise to the solution of 23.591 g (0.10 mol) 1,4-dibromobenzene in 250 ml anhydrous THF while maintaining a reaction temperature of −60 to −50° C. The mixture was stirred for a further 20 minutes at the stated temperature before the solution of 18.926 g (0.10 mol) 1-(phenylmethyl)-4-piperidinene in 50 ml anhydrous THF was added dropwise. The mixture was allowed to warm up to room temperature, then stirred overnight at this temperature, the mixture was then added to ice water and exhaustively extracted with ethyl acetate. The combined ethyl acetate extracts were washed with water and saturated saline solution, dried over sodium sulphate and concentrated by evaporation in vacuo. The residue was recrystallised from diisopropylether. 23.1 g (67% of theory) of colourless crystals were obtained, R_{f} 0.4 (EI BB).

### EXAMPLE A22

Ethyl 4-[[1-(1,1-dimethylethoxycarbonyl)-4-piperidinyl]methyl]-benzoate The solution of 38.7 g (0.112 mol) 1-(1,1-dimethylethoxycarbonyl)-4-[4-(ethoxy-carbonyl)-phenylmethylene]-piperidine in 350 ml of ethyl acetate was hydrogenated at room temperature and under a pressure of 5 bar in the presence of 4.82 g 10% palladium on charcoal until the uptake of hydrogen had ended. Working up in the usual way yielded 35.8 g (92% of theory) of a colourless oil which was used without any further purification.

### EXAMPLE A23

1-(1,1-dimethylethoxycarbonyl)-4-[4-(ethoxycarbonyl)-phenylmethylene]-piperidine

85.0 ml (0.136 mol) of a 1.6 molar solution of n-butyl lithium in n-hexane was added dropwise to the solution of 19.2 ml (0.135 mol) diisopropylamine in 400 ml anhydrous THF using argon as protective gas and while maintaining a reaction temperature of −20 to −10° C. This temperature was maintained for another 20 minutes and then the solution of 39.35 g (0.131 mol) diethyl [4-(ethoxy-carbonyl)phenyl]-methanephosphonate in 100 ml THF was added dropwise. The mixture was stirred for a further 20 minutes at a temperature between −20 and −10° C., then the solution of 28.1 g (0.131 mol) 1-(1,1-dimethylethoxy-carbonyl)-4-piperidinene in 100 ml THF was added dropwise thereto and the mixture was left overnight to warm up to room temperature. The mixture was stirred into ice water, the resulting mixture was exhaustively extracted with ethyl acetate, the combined extracts were washed with saturated aqueous NaCl solution, dried over sodium sulphate and freed from solvent. The residue was purified by column chromatography on silica gel using petroleum ether/ethyl acetate 7/1 v/v as eluant. 38.7 g (86% of theory) of a colourless oil were obtained, which solidified in the presence of petroleum ether to form colourless crystals.

### EXAMPLE A24

Diethyl [4-(ethoxycarbonyl)phenyl]-methanephosphonate

55 ml (0.316 mol) triethyl phosphite were placed in a stirring apparatus and pre-heated to an internal temperature of 90° C. The suspension of 60.0 g (0.247 mol) ethyl 4-(bromomethyl)-benzoate in 100 ml dichloromethane was slowly added thereto in small batches, while the ethyl bromide formed and the evaporating dichloromethane were continuously distilled off. Once the quantity of ethyl bromide formed had significantly diminished, the reaction temperature was slowly increased to 140° C. and this temperature was maintained until the formation of ethyl bromide had ended (approx. 2 hours). The excess triethyl phosphite was eliminated in vacuo, the residue was suspended in a little ethyl acetate and purified by column chromatography on silica gel using ethyl acetate/petroleum ether (gradient 1/1→1/0 v/v) as eluant. After working up in the usual way 56.3 g (76% of theory) of the above title compound were obtained in the form of a colourless oil.

### EXAMPLE A25

Ethyl 4-[2-(4-piperidinyl)ethyl]-benzoate

The solution of 22.0 g (0.076 mol) ethyl 4-[2-(4-pyridinyl)vinyl]-benzoate hydrochloride in 800 ml of ethanol was hydrogenated in the presence of 2 g platinum(IV)-oxide at 3.8 bar hydrogen pressure for 8 hours. Catalyst and solvent were removed, the residue was taken up in 5% hydrochloric acid and extracted twice with 50 ml diethyl ether. The aqueous phase was made alkaline with sodium hydroxide and exhaustively extracted with ethyl acetate. The combined ethyl acetate extracts were washed with saturated saline solution, dried over sodium sulphate and concentrated by evaporation. The oily product obtained (17.0 g, 86% of theory) was used without further purification.

### EXAMPLE A26

Ethyl (E)-4-[2-(4-pyridinyl)vinyl]-benzoate hydrochloride

A solution of 9.1 g (85.0 mmol) of 4-pyridine-carboxaldehyde and 25.0 g (83.3 mmol) of diethyl [4-(ethoxycarbonyl)phenyl]-methanephosphonate in 150 ml THF was added dropwise to a suspension of 1.87 g (78 mmol) of sodium hydride in 150 ml THF while maintaining a reaction temperature of −10 to 0° C. The mixture was stirred for 35 hours under a nitrogen atmosphere. Then it was distributed between water and diethyl ether, the ethereal phase was dried over sodium sulphate, evaporated down to a volume of approx. 200 ml and combined with ethereal hydrogen chloride solution until the reaction of precipitation had ended. The colourless crystals obtained were suction filtered, washed with diethyl ether and dried in the air.

Yield: 22.0 g (87% of theory). mp. 215-225° C.

### EXAMPLE A27

Methyl 2-(1-piperazinyl)-thiazole-5-carboxylate

10.0 g (116.09 mmol) of anhydrous piperazine were added to a solution of 4.2 g (23.647 mmol) of methyl 2-chlorothiazole-5-carboxylate in 5 ml of ethanol and refluxed for 3 hours. The reaction mixture was combined with saturated aqueous sodium hydrogen carbonate solution and exhaustively extracted with ethyl acetate. The combined organic extracts were washed thoroughly with water, dried over sodium sulphate and concentrated by evaporation in vacuo. 1.8 g (34% of theory) of colourless crystals were obtained, R_{f} 0.44 (EI D).

### EXAMPLE A28

Methyl 2-chlorothiazole-5-carboxylate

20 g of crushed ice were added to a suspension of 14.0 g (71.927 mmol) of methyl 2-aminothiazol-5-carboxylate hydrochloride in 8 ml of conc. hydrochloric acid and while cooling externally a solution of 5.0 g (72.464 mmol) of sodium nitrite in 30 ml of water was added dropwise, while the reaction temperature was kept below 0° C. at all times. After 30 minutes 7.2 g (72.735 mmol) of copper (I) chloride were added, the mixture was stirred for another hour while being cooled and in the following 1½ hours allowed to come slowly up to room temperature. The mixture was exhaustively extracted with diethyl ether, the combined extracts were washed with saturated saline solution, dried over sodium sulphate and evaporated down. 4.3 g (34% of theory) of a colourless oil were obtained, R_{f}=0.94 (EI D), which was used in the next steps without any further purification.

MS: M⁺=177/179 (Cl)

### EXAMPLE A29

Methyl 2-(1-piperazinyl)-thiazole-4-carboxylate hydrochloride

4.0 ml (35.973 mmol) of 1-chloroethyl chloroformate were added to an ice-cooled solution of 8.0 g (15.752 mmol) of methyl 2-[4-(phenylmethyl)-1-piperazinyl]-thiazole-4-carboxylate in 60 ml of 1,2-dichloroethane, the mixture was stirred for another 20 minutes at 0° C. and refluxed overnight, before distilling off the solvent. The residue was combined with 60 ml of methanol and refluxed for another 4 hours. The solvent was eliminated in vacuo, the residue was triturated with 3 ml of methanol, then suction filtered. After drying in the vacuum drying cupboard 2.5 g (60% of theory) of colourless crystals were obtained, R_{f}=0.49 (EI D).

ESI-MS: (M+H)⁺=228; (M+Na)⁺=250

### EXAMPLE A30

2-[4-(phenylmethyl)-1-piperazinyl]-thiazole-4-carboxylic acid-hydrobromide

12.7 g (76.066 mmol) of bromopyruvic acid were added to the solution of 18.0 g (76.482 mmol) of 1-(aminothiocarbonyl)-4-(phenylmethyl)-piperazine in 300 ml of ethanol and refluxed for 3 hours. The mixture was left to stand overnight, the precipitated solid product was separated off by suction filtering and washed with ethanol. After drying 23.0 g (79% of theory) of colourless crystals were obtained, R_{f} 0.10 (EI D).

ESI-MS: (M−H)⁻=302; (M+Na)⁺=326

### EXAMPLE A31

1-(aminothiocarbonyl)-4-(phenylmethyl)-piperazine

12.596 g (108.247 mmol) of tert.-butyl isothiocyanate were added dropwise to an ice-cooled solution of 19.08 g (108.25 mmol) of 1-(phenylmethyl)-piperazine in 150 ml dichloromethane, while keeping the reaction temperature below +5° C. The mixture was stirred overnight at room temperature, freed from solvent and the residue remaining was boiled for 1½ hours with 100 ml of conc. hydrochloric acid. After cooling, it was neutralised while cooling externally with 12M sodium hydroxide solution and extracted exhaustively with dichloromethane. The combined dichloromethane extracts were dried over sodium sulphate and concentrated by evaporation in vacuo. 25.2 g (99% of theory) of bright yellow crystals were obtained, R_{f}=0.45 (EI D).

ESI-MS: (M+H)⁺=236; (M−H)⁻=234; (M+Na)⁺=258

### EXAMPLE A32

Ethyl 4-methyl-1-(phenylmethyl)-2-piperazinecarboxylate

A solution of 2.2 ml (35.029 mmol) of iodomethane in 50 ml THF was added dropwise at room temperature to a mixture of 15.12 g (31.739 mmol) of ethyl 1-(phenylmethyl)-2-piperazinecarboxylate-bis-(trifluoroacetate), 20 ml DIEA and 250 ml THF and stirred for a further 4 hours at room temperature. The mixture was filtered, the residue was evaporated down in vacuo and chromatographed on a silica gel column using EI II as eluant. After the appropriate fractions had been worked up in the usual way, 2.43 g (29% of theory) of a colourless oil were obtained, which was used in the next steps without further purification.

The following compounds of general formula N-B-C were prepared analogously:

| | | | | | | | | |
|---|---|---|---|---|---|---|---|---|
| | | | | % | | | | |
| N | B | C | Remarks | yield | EI | R_{f} | MS | mp. [° C.] |

| | | | | | | |
|---|---|---|---|---|---|---|
| ethyl 4-(1,1- | from ethyl 4-(1,1- | 79 | AcOEt | 0.58 | ESI: | colourless |
| dimethylethoxycarbonyl)- | dimethylethoxy- | | | | (M + H)⁺ = 273 | oil |
| 1-methyl-2- | carbonyl)-2- | | | | | |
| piperazinecarboxylate | piperazinecarboxylate, | | | | | |
| | CH₃l and DIEA in | | | | | |
| | THF | | | | | |
| ethyl 4-(1,1- | from ethyl 4-(1,1- | 90 | NN | 0.51 | ESI: | |
| dimethylethoxycarbonyl)- | dimethylethoxy- | | | | (M + H)⁺ = 349 | |
| 1-(phenylmethyl)-2- | carbonyl)-2- | | | | | |
| piperazinecarboxylate | piperazinecarboxylate, | | | | | |
| | PhCH₂Br and DIEA in | | | | | |
| | THF | | | | | |

### EXAMPLE A33

Ethyl 4-(1,1-dimethylethoxycarbonyl)-2-piperazinecarboxylate

22.0 g (0.101 mol) di-tert.-butyl pyrocarbonate were added dropwise to a solution of 17.07 g (0.108 mol) ethyl 2-piperazinecarboxylate in 400 ml of ethanol while cooling with ice and the mixture was stirred for a further 3 hours while cooling externally with ice. The solvent was distilled off, lastly in vacuo, and the residue remaining was distributed between water and ethyl acetate. The organic phase was dried over sodium sulphate and evaporated down in vacuo, the residue was purified by column chromatography on silica gel using ethyl acetate/ethanol 95/5 v/v as eluant.

Yield: 11.798 g (42% of theory) of a colourless solid.

### EXAMPLE A34

Ethyl 1,4-bis-(phenylmethyl)-2-piperazinecarboxylate

A solution of 56.441 g (217.141 mmol) of ethyl 2,3-dibromopropanoate in 55 ml of toluene was added dropwise to a solution, heated to 40° C., of 52.190 g (217.141 mmol) of N,N′-dibenzylethylenediamine and 60 ml triethylamine in 165 ml of toluene, with vigorous stirring, and stirred for a further 3 hours at a bath temperature of 80° C. The mixture was left to cool, filtered, the filtrates were washed twice with 50 ml of water, then once with 100 ml of saturated saline solution, dried over sodium sulphate and evaporated down in vacuo. 73.4 g (100% of theory) of a colourless viscous oil were obtained, R_{f} 0.79 (EI MM), which was used without further purification in the following step.

ESI-MS: (M+H)⁺=339

B. Preparation of the Final Compounds

### EXAMPLE 1

Ethyl 4-{1-[3,5-dibromo-N-[[4-(3,4-dihydro-2(1H)-oxoquinazolin-3-yl)-1-piperidinyl]carbonyl]-D-tyrosyl]-4-piperidinyl}-1-piperazineacetate (Ser. no.1)

The mixture of 954.048 mg (1.6 mmol) 3,5-dibromo-N-[[4-(3,4-dihydro-2(1H)-oxoquinazolin-3-yl)-1-piperidinyl]carbonyl]-D-tyrosine, 955.898 mg (1.6 mmol) ethyl 4-(4-piperidinyl)-1-piperazin-acetate, 802.75 mg (2.5 mmol) TBTU, 216.208 mg (1.6 mmol) HOBt, 2.4 ml (14.02 mmol) DIEA and 8 ml THF-DMF-mixture (5/3 v/v) was stirred overnight at ambient temperature. The reaction mixture was stirred into 50 ml of saturated aqueous sodium hydrogen carbonate solution, the precipitated solid was purified by column chromatography on silica gel using EI G as eluant. After the eluates had been worked up in the usual way 283 mg (21% of theory) of a colourless amorphous product were obtained, R_{f} 0.39 (EI G).

IR (KBr): 3405(NH, OH); 1731 (C═O) cm⁻¹

ESI: (M−H)⁻=830/832/834(Br₂); (M+Na)⁺=854/8561858(Br₂)

The following compounds of general formula N-B-C were prepared analogously:

| Ser. | | | | | % | | | | IR | |
|---|---|---|---|---|---|---|---|---|---|---|
| no. | N | B | C | Remarks | yield | EI | R_{f} | MS | [cm⁻¹] | mp. [° C.] |
| 3 | N1 | B1 | C3 | from N1—CO—B1—OH, | 71 | G | 0.34 | ESI: (M − H)⁻ = 858/ | 1740 | colourless |
| | | | | H—C3, TBTU, HOBt | | | | 860/862 (Br₂); | (C═O) | amorphous |
| | | | | and DIEA in THF | | | | (M + Na)⁺ = 882/ | | substance |
| | | | | | | | | 884/886 (Br₂) | | |
| 5 | N1 | B1 | C5 | from N1—CO—B1—OH, | 56 | G | 0.36 | ESI: (M − H)⁻ = 815/ | | colourless |
| | | | | H—C5 * 2 CF₃CO₂H, | | | | 817/819 (Br₂); | | amorphous |
| | | | | TBTU, HOBt and | | | | (M + Na)⁺ = 839/ | | substance |
| | | | | DIEA in THF | | | | 841/843 (Br₂) | | |
| 7 | N1 | B1 | C7 | from N1—CO—B1—OH, | 53 | G | 0.37 | ESI: (M − H)⁻ = 815/ | 3421 | colourless |
| | | | | H—C7, TBTU, HOBt | | | | 817/819 (Br₂); | broad | amorphous |
| | | | | and DIEA in THF | | | | (M + H)⁺ = 817/ | (NH, | substance |
| | | | | | | | | 819/821 | OH); | |
| | | | | | | | | (Br2);(M + Na)⁺ = 839/ | 1726 | |
| | | | | | | | | 841/843 (Br₂) | (C═O) | |
| 9 | N1 | B1 | C9 | from N1—CO—B1—OH, | 46 | G | 0.40 | ESI: (M − H)⁻ = 815/ | | colourless |
| | | | | H—C9, TBTU, HOBt | | | | 817/819 (Br₂); | | amorphous |
| | | | | and DIEA in THF | | | | (M + H)⁺ = 817/ | | substance |
| | | | | | | | | 819/821 (Br₂) | | |
| 11 | N1 | B1 | C11 | from N1—CO—B1—OH, | 51 | G | 0.32 | ESI: (M − H)⁻ = 830/ | 3317 | colourless |
| | | | | H—C11, TBTU, HOBt | | | | 832/834 (Br₂) | broad | amorphous |
| | | | | and DIEA in THF | | | | | (NH, | substance |
| | | | | | | | | | OH); | |
| | | | | | | | | | 1738 | |
| | | | | | | | | | (C═O) | |
| 12 | N2 | B2 | C5 | from N2—CO—B2—OH, | 96 | G | 0.61 | ESI: (M + H)+ = 830/ | 3377 | colourless |
| | | | | H—C5, TBTU, HOBt | | | | 832/834; | broad | amorphous |
| | | | | and DIEA in THF | | | | (M + HCO₂)⁻ = 874/ | (NH, | substance |
| | | | | | | | | 876/878 (Br₂) | NH₂); | |
| | | | | | | | | | 1734 | |
| | | | | | | | | | (C═O) | |
| 14 | N2 | B2 | C11 | from N2—CO—B2—OH, | 82 | G | 0.57 | ESI: (M + HCO₂)⁻ = 889/ | 3446 | colourless |
| | | | | H—C11, TBTU, HOBt | | | | 891/893 (Br₂) | broad | amorphous |
| | | | | and DIEA in THF | | | | | (NH, | substance |
| | | | | | | | | | NH₂); | |
| | | | | | | | | | 1734 | |
| | | | | | | | | | (C═O) | |
| 15 | N1 | B3 | C1 | from N1—CO—B3—OH, | 26 | | | ESI: (M + H)⁺ = 766/ | 1669 | |
| | | | | H—C1 * 3 CF₃CO₂H, | | | | 768 (Br) | (C═O) | |
| | | | | TBTU, HOBt and | | | | | | |
| | | | | DIEA in DMF | | | | | | |
| | | | | (Chemspeed) | | | | | | |
| 16 | N1 | B4 | C1 | from N1—CO—B4—OH, | 24 | | | ESI: (M + H)⁺ = 742/ | | |
| | | | | H—C1 * 3 CF₃CO₂H, | | | | 744/746 (Cl₂) | | |
| | | | | TBTU, HOBt and | | | | | | |
| | | | | DIEA in DMF | | | | | | |
| | | | | (Chemspeed) | | | | | | |
| 17 | N1 | B5 | C1 | from N1—CO—B5—OH, | 37 | | | ESI: (M + H)⁺ = 816/ | | |
| | | | | H—C1 * 3 CF₃CO₂H, | | | | 818/820 (Br₂) | | |
| | | | | TBTU, HOBt and | | | | | | |
| | | | | DIEA in DMF | | | | | | |
| | | | | (Chemspeed) | | | | | | |
| 18 | N1 | B6 | C1 | from N1—CO—B6—OH, | 26 | | | ESI: (M + Na)⁺ = 788/ | | |
| | | | | H—C1 * 3 CF₃CO₂H, | | | | 790 (Br) | | |
| | | | | TBTU, HOBt and | | | | | | |
| | | | | DIEA in DMF | | | | | | |
| | | | | (Chemspeed) | | | | | | |
| 19 | N1 | B7 | C1 | from N1—CO—B7—OH, | 18 | | | ESI: (M + Na)⁺ = 852/ | | |
| | | | | H—C1 * 3 CF₃CO₂H, | | | | 854/856 (Br₂) | | |
| | | | | TBTU, HOBt and | | | | | | |
| | | | | DIEA in DMF | | | | | | |
| | | | | (Chemspeed) | | | | | | |
| 20 | N1 | B8 | C1 | from N1—CO—B8—OH, | 13 | | | ESI: (M + H)⁺ = 708/ | | |
| | | | | H—C1 * 3 CF₃CO₂H, | | | | 710 (Cl) | | |
| | | | | TBTU, HOBt and | | | | | | |
| | | | | DIEA in DMF | | | | | | |
| | | | | (Chemspeed) | | | | | | |
| 21 | N1 | B3 | C11 | from N1—CO—B3—OH, | 26 | | | ESI: (M + Na)⁺ = 788/ | | |
| | | | | H—C11, TBTU, HOBt | | | | 790 (Br) | | |
| | | | | and DIEA in DMF | | | | | | |
| | | | | (Chemspeed) | | | | | | |
| 29 | N1 | B9 | C12 | from N1—CO—B9—OH, | 40 | | | ESI: (M + H)⁺ = 724 | | |
| | | | | H—C12, TBTU, HOBt | | | | | | |
| | | | | and DIEA in DMF | | | | | | |
| | | | | (Chemspeed) | | | | | | |
| 30 | N1 | B10 | C5 | from N1—CO—B10— | 66 | G | 0.35 | ESI: (M + H)⁺ = 661 | 1662 | colourless |
| | | | | OH, H—C5, TBTU, | | | | | (C═O) | amorphous |
| | | | | HOBt and DIEA in | | | | | | substance |
| | | | | DMF (Chemspeed) | | | | | | |
| 31 | N1 | B10 | C1 | from N1—CO—B10— | 22 | | | ESI: (M + H)⁺ = 676 | 1734, | colourless |
| | | | | OH, H—C1, TBTU, | | | | | 1660 | amorphous |
| | | | | HOBt and DIEA in | | | | | (C═O) | substance |
| | | | | DMF (Chemspeed) | | | | | | |
| 32 | N1 | B21 | C1 | from N1—CO—B21— | 13 | | | ESI: (M − H)⁻ = 827/ | 1670 | colourless |
| | | | | OH, H—C1, TBTU | | | | 829/831 (Br₂); | (C═O) | amorphous |
| | | | | and NEt₃ in | | | | (M + H)⁺ = 829/ | | substance |
| | | | | THF/DMF (10/1 v/v) | | | | 831/833 (Br₂) | | |
| 33 | N1 | B2 | C14 | from N1—CO—B2—OH, | 33 | S | 0.67 | ESI: (M + H)⁺ = 788/ | 3435, | 184.6 |
| | | | | H—C14, TBTU and | | | | 790/792 (Br₂); | 3373 | |
| | | | | NEt₃ in THF/DMF | | | | (M + Na)⁺ = 810/ | (NH, | |
| | | | | (1/1 v/v) | | | | 812/814 (Br₂) | NH₂); | |
| | | | | | | | | | 1734, | |
| | | | | | | | | | 1668 | |
| | | | | | | | | | (C═O) | |
| 34 | N1 | B1 | C14 | from N1—CO—B1—OH, | 6 | S | 0.67 | ESI: (M − H)⁻ = 787/ | 1653 | 141.9 |
| | | | | H—C14, TBTU and | | | | 789/791 (Br₂); | (C═O) | |
| | | | | NEt₃ in THF/DMF | | | | (M + H)⁺ = 789/ | | |
| | | | | (1/1 v/v) | | | | 791/793 (Br₂) | | |
| 37 | N1 | B2 | C16 | from N1—CO—B2—OH, | 53 | S | 0.67 | ESI: (M + H)⁺ = 788/ | 3437 | colourless |
| | | | | H—C16, TBTU and | | | | 790/792 (Br₂) | (NH, | crystals |
| | | | | NEt₃ in THF/DMF | | | | | NH₂); | |
| | | | | (1/1 v/v) | | | | | 1653 | |
| | | | | | | | | | (C═O) | |
| 38 | N1 | B1 | C16 | from N1—CO—B1—OH, | 32 | S | 0.67 | ESI: (M + H)⁺ = 789/ | 3321 | colourless |
| | | | | H—C16, TBTU and | | | | 791/793 (Br₂) | (NH, | crystals |
| | | | | NEt₃ in THF/DMF | | | | | OH); | |
| | | | | (1/1 v/v) | | | | | 1662 | |
| | | | | | | | | | (C═O) | |
| 41 | N1 | B2 | C18 | from N1—CO—B2—OH, | 26 | G | 0.35 | ESI: (M + H)⁺ = 802/ | | colourless |
| | | | | H—C18 * AcOH, | | | | 804/806 (Br₂) | | crystals |
| | | | | TBTU and NEt₃ in | | | | | | |
| | | | | THF/DMF (1/1 v/v) | | | | | | |
| 42 | N1 | B1 | C18 | from N1—CO—B1—OH, | 35 | G | 0.47 | ESI: (M + H)⁺ = 803/ | | colourless |
| | | | | H—C18 * AcOH, | | | | 805/807 (Br₂) | | crystals |
| | | | | TBTU and NEt₃ in | | | | | | |
| | | | | THF/DMF (1/1 v/v) | | | | | | |
| 43 | N1 | B2 | C19 | from N1—CO—B2—OH, | 52 | Q | 0.73 | ESI: (M + H)⁺ = 802/ | | colourless |
| | | | | H—C19, TBTU and | | | | 804/806 (Br₂); | | crystals |
| | | | | NEt₃ in THF/DMF | | | | (M + Na)⁺ = 824/ | | |
| | | | | (1/1 v/v) | | | | 826/828 (Br₂) | | |
| 44 | N1 | B1 | C19 | from N1—CO—B1—OH, | 63 | Q | 0.72 | ESI: (M + H)⁺ = 803/ | | colourless |
| | | | | H—C19, TBTU and | | | | 805/807 (Br₂) | | crystals |
| | | | | NEt₃ in THF/DMF | | | | | | |
| | | | | (1/1 v/v) | | | | | | |
| 49 | N1 | B1 | C22 | from N1—CO—B1—OH, | 49 | G | 0.44 | ESI: (M − H)⁻ = 801/ | | colourless |
| | | | | H—C22, TBTU and | | | | 803/805 (Br₂) | | crystals |
| | | | | NEt₃ in THF/DMF | | | | | | |
| | | | | (1/1 v/v) | | | | | | |
| 50 | N1 | B2 | C22 | from N1—CO—B2—OH, | 70 | G | 0.65 | ESI: (M + H)⁺ = 802/ | | colourless |
| | | | | H—C22, TBTU and | | | | 804/806 (Br₂) | | crystals |
| | | | | NEt₃ in THF/DMF | | | | | | |
| | | | | (1/1 v/v) | | | | | | |
| 55 | N1 | B1 | C26 | from N1—CO—B1—OH, | 52 | D | 0.55 | ESI: (M − H)⁻ = 809/ | | colourless |
| | | | | H—C26, TBTU, HOBt | | | | 811/813 (Br₂) | | crystals |
| | | | | and DIEA in THF | | | | | | |
| 56 | N1 | B1 | C27 | from N1—CO—B1—OH, | 54 | D | 0.56 | ESI: (M − H)⁻ = 809/ | | colourless |
| | | | | H—C27 * 2 HBr, | | | | 811/813 (Br₂) | | crystals |
| | | | | TBTU, HOBt and | | | | | | |
| | | | | DIEA in THF | | | | | | |
| 57 | N1 | B1 | C28 | from N1—CO—B1—OH, | 33 | D | 0.56 | ESI: (M − H)⁻ = 794/ | | colourless |
| | | | | H—C28, TBTU, HOBt | | | | 796/798 (Br₂) | | crystals |
| | | | | and DIEA in THF | | | | | | |
| 58 | N1 | B1 | C29 | from N1—CO—B1—OH, | 32 | D | 0.57 | ESI: (M − H)⁻ = 822/ | | colourless |
| | | | | H—C29, TBTU, HOBt | | | | 824/826 (Br₂) | | crystals |
| | | | | and DIEA in THF | | | | | | |
| 59 | N1 | B1 | C30 | from N1—CO—B1—OH, | 25 | D | 0.68 | ESI: (M − H)⁻ = 836/ | 1716, | colourless |
| | | | | H—C30, TBTU, HOBt | | | | 838/840 (Br₂); | 1662 | crystals |
| | | | | and DIEA in THF | | | | (M + Na)⁺ = 860/ | (C═O) | |
| | | | | | | | | 862/864 (Br₂) | | |
| 60 | N1 | B1 | C31 | from N1—CO—B1—OH, | 55 | D | 0.59 | ESI: (M − H)⁻ = 863/ | | colourless |
| | | | | H—C31 * 2 HCl, | | | | 865/867 (Br₂) | | crystals |
| | | | | TBTU, HOBt and | | | | | | |
| | | | | DIEA in THF | | | | | | |
| 61 | N1 | B1 | C32 | from N1—CO—B1—OH, | 45 | D | 0.59 | ESI: (M − H)⁻ = 794/ | | colourless |
| | | | | H—C32 * HCl, TBTU, | | | | 796/798 (Br₂); | | crystals |
| | | | | HOBt and DIEA in | | | | (M + Na)⁺ = 818/ | | |
| | | | | THF | | | | 820/822 (Br₂) | | |
| 62 | N2 | B2 | C26 | from N2—CO—B2—OH, | 62 | D | 0.81 | ESI: (M − H)⁻ = 822/ | | colourless |
| | | | | H—C26, TBTU, HOBt | | | | 824/826 (Br₂); | | crystals |
| | | | | and DIEA in THF | | | | (M + Na)⁺ = 846/ | | |
| | | | | | | | | 848/850 (Br₂) | | |
| 63 | N2 | B2 | C27 | from N2—CO—B2—OH, | 65 | D | 0.79 | ESI: (M + Na)⁺ = 846/ | | colourless |
| | | | | H—C27 * 2 HBr, | | | | 848/850 (Br₂) | | crystals |
| | | | | TBTU, HOBt and | | | | | | |
| | | | | DIEA in THF | | | | | | |
| 64 | N2 | B2 | C28 | from N2—CO—B2—OH, | 38 | D | 0.81 | ESI: (M − H)⁻ = 807/ | | colourless |
| | | | | H—C28, TBTU, HOBt | | | | 809/811 (Br₂) | | crystals |
| | | | | and DIEA in THF | | | | | | |
| 65 | N2 | B2 | C30 | from N2—CO—B2—OH, | 54 | D | 0.87 | ESI: (M + Na)⁺ = 873/ | | colourless |
| | | | | H—C30, TBTU, HOBt | | | | 875/877 (Br₂) | | crystals |
| | | | | and DIEA in THF | | | | | | |
| 66 | N2 | B2 | C31 | from N2—CO—B2—OH, | 50 | D | 0.85 | ESI: (M + Na)⁺ = 900/ | | colourless |
| | | | | H—C31 * 2 HCl, | | | | 902/904 (Br₂) | | crystals |
| | | | | TBTU, HOBt and | | | | | | |
| | | | | DIEA in THF | | | | | | |
| 67 | N2 | B2 | C32 | from N2—CO—B2—OH, | 52 | D | 0.88 | ESI: (M − H)⁻ = 807/ | 1723 | colourless |
| | | | | H—C32 * HCl, TBTU, | | | | 809/811 (Br₂); | (C═O) | crystals |
| | | | | HOBt and DIEA in | | | | (M + Na)⁺ = 831/ | | |
| | | | | THF | | | | 833/835 (Br₂) | | |
| 83 | N1 | B1 | C40 | from N1—CO—B1—OH, | 17 | D | 0.50 | ESI: (M − H)⁻ = 802/ | | colourless |
| | | | | H—C40, TBTU, HOBt | | | | 804/806 (Br₂); | | crystals |
| | | | | and DIEA in THF | | | | (M + Na)⁺ = 826/ | | |
| | | | | | | | | 828/830 (Br₂) | | |
| 84 | N1 | B1 | C41 | from N1—CO—B1—OH, | 82 | D | 0.41 | ESI: (M − H)⁻ = 802/ | | |
| | | | | H—C41 * HCl, | | | | 804/806 (Br₂) | | |
| | | | | TBTU, HOBt and | | | | | | |
| | | | | DIEA in THF | | | | | | |
| 87 | N1 | B2 | C41 | from N1—CO—B2—OH, | 75 | D | 0.62 | ESI: (M − H)⁻ = 801/ | | |
| | | | | H—C41 * HCl, | | | | 803/805 (Br₂) | | |
| | | | | TBTU, HOBt and | | | | | | |
| | | | | DIEA in THF | | | | | | |
| 88 | N1 | B2 | C40 | from N1—CO—B2—OH, | 62 | D | 0.52 | ESI: (M + Na)⁺ = 825/ | | |
| | | | | H—C40, TBTU, HOBt | | | | 827/829 (Br₂) | | |
| | | | | and DIEA in THF | | | | | | |
| 93 | N1 | B2 | C12 | from N1—CO—B2—OH, | 55 | D | 0.47 | ESI: (M − H)⁻ = 857/ | 1665 | colourless |
| | | | | H—C12, TBTU, HOBt | | | | 859/861 (Br₂); | (C═O) | crystals |
| | | | | and DIEA in THF | | | | (M + H)⁺ = 859/ | | |
| | | | | | | | | 861/863 (Br₂); | | |
| | | | | | | | | (M + Na)⁺ = 881/ | | |
| | | | | | | | | 883/885 (Br₂) | | |
| 94 | N2 | B2 | C12 | from N2—CO—B2—OH, | 65 | D | 0.49 | ESI: (M − H)⁻ = 871/ | | colourless |
| | | | | H—C12, TBTU, HOBt | | | | 873/875 (Br₂); | | crystals |
| | | | | and DIEA in THF | | | | (M + Na)⁺ = 895/ | | |
| | | | | | | | | 897/899 (Br₂) | | |
| 95 | N1 | B2 | C1 | from N1—CO—B2—OH, | 57 | D | 0.68 | ESI: (M + H)⁺ = 831/ | 1665 | colourless |
| | | | | H—C1, TBTU, HOBt | | | | 833/835 (Br₂) | (C═O) | crystals |
| | | | | and DIEA in THF | | | | | | |
| 96 | N2 | B2 | C1 | from N2—CO—B2—OH, | 58 | D | 0.72 | ESI: (M − H)⁻ = 843/ | 1658 | colourless |
| | | | | H—C1, TBTU, HOBt | | | | 845/847 (Br₂); | (C═O) | crystals |
| | | | | and DIEA in THF | | | | (M + H)⁺ = 845/ | | |
| | | | | | | | | 847/849 (Br₂) | | |
| 119 | N1 | B30 | C1 | from N1—CO—B30— | 50 | | | ESI: (M + H)⁺ = 815/ | | colourless |
| | | | | OH, H—C1, TBTU, | | | | 817/819 (Br₂) | | crystals |
| | | | | HOBt and DIEA in | | | | | | |
| | | | | THF | | | | | | |
| 122 | N1 | B7 | C14 | from N1—CO—B7—OH, | 26 | II | 0.44 | ESI: (M + H)⁺ = 787/ | | colourless |
| | | | | H—C14, TBTU and | | | | 789/791 (Br₂) | | amorphous |
| | | | | NEt₃ in DMF | | | | | | substance |
| 123 | N1 | B8 | C14 | from N1—CO—B8—OH, | 28 | C | 0.68 | ESI: (M + H)⁺ = 665/ | | highly |
| | | | | H—C14, TBTU and | | | | 667 (Cl) | | viscous oil |
| | | | | NEt₃ in DMF | | | | | | |
| 124 | N1 | B7 | C16 | from N1—CO—B7—OH, | 20 | C | 0.80 | ESI: (M + H)⁺ = 787/ | | highly |
| | | | | H—C16, TBTU and | | | | 789/791 (Br₂) | | viscous oil |
| | | | | NEt₃ in DMF | | | | | | |
| 125 | N1 | B8 | C16 | from N1—CO—B8—OH, | 11 | II | 0.58 | ESI: (M + H)⁺ = 665/ | | colourless |
| | | | | H—C16, TBTU and | | | | 667 (Cl) | | amorphous |
| | | | | NEt₃ in DMF | | | | | | substance |
| 128 | N1 | B32 | C45 | from N1—CO—B32— | 4 | C | 0.45 | ESI: (M + H)⁺ = 703 | | colourless |
| | | | | OH, H—C45, TBTU, | | | | | | solid |
| | | | | HOBt and NEt₃ in | | | | | | substance |
| | | | | DMF | | | | | | |
| 129 | N1 | B30 | C45 | from N1—CO—B30— | 19 | C | 0.72 | ESI: (M + H)⁺ = 815/ | | colourless |
| | | | | OH, H—C45, TBTU | | | | 817/819 (Br₂) | | solid |
| | | | | and DIEA in THF | | | | | | substance |
| 130 | N1 | B30 | C44 | from N1—CO—B30— | 18 | C | 0.81 | ESI: (M + H)⁺ = 815/ | | colourless |
| | | | | OH, H—C44, TBTU | | | | 817/819 (Br₂) | | solid |
| | | | | and DIEA in THF | | | | | | substance |
| 131 | N1 | B21 | C45 | from N1—CO—B21— | 14 | C | 0.67 | ESI: (M + H)⁺ = 829/ | | colourless |
| | | | | OH, H—C45, TBTU | | | | 831/833 (Br₂) | | solid |
| | | | | and DIEA in THF | | | | | | substance |
| 132 | N1 | B21 | C44 | from N1—CO—B21— | 24 | C | 0.48 | ESI: (M + H)⁺ = 829/ | | colourless |
| | | | | OH, H—C44, TBTU | | | | 831/833 (Br₂) | | solid |
| | | | | and DIEA in THF | | | | | | substance |
| 133 | N1 | B30 | C46 | from N1—CO—B30— | 16 | C | 0.55 | ESI: (M + H)⁺ = 815/ | | colourless |
| | | | | OH, H—C46, TBTU | | | | 817/819 (Br₂) | | solid |
| | | | | and DIEA in THF | | | | | | substance |
| 138 | N1 | B21 | C46 | from N1—CO—B21— | 26 | Q | 0.65 | ESI: (M + H)⁺ = 829/ | | colourless |
| | | | | OH, H—C46, PyBroP | | | | 831/833 (Br₂) | | solid |
| | | | | and DIEA in THF | | | | | | substance |
| 140 | N1 | B31 | C44 | from N1—CO—B31— | 22 | Q | 0.57 | ESI: (M + H)⁺ = 830/ | | colourless |
| | | | | OH, H—C44, PyBroP | | | | 832/834 (Br₂) | | solid |
| | | | | and DIEA in THF | | | | | | substance |
| 141 | N1 | B31 | C46 | from N1—CO—B31— | 15 | Q | 0.47 | ESI: (M + H)⁺ = 830/ | | colourless |
| | | | | OH, H—C46, PyBroP | | | | 832/834 (Br₂) | | solid |
| | | | | and DIEA in THF | | | | | | substance |
| 142 | N1 | B31 | C45 | from N1—CO—B31— | 11 | Q | 0.59 | ESI: (M + H)⁺ = 830/ | | colourless |
| | | | | OH, H—C45, PyBroP | | | | 832/834 (Br₂) | | solid |
| | | | | and DIEA in THF | | | | | | substance |
| 148 | N1 | B32 | C44 | from N1—CO—B32— | 24 | Q | 0.50 | ESI: (M + H)⁺ = 703 | 1736, | colourless |
| | | | | OH, H—C44, HATU | | | | | 1664, | solid |
| | | | | and DIEA in THF | | | | | 1637 | substance |
| | | | | | | | | | (C═O) | |
| 149 | N1 | B32 | C46 | from N1—CO—B32— | 3 | Q | 0.50 | M⁺ = 702 | | colourless |
| | | | | OH, H—C46, HATU | | | | | | solid |
| | | | | and DIEA in THF | | | | | | substance |
| 151 | N1 | B25 | C45 | from N1—CO—B25— | 10 | G | 0.38 | ESI: (M + H)⁺ = 805/ | | colourless |
| | | | | OH, H—C45, TBTU | | | | 807/809 (Cl₂) | | solid |
| | | | | and DIEA in THF | | | | | | substance |
| 152 | N1 | B30 | C50 | from N1—CO—B30— | 21 | G | 0.28 | ESI: (M + H)⁺ = 815/ | | colourless |
| | | | | OH, H—C50, TBTU | | | | 817/819 (Br₂) | | solid |
| | | | | and DIEA in THF | | | | | | substance |
| 153 | N1 | B21 | C50 | from N1—CO—B21— | 34 | G | 0.36 | ESI: (M + H)⁺ = 829/ | 3439 | colourless |
| | | | | OH, H—C50, TBTU | | | | 831/833 (Br₂) | (NH); | solid |
| | | | | and DIEA in THF | | | | | 1738, | substance |
| | | | | | | | | | 1666, | |
| | | | | | | | | | 1639 | |
| | | | | | | | | | (C═O) | |
| 154 | N1 | B32 | C50 | from N1—CO—B32— | 46 | G | 0.35 | ESI: (M + H)⁺ = 703 | 1736, | colourless |
| | | | | OH, H—C50, TBTU | | | | | 1660, | solid |
| | | | | and DIEA in THF | | | | | 1628 | substance |
| | | | | | | | | | (C═O) | |
| 155 | N1 | B31 | C50 | from N1—CO—B31— | 30 | Q | 0.66 | ESI: (M + H)⁺ = 830/ | 3458 | colourless |
| | | | | OH, H—C50, TBTU | | | | 832/834 (Br₂) | (NH, | solid |
| | | | | and DIEA in THF | | | | | NH₂); | substance |
| | | | | | | | | | 1734 | |
| | | | | | | | | | (C═O) | |
| 156 | N1 | B25 | C50 | from N1—CO—B25— | 29 | Q | 0.68 | ESI: (M + H)⁺ = 806/ | 3439 | colourless |
| | | | | OH, H—C50, TBTU | | | | 807/809/811 | (NH); | solid |
| | | | | and DIEA in THF | | | | (Br₂, Cl) | 1639 | substance |
| | | | | | | | | | (C═O) | |
| 162 | N1 | B5 | C45 | from N1—CO—B5—OH, | 22 | C | 0.69 | ESI: (M + H)⁺ = 816/ | | colourless |
| | | | | H—C45, TBTU and | | | | 818/820 (Br₂) | | solid |
| | | | | DIEA in THF/DMF | | | | | | substance |
| | | | | (3/1 v/v) | | | | | | |
| 164 | N1 | B33 | C5 | from N1—CO—B33— | 70 | C | 0.79 | ESI: (M + H)⁺ = 801/ | | colourless |
| | | | | OH, H—C5, TBTU | | | | 803/805 (Br₂) | | solid |
| | | | | and DIEA in THF | | | | | | substance |
| 166 | N1 | B7 | C45 | from N1—CO—B7—OH, | 25 | C | 0.69 | ESI: (M + H)⁺ = 830/ | 1738, | colourless |
| | | | | H—C45, TBTU and | | | | 832/834 (Br₂) | 1660 | solid |
| | | | | DIEA in THF/DMF | | | | | (C═O) | substance |
| 167 | N1 | B7 | C50 | from N1—CO—B7—OH, | 41 | C | 0.71 | ESI: (M + H)⁺ = 830/ | 1736, | colourless |
| | | | | H—C50, TBTU and | | | | 832/834 (Br₂) | 1662 | solid |
| | | | | DIEA in THF/DMF | | | | | (C═O) | substance |

### EXAMPLE 2

4-{1-[3,5-dibromo-N-[[4-(3,4-dihydro-2(1H)-oxoquinazolin-3-yl)-1-piperidinyl]carbonyl]-D-tyrosyl]-4-piperidinyl}-1-piperazine-acetic acid (Ser. no. 2)

0.5 ml of 1 M aqueous sodium hydroxide solution was added to a solution of 85.0 mg (0.102 mmol) of ethyl 4-{1-[3,5-dibromo-N-[[4-(3,4-dihydro-2(1H)-oxoquinazolin-3-yl)-1-piperidinyl]carbonyl]-D-tyrosyl]-4-piperidinyl}-1-piperazine-acetate in 3.5 ml of methanol at room temperature and the mixture was stirred for 1 hour at a reaction temperature of 40° C. The solvent was eliminated in vacuo and then neutralised while cooling externally with ice by the addition of 0.5 ml 1 M hydrochloric acid. The mixture was left to stand for 2 hours at room temperature before the precipitated crystals were collected. The mother liquor was evaporated down again, the residue was digested with a few drops of water to eliminate inorganic salts, left to stand for 2 hours and then filtered. The combined solids were dried in vacuo, triturated with diethyl ether and yielded 80.0 mg (97% of theory) of colourless crystals.

ESI-MS: (M+Na)⁺=826/828/830 (Br₂) (M−H)⁻=802/804/806 (Br₂)

The following compounds of general formula N-B-C were prepared analogously:

| Ser. | | | | | % | | | | | |
|---|---|---|---|---|---|---|---|---|---|---|
| no. | N | B | C | Remarks | yield | EI | R_{f} | MS | IR [cm⁻¹] | mp. [° C.] |
| 4 | N1 | B1 | C4 | from N1—CO—B1— | 88 | G | 0.02 | ESI: (M − H)⁻ = 802/ | | colourless |
| | | | | C3 with aq. 1M | | | | 804/806 (Br₂); | | crystals |
| | | | | NaOH, then aq. | | | | (M + Na)⁺ = 826/ | | |
| | | | | 1M HCl | | | | 828/830 (Br₂) | | |
| 6 | N1 | B1 | C6 | from N1—CO—B1— | 88 | G | 0.02 | ESI: (M − H)⁻ = 801/ | 3420 (NH, | colourless |
| | | | | C5 with aq. 1M | | | | 803/805 (Br₂); | OH), 1734, | crystals |
| | | | | NaOH, then aq. | | | | (M + H)⁺ = 803/ | 1653 (C═O) | |
| | | | | 1M HCl | | | | 805/807 (Br2); | | |
| | | | | | | | | (M + Na)⁺ = 825/ | | |
| | | | | | | | | 827/829 (Br₂) | | |
| 8 | N1 | B1 | C8 | from N1—CO—B1— | 96 | G | 0.02 | ESI: (M − H)⁻ = 787/ | 3420 (NH, | colourless |
| | | | | C7 with aq. 1M | | | | 789/791 (Br₂); | OH), 1709, | crystals |
| | | | | NaOH, then aq. | | | | (M + Na)⁺ = 811/ | 1653 (C═O) | |
| | | | | 1M HCl | | | | 813/815 (Br₂) | | |
| 10 | N1 | B1 | C10 | from N1—CO—B1— | 72 | G | 0.03 | ESI: (M − H)⁻ = 787/ | 3413 (NH, | colourless |
| | | | | C9 with aq. 1M | | | | 789/791 (Br₂); | OH), 1707, | crystals |
| | | | | NaOH, then aq. | | | | (M + Na)⁺ = 811/ | 1653 (C═O) | |
| | | | | 1M HCl | | | | 813/815 (Br₂) | | |
| 13 | N2 | B2 | C6 | from N2—CO—B2— | 78 | G | 0.04 | ESI: (M − H)⁻ = 814/ | 3431 (NH, | colourless |
| | | | | C5 with aq. 1M | | | | 816/818 (Br₂); | NH₂); 1653 | crystals |
| | | | | NaOH, then aq. | | | | (M + H)⁺ = 816/ | (C═O) | |
| | | | | 1M HCl | | | | 818/820 (Br2); | | |
| | | | | | | | | (M + HCO₂)⁻ = 859/ | | |
| | | | | | | | | 861/863 (Br₂) | | |
| 22 | N1 | B3 | C2 | from N1—CO—B3— | 97 | | | ESI: (M + H)⁺ = 738/ | 3425 (NH), | colourless |
| | | | | C1 with aq. 1M | | | | 740 (Br) | 1659, 1632 | crystals |
| | | | | NaOH, then aq. | | | | | (C═O) | |
| | | | | 1M HCl | | | | | | |
| 23 | N1 | B4 | C2 | from N1—CO—B4— | 99 | | | ESI: (M + Cl)⁻ = 748/ | 3419 (NH), | colourless |
| | | | | C1 with aq. 1M | | | | 750/752/754 | 1655, 1628 | crystals |
| | | | | NaOH, then aq. | | | | (Cl₂); (M + Na)⁺ = 736/ | (C═O) | |
| | | | | 1M HCl | | | | 738/740 (Cl₂) | | |
| 24 | N1 | B5 | C2 | from N1—CO—B5— | 98 | | | ESI: (M + Cl)⁻ = 822/ | 3419 (NH), | colourless |
| | | | | C1 with aq. 1M | | | | 824/826/828 | 1655, 1635 | crystals |
| | | | | NaOH, then aq. | | | | (Br₂); (M + Na)⁺ = 810/ | (C═O) | |
| | | | | 1M HCl | | | | 812/814 (Br₂) | | |
| 25 | N1 | B6 | C2 | from N1—CO—B6— | 98 | | | ESI: (M + Cl)⁻ = 772/ | 3427 (NH), | colourless |
| | | | | C1 with aq. 1M | | | | 774/776 (Br); | 1630 (C═O) | crystals |
| | | | | NaOH, then aq. | | | | (M + Na)⁺ = 760/ | | |
| | | | | 1M HCl | | | | 762 (Br) | | |
| 26 | N1 | B7 | C2 | from N1—CO—B7— | 99 | | | ESI: (M + Cl)⁻ = 836/ | 3419 (NH), | colourless |
| | | | | C1 with aq. 1M | | | | 838/840/842 | 1655, 1635 | crystals |
| | | | | NaOH, then aq. | | | | (Br₂); (M + Na)⁺ = 824/ | (C═O) | |
| | | | | 1M HCl | | | | 826/828 (Br₂) | | |
| 27 | N1 | B8 | C2 | from N1—CO—B8— | 89 | | | ESI: (M + Cl)⁻ = 714/ | 3419 (NH), | colourless |
| | | | | C1 with aq. 1M | | | | 716/718 (Cl); | 1655, 1635 | crystals |
| | | | | NaOH, then aq. | | | | (M + Na)⁺ = 702/ | (C═O) | |
| | | | | 1M HCl | | | | 704 (Cl) | | |
| 28 | N1 | B3 | C4 | from N1—CO—B3— | 97 | | | ESI: (M + Cl)⁻ = 772/ | 3416 (NH), | colourless |
| | | | | C11 with aq. 1M | | | | 774/776 (Br); | 1655, 1635 | crystals |
| | | | | NaOH, then aq. | | | | (M + Na)⁺ = 760/ | (C═O) | |
| | | | | 1M HCl | | | | 762 (Br) | | |
| 35 | N1 | B2 | C15 | from N1—CO—B2— | 78 | T | 0.46 | ESI: (M + Na)⁺ = 796/ | 3339 (NH, | colourless |
| | | | | C14 with aq. 1M | | | | 798/800 (Br₂) | NH₂); 1653 | crystals |
| | | | | LiOH, then aq. 1M | | | | | (C═O) | |
| | | | | HCl | | | | | | |
| 36 | N1 | B1 | C15 | from N1—CO—B1— | 78 | T | 0.42 | ESI: (M − H)⁻ = 773/ | | colourless |
| | | | | C14 with aq. 1M | | | | 775/779 (Br₂); | | crystals |
| | | | | LiOH, then aq. 1M | | | | (M + H)⁺ = 775/ | | |
| | | | | HCl | | | | 777/779 (Br₂); | | |
| | | | | | | | | (M + Na)⁺ = 797/ | | |
| | | | | | | | | 799/801 (Br₂) | | |
| 39 | N1 | B2 | C17 | from N1—CO—B2— | 76 | T | 0.46 | ESI: (M − H)⁻ = 772/ | 3429 (NH, | colourless |
| | | | | C16 with aq. 1M | | | | 774/776 (Br₂); | NH₂); 1653 | crystals |
| | | | | LiOH, then aq. 1M | | | | (M + Na)⁺ = 796/ | (C═O) | |
| | | | | HCl | | | | 798/800 (Br₂) | | |
| 40 | N1 | B1 | C17 | from N1—CO—B1— | 70 | T | 0.42 | ESI: (M − H)⁻ = 773/ | 3420 (NH, | colourless |
| | | | | C16 with aq. 1M | | | | 775/777 (Br₂); | OH); 1653 | crystals |
| | | | | LiOH, then aq. 1M | | | | (M + Na)⁺ = 797/ | (C═O) | |
| | | | | HCl | | | | 799/801 (Br₂) | | |
| 45 | N1 | B2 | C20 | from N1—CO—B2— | 96 | | | ESI: (M − H)⁻ = 786/ | | colourless |
| | | | | C18 with aq. 1M | | | | 788/790 (Br₂) | | crystals |
| | | | | LiOH, then aq. 1M | | | | | | |
| | | | | HCl | | | | | | |
| 46 | N1 | B1 | C20 | from N1—CO—B1— | 97 | | | ESI: (M − H)⁻ = 787/ | | colourless |
| | | | | C18 with aq. 1M | | | | 789/791 (Br₂) | | crystals |
| | | | | LiOH, then aq. 1M | | | | | | |
| | | | | HCl | | | | | | |
| 47 | N1 | B1 | C21 | from N1—CO—B1— | 86 | | | ESI: (M − H)⁻ = 787/ | | colourless |
| | | | | C19 with aq. 1M | | | | 789/791 (Br₂) | | crystals |
| | | | | LiOH, then aq. 1M | | | | | | |
| | | | | HCl | | | | | | |
| 48 | N1 | B2 | C21 | from N1—CO—B2— | 2 | | | ESI: (M − H)⁻ = 786/ | | colourless |
| | | | | C19 with aq. 1M | | | | 788/790 (Br₂); | | crystals |
| | | | | LiOH, then aq. 1M | | | | (M + Na)⁺ = 810/ | | |
| | | | | HCl | | | | 812/814 (Br₂) | | |
| 51 | N1 | B1 | C23 | from N1—CO—B1— | 12 | | | ESI: (M − H)⁻ = 787/ | | colourless |
| | | | | C22 with aq. 1M | | | | 789/791 (Br₂) | | amorphous |
| | | | | LiOH, then aq. 1M | | | | | | substance |
| | | | | HCl | | | | | | |
| 52 | N1 | B2 | C23 | from N1—CO—B2— | 14 | | | ESI: (M + H)⁺ = 788/ | | colourless |
| | | | | C22 with aq. 1M | | | | 790/792 (Br₂) | | amorphous |
| | | | | LiOH, then aq. 1M | | | | | | substance |
| | | | | HCl | | | | | | |
| 53 | N1 | B10 | C6 | from N1—CO—B10— | 36 | | | ESI: (M + H)⁺ = 647 | | colourless |
| | | | | C5 with aq. 1M | | | | | | amorphous |
| | | | | LiOH, then aq. | | | | | | substance |
| | | | | citric acid | | | | | | |
| 54 | N1 | B10 | C2 | from N1—CO—B10— | 21 | | | ESI: (M + H)⁺ = 648 | 1711, 1639 | colourless |
| | | | | C1 with aq. 1M | | | | | (C═O) | crystals |
| | | | | LiOH, then aq. | | | | | | |
| | | | | citric acid | | | | | | |
| 68 | N1 | B1 | C33 | from N1—CO—B1— | 77 | I | 0.51 | ESI: (M − H)⁻ = 781/ | 1655 (C═O) | colourless |
| | | | | C26 with aq. 1M | | | | 783/785 (Br₂) | | crystals |
| | | | | LiOH, then aq. 1M | | | | | | |
| | | | | HCl | | | | | | |
| 69 | N1 | B1 | C34 | from N1—CO—B1— | 75 | I | 0.50 | ESI: (M − H)⁻ = 781/ | 1637 (C═O) | colourless |
| | | | | C27 with aq. 1M | | | | 783/785 (Br₂); | | crystals |
| | | | | LiOH, then aq. 1M | | | | (M + Na)⁺ = 805/ | | |
| | | | | HCl | | | | 807/809 (Br₂) | | |
| 70 | N1 | B1 | C35 | from N1—CO—B1— | 82 | I | 0.52 | ESI: (M − H)⁻ = 780/ | | colourless |
| | | | | C28 with aq. 1M | | | | 782/784 (Br₂); | | crystals |
| | | | | LiOH, then aq. 1M | | | | (M + Na)⁺ = 804/ | | |
| | | | | HCl | | | | 806/808 (Br₂) | | |
| 71 | N1 | B1 | C36 | from N1—CO—B1— | 76 | I | 0.54 | ESI: (M − H)⁻ = 794/ | 1658 (C═O) | colourless |
| | | | | C29 with aq. 1M | | | | 796/798 (Br₂); | | crystals |
| | | | | LiOH, then aq. 1M | | | | (M + Na)⁺ = 818/ | | |
| | | | | HCl | | | | 820/822 (Br₂) | | |
| 72 | N1 | B1 | C37 | from N1—CO—B1— | 75 | I | 0.53 | ESI: (M − H)⁻ = 808/ | 1707, 1659 | colourless |
| | | | | C30 with aq. 1M | | | | 810/812 (Br₂); | (C═O) | crystals |
| | | | | LiOH, then aq. 1M | | | | (M + Na)⁺ = 832/ | | |
| | | | | HCl | | | | 834/836 (Br₂) | | |
| 73 | N1 | B1 | C38 | from N1—CO—B1— | 73 | I | 0.47 | ESI: (M − H)⁻ = 849/ | | colourless |
| | | | | C31 with aq. 1M | | | | 851/853 (Br₂); | | crystals |
| | | | | LiOH, then aq. 1M | | | | (M + Na)⁺ = 873/ | | |
| | | | | HCl | | | | 875/877 (Br₂) | | |
| 74 | N1 | B1 | C39 | from N1—CO—B1— | 68 | I | 0.49 | ESI: (M − H)⁻ = 780/ | 1711, 1657 | colourless |
| | | | | C32 with aq. 1M | | | | 782/784 (Br₂) | (C═O) | crystals |
| | | | | LiOH, then aq. 1M | | | | | | |
| | | | | HCl | | | | | | |
| 75 | N2 | B2 | C33 | from N2—CO—B2— | 82 | I | 0.55 | ESI: (M − H)⁻ = 794/ | | colourless |
| | | | | C26 with aq. 1M | | | | 796/798 (Br₂); | | crystals |
| | | | | LiOH, then aq. 1M | | | | (M + Na)⁺ = 818/ | | |
| | | | | HCl | | | | 820/822 (Br₂) | | |
| 76 | N2 | B2 | C34 | from N2—CO—B2— | 76 | I | 0.54 | ESI: (M − H)⁻ = 794/ | 1709, 1637 | colourless |
| | | | | C27 with aq. 1M | | | | 796/798 (Br₂); | (C═O) | crystals |
| | | | | LiOH, then aq. 1M | | | | (M + Na)⁺ = 818/ | | |
| | | | | HCl | | | | 820/822 (Br₂) | | |
| 77 | N2 | B2 | C35 | from N2—CO—B2— | 76 | I | 0.54 | ESI: (M − H)⁻ = 793/ | 1657 (C═O) | colourless |
| | | | | C28 with aq. 1M | | | | 795/797 (Br₂); | | crystals |
| | | | | LiOH, then aq. 1M | | | | (M + Na)⁺ = 817/ | | |
| | | | | HCl | | | | 819/821 (Br₂) | | |
| 78 | N2 | B2 | C37 | from N2—CO—B2— | 86 | I | 0.56 | ESI: (M − H)⁻ = 821/ | | colourless |
| | | | | C30 with aq. 1M | | | | 823/825 (Br₂); | | crystals |
| | | | | LiOH, then aq. 1M | | | | (M + Na)⁺ = 845/ | | |
| | | | | HCl | | | | 847/849 (Br₂) | | |
| 79 | N2 | B2 | C38 | from N2—CO—B2— | 77 | I | 0.56 | ESI: (M − H)⁻ = 862/ | | colourless |
| | | | | C31 with aq. 1M | | | | 864/866 (Br₂); | | crystals |
| | | | | LiOH, then aq. 1M | | | | (M + Na)⁺ = 886/ | | |
| | | | | HCl | | | | 888/890 (Br₂) | | |
| 80 | N2 | B2 | C39 | from N2—CO—B2— | 71 | I | 0.57 | ESI: (M − H)⁻ = 793/ | 1711 (C═O) | colourless |
| | | | | C32 with aq. 1M | | | | 795/797 (Br₂) | | crystals |
| | | | | LiOH, then aq. 1M | | | | | | |
| | | | | HCl | | | | | | |
| 82 | N2 | B11 | C2 | from N2—CO—B11— | 83 | | | ESI: (M + H)⁺ = 696 | | colourless |
| | | | | C1 with aq. 0.1M | | | | | | amorphous |
| | | | | LiOH, then aq. | | | | | | substance |
| | | | | 0.1M HCl | | | | | | |
| 85 | N1 | B1 | C42 | from N1—CO—B1— | 97 | O | 0.12 | ESI: (M − H)⁻ = 788/ | | colourless |
| | | | | C40 with aq. 0.1M | | | | 790/792 (Br₂) | | crystals |
| | | | | LiOH, then aq. | | | | | | |
| | | | | 0.1M HCl | | | | | | |
| 86 | N1 | B1 | C43 | from N1—CO—B1— | 82 | O | 0.16 | ESI: (M − H)⁻ = 788/ | | colourless |
| | | | | C41 with aq. 0.1M | | | | 790/792 (Br₂) | | crystals |
| | | | | LiOH, then aq. | | | | | | |
| | | | | 0.1M HCl | | | | | | |
| 89 | N1 | B2 | C43 | from N1—CO—B2— | 76 | D | 0.15 | ESI: (M − H)⁻ = 787/ | | colourless |
| | | | | C41 with aq. 0.1M | | | | 789/791 (Br₂) | | crystals |
| | | | | LiOH, then aq. | | | | | | |
| | | | | 0.1M HCl | | | | | | |
| 90 | N1 | B2 | C42 | from N1—CO—B2— | 86 | D | 0.16 | ESI: (M − H)⁻ = 787/ | | colourless |
| | | | | C40 with aq. 0.1M | | | | 789/791 (Br₂) | | crystals |
| | | | | LiOH, then aq. | | | | | | |
| | | | | 0.1M HCl | | | | | | |
| 91 | N1 | B2 | C4 | from N1—CO—B2— | 86 | M | 0.24 | ESI: (M − H)⁻ = 801/ | 1653 (C═O) | colourless |
| | | | | C11 with aq. 0.1M | | | | 803/805 (Br₂); | | crystals |
| | | | | LiOH, then aq. | | | | (M + H)⁺ = 803/ | | |
| | | | | 0.1M HCl | | | | 805/807 (Br₂) | | |
| 92 | N2 | B2 | C4 | from N2—CO—B2— | 69 | M | 0.31 | ESI: (M − H)⁻ = 815/ | | colourless |
| | | | | C11 with aq. 0.1M | | | | 817/819 (Br₂); | | crystals |
| | | | | LiOH, then aq. | | | | (M + Na)⁺ = 839/ | | |
| | | | | 0.1M HCl | | | | 841/843 (Br₂) | | |
| 97 | N1 | B2 | C2 | from N1—CO—B2— | 61 | D | 0.06 | ESI: (M − H)⁻ = 801803/ | 1653 (C═O) | colourless |
| | | | | C1 with aq. 1M | | | | 805 (Br₂); | | crystals |
| | | | | LiOH, then aq. 1M | | | | (M + Na)⁺ = 825/ | | |
| | | | | HCl | | | | 827/829 (Br₂) | | |
| 98 | N2 | B2 | C2 | from N2—CO—B2— | 73 | D | 0.05 | ESI: (M − H)⁻ = 815/ | | colourless |
| | | | | C1 with aq. 1M | | | | 817/819 (Br₂); | | crystals |
| | | | | LiOH, then aq. 1M | | | | (M + H)⁺ = 817/ | | |
| | | | | HCl | | | | 819/821 (Br₂); | | |
| | | | | | | | | (M + Na)⁺ = 839/ | | |
| | | | | | | | | 841/843 (Br₂) | | |
| 120 | N1 | B30 | C2 | from N1—CO—B30— | 40 | | | ESI: (M − H)⁻ = 785/ | | colourless |
| | | | | C1 with aq. 1M | | | | 787/789 (Br₂); | | amorphous |
| | | | | NaOH, then aq. | | | | (M + H)⁺ = 787/ | | substance |
| | | | | 1M HCl | | | | 789/791 (Br₂) | | |
| 121 | N1 | B30 | C4 | from N1—CO—B30— | 48 | | | ESI: (M − H)⁻ = 785/ | | colourless |
| | | | | C11 with aq. 1M | | | | 787/789 (Br₂); | | amorphous |
| | | | | NaOH, then aq. | | | | (M + H)⁺ = 787/ | | substance |
| | | | | 1M HCl | | | | 789/791 (Br₂) | | |
| 126 | N1 | B7 | C15 | from N1—CO—B7— | 77 | C | 0.00 | ESI: (M + H)⁺ = 773/ | | colourless |
| | | | | C14 with aq. 1M | | | | 775/777 (Br₂) | | solid |
| | | | | LiOH, then aq. 1M | | | | | | substance |
| | | | | HCl | | | | | | |
| 127 | N1 | B8 | C15 | from N1—CO—B8— | 100 | C | 0.00 | ESI: (M + H)⁺ = 651/ | | colourless |
| | | | | C14 with aq. 1M | | | | 657 (Cl) | | solid |
| | | | | LiOH, then aq. 1M | | | | | | substance |
| | | | | HCl | | | | | | |
| 134 | N1 | B30 | C47 | from N1—CO—B30— | 68 | KK | 0.25 | ESI: (M + H)⁺ = 787/ | | colourless |
| | | | | C45 with aq. 1M | | | | 789/791 (Br₂) | | solid |
| | | | | LiOH, then aq. 1M | | | | | | substance |
| | | | | HCl | | | | | | |
| 135 | N1 | B30 | C48 | from N1—CO—B30— | 29 | KK | 0.14 | ESI: (M + H)⁺ = 787/ | | colourless |
| | | | | C44 with aq. 1M | | | | 789/791 (Br₂) | | solid |
| | | | | LiOH, then aq. 1M | | | | | | substance |
| | | | | HCl | | | | | | |
| 136 | N1 | B30 | C49 | from N1—CO—B30— | 78 | KK | 0.10 | ESI: (M − H)⁻ = 785/ | | colourless |
| | | | | C46 with aq. 1M | | | | 787/789 (Br₂) | | solid |
| | | | | LiOH, then aq. 1M | | | | | | substance |
| | | | | HCl | | | | | | |
| 137 | N1 | B21 | C47 | from N1—CO—B21— | 81 | KK | 0.24 | ESI: (M + H)⁺ = 801/ | | colourless |
| | | | | C45 with aq. 1M | | | | 803/805 (Br₂) | | solid |
| | | | | LiOH, then aq. 1M | | | | | | substance |
| | | | | HCl | | | | | | |
| 139 | N1 | B21 | C48 | from N1—CO—B21— | 51 | KK | 0.11 | ESI: (M + H)⁺ = 801/ | | colourless |
| | | | | C44 with aq. 1M | | | | 803/805 (Br₂) | | solid |
| | | | | LiOH, then aq. 1M | | | | | | substance |
| | | | | HCl | | | | | | |
| 143 | N1 | B31 | C48 | from N1—CO—B31— | 74 | KK | 0.11 | ESI: (M + H)⁺ = 802/ | | colourless |
| | | | | C44 with aq. 1M | | | | 804/806 (Br₂) | | solid |
| | | | | LiOH, then aq. 1M | | | | | | substance |
| | | | | HCl | | | | | | |
| 145 | N1 | B31 | C47 | from N1—CO—B31— | 72 | KK | 0.23 | ESI: (M + H)⁺ = 802/ | | colourless |
| | | | | C45 with aq. 1M | | | | 804/806 (Br₂) | | solid |
| | | | | LiOH, then aq. 1M | | | | | | substance |
| | | | | HCl | | | | | | |
| 146 | N1 | B31 | C49 | from N1—CO—B31— | 62 | KK | 0.07 | ESI: (M + H)⁺ = 802/ | | colourless |
| | | | | C46 with aq. 1M | | | | 804/806 (Br₂) | | solid |
| | | | | LiOH, then aq. 1M | | | | | | substance |
| | | | | HCl | | | | | | |
| 147 | N1 | B21 | C49 | from N1—CO—B21— | 92 | KK | 0.08 | ESI: (M + H)⁺ = 801/ | | colourless |
| | | | | C46 with aq. 1M | | | | 803/805 (Br₂) | | solid |
| | | | | LiOH, then aq. 1M | | | | | | substance |
| | | | | HCl | | | | | | |
| 150 | N1 | B32 | C47 | from N1—CO—B32— | 17 | KK | 0.14 | ESI: (M + H)⁺ = 675 | | colourless |
| | | | | C45 with aq. 1M | | | | | | solid |
| | | | | LiOH, then aq. 1M | | | | | | substance |
| | | | | HCl | | | | | | |
| 157 | N1 | B21 | C51 | from N1—CO—B21— | 75 | Q | 0.35 | ESI: (M + H)⁺ = 801/ | | colourless |
| | | | | C50 with aq. 1M | | | | 803/805 (Br₂) | | amorphous |
| | | | | LiOH, then aq. 1M | | | | | | substance |
| | | | | HCl | | | | | | |
| 158 | N1 | B32 | C51 | from N1—CO—B32— | 20 | KK | 0.13 | ESI: (M − H)⁻ = 673; | | colourless |
| | | | | C50 with aq. 1M | | | | (M + H)⁺ = 675 | | amorphous |
| | | | | LiOH, then aq. 1M | | | | | | substance |
| | | | | HCl | | | | | | |
| 159 | N1 | B31 | C51 | from N1—CO—B31— | 91 | OO | 0.60 | ESI: (M + H)⁺ = 802/ | | colourless |
| | | | | C50 with aq. 1M | | | | 804/806 (Br₂) | | amorphous |
| | | | | LiOH, then aq. 1M | | | | | | substance |
| | | | | HCl | | | | | | |
| 160 | N1 | B25 | C51 | from N1—CO—B25— | 82 | Q | 0.25 | ESI: (M + H)⁺ = 777/ | | colourless |
| | | | | C50 with aq. 1M | | | | 779/781/783 | | amorphous |
| | | | | LiOH, then aq. 1M | | | | (BrCl₂) | | substance |
| | | | | HCl | | | | | | |
| 161 | N1 | B30 | C51 | from N1—CO—B30— | 73 | Q | 0.32 | ESI: (M + H)⁺ = 787/ | | colourless |
| | | | | C50 with aq. 1M | | | | 789/791 (Br₂) | | amorphous |
| | | | | LiOH, then aq. 1M | | | | | | substance |
| | | | | HCl | | | | | | |
| 163 | N1 | B25 | C47 | from N1—CO—B25— | 90 | KK | 0.17 | ESI: (M + H)⁺ = 777/ | | colourless |
| | | | | C45 with aq. 1M | | | | 779/781/783 | | amorphous |
| | | | | LiOH, then aq. 1M | | | | (BrCl₂) | | substance |
| | | | | HCl | | | | | | |
| 165 | N1 | B33 | C6 | from N1—CO—B33— | 78 | KK | 0.16 | ESI: (M + H)⁺ = 787/ | | colourless |
| | | | | C5 with aq. 1M | | | | 789/791 (Br₂) | | solid |
| | | | | LiOH, then aq. 1M | | | | | | substance |
| | | | | HCl | | | | | | |

### EXAMPLE 3

Ethyl 4-{1-[3-(1-naphthyl)-N-[[4-(2-oxo-1,3,4,5-tetrahydro-1,3-benzodiazepin-3-yl)-1-piperidinyl]carbonyl]-D-alanyl]-4-piperidinyl}-1-piperazineacetate (Ser. no. 81)

A tetrahydrofuran solution (20 ml) of 380.0 mg (0.84 mmol) ethyl 4-{1-[3-(1-naphthyl)-D-alanyl]-4-piperidinyl}-1-piperazineacetate was added dropwise over a period of 40 minutes to a stirred suspension of 149.356 mg (0.91 mmol) CDT in 10 ml of tetrahydrofuran cooled to −5° C. The reaction mixture was then stirred for 1 hour at −5° C. and 1 hour at ambient temperature and combined with the suspension of 206.075 mg (0.84 mmol) 3-(4-piperidinyl)-1,3,4,5-tetrahydro-1,3-benzodiazepin-2-one in 10 ml DMF. In order to obtain a homogeneous mixture, the tetrahydrofuran was distilled off at normal pressure, another 15 ml of DMF were added and the mixture was then heated to 100° C. for 2 hours. The reaction mixture was evaporated down in vacuo, the residue was purified by column chromatography using a gradient method developed in-house using mixtures of dichloromethane, methanol and conc. ammonia on silica gel, the appropriate fractions were triturated with ether and the solid obtained (450.0 mg; 74% of theory) was suction filtered and dried.

ESI-MS: (M+H)⁺=724

### EXAMPLE 4

(R,S)-4-{1-[2-[(4-amino-3,5-dibromophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid (Ser. no. 99)

This and the following syntheses were carried out using the Chemspeed ASW2000 synthesising robot (Chemspeed Ltd., Rheinstraβe 32, CH-4302 Augst, Switzerland).

Mixture:

AGV 1: 118.862 mg (0.200 mmol) of (R,S)-2-[(4-amino-3,5-dibromophenyl)methyl]-4-[4-(3,4-dihydro-2(1H)-oxoquinazolin-3-yl)-1-piperidinyl]-4-oxobutanoic acid in 3 ml THF;

AGV 2: 51.073 mg (0.200 mmol) of ethyl 4-(4-piperidinyl)-1-piperazineacetate in 2 ml THF;

AGV 3: 64.220 mg (0.200 mmol) of TBTU in 2 ml DMF;

AGV 4: 1.00 ml (1.00 mmol) of triethylamine;

AGV 5: 1.00 ml 4M sodium hydroxide solution;

AGV 6: 1.00 ml 4M hydrochloric acid;

AGV 7: 6 ml THF.

AGV 1 to 4 were positioned accordingly, then pipetted together by the robot and shaken for 8 hours at room temperature. The reaction mixtures were concentrated by evaporation, each combined with 7 ml of ethyl acetate, the solutions formed were each washed with 10 ml 10% aqueous potassium carbonate solution and with 6 ml of water and again freed from solvent. The residues were each dissolved in AGV 7 and after the addition of AGV 5 stirred for six hours at room temperature. The reaction mixtures were neutralised by the addition of AGV 6, then concentrated by evaporation. The residues obtained were each dissolved in 1.9 ml DMF and placed on a microtitre plate. The samples were in each case separated using an HPLC-MS apparatus (Agilent Technologies, Agilent 1100 Series Modules and Systems for HPLC and LC/MS), the products of interest were collected under mass control. The end products were freeze-dried.:

Yield: 26.0 mg (15% of theory).

ESI-MS: (M−H)⁻=800/802/804 (Br₂) (M+H)⁺=802/804/806 (Br₂)

The following compounds of general formula N-B-C were prepared analogously:

| Ser. | | | | | % | |
|---|---|---|---|---|---|---|
| no. | N | B | C | Remarks | yield | MS |
| 100 | N1 | B12 | C2 | coupling of N1—CO—B12—OH with | 8 | ESI: (M − H)⁻ = 803/805/807 |
| | | | | H—C1 and subsequent | | (Br₂); (M + H)⁺ = 805/807/809 |
| | | | | saponification with aq. NaOH | | (Br₂) |
| 101 | N5 | B13 | C2 | coupling of N5—CO—B13—OH with | 6 | ESI: (M + H)⁺ = 682 |
| | | | | H—C1 and subsequent | | |
| | | | | saponification with aq. NaOH | | |
| 102 | N1 | B14 | C2 | coupling of N1—CO—B14—OH with | 6 | ESI: (M + H)⁺ = 767 |
| | | | | H—C1 and subsequent | | |
| | | | | saponification with aq. NaOH | | |
| 103 | N1 | B15 | C2 | coupling of N1—CO—B15—OH with | 6 | ESI: (M + H)⁺ = 673 |
| | | | | H—C1 and subsequent | | |
| | | | | saponification with aq. NaOH | | |
| 104 | N1 | B16 | C2 | coupling of N1—CO—B16—OH with | 6 | ESI: (M − H)⁻ = 735/737 (Br); |
| | | | | H—C1 and subsequent | | (M + H)⁺ = 737/739 (Br) |
| | | | | saponification with aq. NaOH | | |
| 105 | N1 | B17 | C2 | coupling of N1—CO—B17—OH with | 10 | ESI: (M + H)⁺ = 699 |
| | | | | H—C1 and subsequent | | |
| | | | | saponification with aq. NaOH | | |
| 106 | N1 | B18 | C2 | coupling of N1—CO—B18—OH with | 4 | ESI: (M + H)⁺ = 689 |
| | | | | H—C1 and subsequent | | |
| | | | | saponification with aq. NaOH | | |
| 107 | N1 | B19 | C2 | coupling of N1—CO—B19—OH with | 4 | ESI: (M − H)⁻ = 712/714/716 |
| | | | | H—C1 and subsequent | | (Cl₂); (M + H)⁺ = 714/716/718 |
| | | | | saponification with aq. NaOH | | (Cl₂) |
| 108 | N1 | B20 | C2 | coupling of N1—CO—B20—OH with | 4 | ESI: (M + H)⁺ = 767 |
| | | | | H—C1 and subsequent | | |
| | | | | saponification with aq. NaOH | | |
| 109 | N1 | B21 | C2 | coupling of N1—CO—B21—OH with | 13 | ESI: (M − H)⁻ = 799/801/803 |
| | | | | H—C1 and subsequent | | (Br₂); (M + H)⁺ = 801/803/805 |
| | | | | saponification with aq. NaOH | | (Br₂) |
| 110 | N1 | B22 | C2 | coupling of N1—CO—B22—OH with | 4 | ESI: (M + H)⁺ = 865/ |
| | | | | H—C1 and subsequent | | 867/869/871 (Br₃) |
| | | | | saponification with aq. NaOH | | |
| 111 | N1 | B23 | C2 | coupling of N1—CO—B23—OH with | 12 | ESI: (M + H)⁺ = 691 |
| | | | | H—C1 and subsequent | | |
| | | | | saponification with aq. NaOH | | |
| 112 | N1 | B24 | C2 | coupling of N1—CO—B24—OH with | 2 | ESI: (M + H)⁺ = 699/701/703 |
| | | | | H—C1 and subsequent | | (Cl₂) |
| | | | | saponification with aq. NaOH | | |
| 113 | N1 | B25 | C2 | coupling of N1—CO—B25—OH with | 4 | ESI: (M + H)⁺ = 777/779/781 |
| | | | | H—C1 and subsequent | | (Br, Cl₂) |
| | | | | saponification with aq. NaOH | | |
| 114 | N1 | B26 | C2 | coupling of N1—CO—B26—OH with | 3 | ESI: (M + H)⁺ = 681 |
| | | | | H—C1 and subsequent | | |
| | | | | saponification with aq. NaOH | | |
| 115 | N1 | B27 | C2 | coupling of N1—CO—B27—OH with | 4 | ESI: (M − H)⁻ = 671; (M + H)⁺ = 673 |
| | | | | H—C1 and subsequent | | |
| | | | | saponification with aq. NaOH | | |
| 116 | N1 | B28 | C2 | coupling of N1—CO—B28—OH with | 4 | ESI: (M + H)⁺ = 685 |
| | | | | H—C1 and subsequent | | |
| | | | | saponification with aq. NaOH | | |
| 117 | N6 | B21 | C2 | coupling of N6—CO—B21—OH with | 3 | ESI: (M + H)⁺ = 837/839/841 |
| | | | | H—C1 and subsequent | | (Br₂) |
| | | | | saponification with aq. NaOH | | |
| 118 | N1 | B29 | C2 | coupling of N1—CO—B29—OH with | 4 | ESI: (M + H)⁺ = 699/701/703 |
| | | | | H—C1 and subsequent | | (Cl₂) |
| | | | | saponification with aq. NaOH | | |

The Examples that follow describe the preparation of pharmaceutical formulations which contain as active substance any desired compound of general formula (1):

### EXAMPLE I

Capsules for Powder Inhalation Containing 1 mg of Active Ingredient

Composition:

1 capsule for powder inhalationcontains:

| | |
|---|---|
| active ingredient | 1.0 mg |
| lactose | 20.0 mg |
| hard gelatine capsules | 50.0 mg |
| | 71.0 mg |

Method of Preparation:

The active ingredient is ground to the particle size required for inhaled substances. The ground active ingredient is homogeneously mixed with the lactose. The mixture is transferred into hard gelatine capsules.

### EXAMPLE II

Inhalable Solution for Respimat® Containing 1 mg of Active Ingredient

Composition:

1 puff contains:

| | |
|---|---|
| active ingredient | 1.0 mg |
| benzalkonium chloride | 0.002 mg |
| disodium edetate | 0.0075 mg |
| purified water ad | 15.0 μl |

Method of Preparation:

The active ingredient and benzalkonium chloride are dissolved in water and transferred into Respimat® cartridges.

### EXAMPLE III

Inhalable Solution for Nebulisers Containing 1 mg of Active Ingredient

Composition:

1 vial contains:

| | |
|---|---|
| active ingredient | 0.1 g |
| sodium chloride | 0.18 g |
| benzalkonium chloride | 0.002 g |
| purified water ad | 20.0 ml |

Method of Preparation

The active ingredient, sodium chloride and benzalkonium chloride are dissolved in water.

### EXAMPLE IV

Propellant Gas-Operated Metering Aerosol Containing 1 mg of Active Ingredient

Composition:

1 puff contains:

| | |
|---|---|
| active ingredient | 1.0 mg |
| lecithin | 0.1% |
| propellant gas ad | 50.0 μl |

Method of Preparation:

The micronised active ingredient is homogeneously suspended in the mixture of lecithin and propellant gas. The suspension is transferred into a pressurised container with a metering valve.

### EXAMPLE V

Nasal Spray Containing 1 mg of Active Ingredient

Composition:

| | |
|---|---|
| active ingredient | 1.0 mg |
| sodium chloride | 0.9 mg |
| benzalkonium chloride | 0.025 mg |
| disodium edetate | 0.05 mg |
| purified water ad | 0.1 ml |

Method of Preparation:

The active ingredient and the excipients are dissolved in water and transferred into a suitable container.

### EXAMPLE VI

Injectable Solution Containing 5 mg of Active Substance Per 5 ml

Composition:

| | |
|---|---|
| active substance | 5 mg |
| glucose | 250 mg |
| human serum albumin | 10 mg |
| glycofurol | 250 mg |
| water for injections ad | 5 ml |

Preparation:

Glycofurol and glucose are dissolved in water for injections (Wfl); human serum albumin is added; active ingredient is dissolved with heating; made up to specified volume with Wfl; transferred into ampoules under nitrogen gas.

### EXAMPLE VII

Injectable Solution Containing 100 mg of Active Substance Per 20 ml

Composition:

| | |
|---|---|
| active substance | 100 mg |
| monopotassium dihydrogen phosphate = KH₂PO₄ | 12 mg |
| disodium hydrogen phosphate = Na₂HPO₄.2H₂O | 2 mg |
| sodium chloride | 180 mg |
| human serum albumin | 50 mg |
| Polysorbate 80 | 20 mg |
| water for injections ad | 20 ml |

Preparation:

Polysorbate 80, sodium chloride, monopotassium dihydrogen phosphate and disodium hydrogen phosphate are dissolved in water for injections (Wfl); human serum albumin is added; active ingredient is dissolved with heating; made up to specified volume with Wfl; transferred into ampoules.

### EXAMPLE VIII

Lyophilisate Containing 10 mg of Active Substance

Composition:

| | |
|---|---|
| Active substance | 10 mg |
| Mannitol | 300 mg |
| human serum albumin | 20 mg |

Preparation:

Mannitol is dissolved in water for injections (Wfl); human serum albumin is added; active ingredient is dissolved with heating; made up to specified volume with Wfl; transferred into vials; freeze-dried.

Solvent for Lyophilisate:

| | |
|---|---|
| Polysorbate 80 = Tween 80 | 20 mg |
| mannitol | 200 mg |
| water for injections ad | 10 ml |

Preparation:

Polysorbate 80 and mannitol are dissolved in water for injections (Wfl); transferred into ampoules.

### EXAMPLE IX

Tablets Containing 20 mg of Active Substance

Composition:

| | |
|---|---|
| active substance | 20 mg |
| lactose | 120 mg |
| maize starch | 40 mg |
| magnesium stearate | 2 mg |
| Povidone K 25 | 18 mg |

Preparation:

Active substance, lactose and maize starch are homogeneously mixed; granulated with an aqueous solution of Povidone; mixed with magnesium stearate; compressed in a tablet press; weight of tablet 200 mg.

### EXAMPLE X

Capsules Containing 20 mg Active Substance

Composition:

| | |
|---|---|
| active substance | 20 mg |
| maize starch | 80 mg |
| highly dispersed silica | 5 mg |
| magnesium stearate | 2.5 mg |

Preparation:

Active substance, maize starch and silica are homogeneously mixed; mixed with magnesium stearate; the mixture is packed into size for 3 hard gelatine capsules in a capsule filling machine.

### EXAMPLE XI

Suppositories Containing 50 mg of Active Substance

Composition:

| | |
|---|---|
| active substance | 50 mg |
| hard fat (Adeps solidus) q.s. ad | 1700 mg |

Preparation:

Hard fat is melted at about 38° C.; ground active substance is homogeneously dispersed in the molten hard fat; after cooling to about 35° C. it is poured into chilled moulds.

### EXAMPLE XII

Injectable Solution Containing 10 mg of Active Substance Per 1 ml

Composition:

| | |
|---|---|
| active substance | 10 mg |
| mannitol | 50 mg |
| human serum albumin | 10 mg |
| water for injections ad | 1 ml |

Preparation:

Mannitol is dissolved in water for injections (Wfl); human serum albumin is added; active ingredient is dissolved with heating; made up to specified volume with Wfl; transferred into ampoules under nitrogen gas.

What is claimed is:

1. Carboxylic acids and esters of general formula 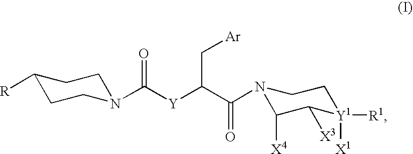
wherein
R denotes a monounsaturated 5- to 7-membered diaza, triaza or S,S-dioxido-thiadiaza heterocycle,
while the above-mentioned heterocycles are linked via a nitrogen atom and are characterised by a carbonyl group or sulphonyl group each flanked by two nitrogen atoms,
may be substituted at one or at two carbon atoms by an alkyl, phenyl, pyridinyl, thienyl or 1,3-thiazolyl group, while the substituents may be identical or different,
and the double bond of one of the above-mentioned unsaturated heterocycles may be fused to a benzene, pyridine or quinoline ring,
while the phenyl, pyridinyl, thienyl, or 1,3-thiazolyl groups contained in R as well as benzo-, pyrido- and quinolino-fused heterocycles in the carbon skeleton may additionally be mono-, di- or trisubstituted by fluorine, chlorine or bromine atoms, by alkyl, alkoxy, nitro, alkylthio, alkylsulphinyl, alkylsulphonyl, alkylsulphonylamino, phenyl, trifluoromethyl, alkoxycarbonyl, carboxy, dialkylamino, hydroxy, amino, acetylamino, propionylamino, aminocarbonyl, alkylaminocarbonyl, dialkylaminocarbonyl, methylenedioxy, aminocarbonylamino, alkanoyl, cyano, trifluoromethoxy, trifluoromethylthio, trifluoromethylsulphinyl or trifluoromethylsulphonyl groups, while the substituents may be identical or different,
Ar denotes a phenyl, 1-naphthyl, 2-naphthyl, tetrahydro-1-naphthyl, tetrahydro-2-naphthyl, 1H-indol-3-yl, 1-methyl-1H-indol-3-yl, 1-formyl-1H-indol-3-yl, 4-imidazolyl, 1-methyl-4-imidazolyl, 2-thienyl, 3-thienyl, thiazolyl, 1H-indazol-3-yl, 1-methyl-1H-indazol-3-yl, benzo[b]furyl, 2,3-dihydrobenzo[b]furyl, benzo[b]thienyl, pyridinyl, quinolinyl or isoquinolinyl group,
while the above-mentioned aromatic and heteroaromatic groups may additionally be mono-, di- or trisubstituted in the carbon skeleton by fluorine, chlorine or bromine atoms, by alkyl groups, C₃₋₈-cycloalkyl groups, phenylalkyl groups, alkenyl, alkoxy, phenyl, phenylalkoxy, trifluoromethyl, alkoxycarbonyl, carboxy, dialkylamino, nitro, hydroxy, amino, alkylamino, acetylamino, propionylamino, methylsulphonyloxy, aminocarbonyl, alkylaminocarbonyl, dialkylaminocarbonyl, alkanoyl, cyano, trifluoromethoxy, trifluoromethylthio, trifluoromethyl-sulphinyl or trifluoromethylsulphonyl groups and the substituents may be identical or different,
Y denotes the methylene or the —NH— group,
Y¹ denotes the carbon or the nitrogen atom,
X¹ denotes the pair of free electrons, if Y¹ denotes the nitrogen atom, or, if Y¹ is the carbon atom, denotes a hydrogen atom or a carboxylic acid group optionally esterified with a lower aliphatic alcohol,
X³ and X⁴ in each case denote the hydrogen atom or the carboxylic acid group optionally esterified with a lower aliphatic alcohol,
with the proviso that at least one but also not more than one of the groups X¹, X², X³ or X⁴ contains an optionally esterified carboxylic acid function, and
R¹ denotes a group of general formula 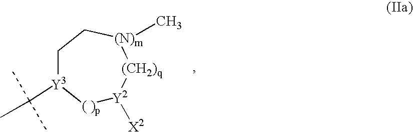
wherein
Y² denotes the carbon or, if m assumes the value 0, also the nitrogen atom,
Y³, which is always different from Y¹, denotes the carbon or nitrogen atom, X² denotes a group of general formula
CH₂CO₂R², (III)
wherein
R² denotes the hydrogen atom or a C₁₋₅-alkyl group,
or, if Y² is the carbon atom, it may also denote the hydrogen atom or the carboxylic acid group optionally esterified with a lower aliphatic alcohol,
m denotes the numbers 0 or 1,
p denotes the numbers 0, 1, 2 or 3 and
q denotes the numbers 0, 1 or 2,
while the sum of m, p and q may assume the values 1, 2 or 3,
or one of the groups (IIb), (IIc) or (IId) 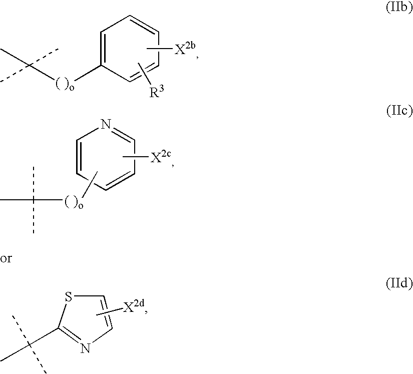
wherein
X^{2b}, X^{2c} and X^{2d} each denote the hydrogen atom or a carboxylic acid group optionally esterified with a lower aliphatic alcohol,
o denotes the numbers 0, 1, 2 or 3 and
R³ denotes the hydrogen atom, the fluorine, chlorine or bromine atom, an alkyl, alkoxy, nitro, trifluoromethyl, hydroxy, amino, acetylamino, aminocarbonyl, acetyl or cyano group,
while, unless otherwise stated, the above-mentioned alkyl groups or the alkyl groups contained in the above-mentioned groups contain 1 to 5 carbon atoms and may be straight-chain or branched,
or the tautomers or the diastereomers or the enantiomers or the mixtures thereof or the salts thereof.

2. Carboxylic acids and esters of general formula I according to claim 1, wherein
R denotes a monounsaturated 5- to 7-membered diaza, triaza or S,S-dioxido-thiadiaza heterocycle,
while the above-mentioned heterocycles are linked via a nitrogen atom and
are characterised by a carbonyl group or sulphonyl group in each case flanked by two nitrogen atoms,
may be substituted at a carbon atom by a phenyl, pyridinyl, thienyl or 1,3-thiazolyl group,
and the double bond of one of the above-mentioned unsaturated heterocycles may be fused to a benzene, pyridine or quinoline ring,
while the phenyl, pyridinyl, thienyl, or 1,3-thiazolyl groups contained in R as well as benzo-, pyrido- and quinolino-fused heterocycles in the carbon skeleton may additionally be mono-, di- or trisubstituted by fluorine, chlorine or bromine atoms, by alkyl, alkoxy, trifluoromethyl, amino, cyano or acetylamino groups, while the substituents may be identical or different,
Ar denotes a phenyl, 1-naphthyl, 2-naphthyl, 1,2,3,4-tetrahydro-1-naphthyl or 2,3-dihydrobenzo[b]fur-5-yl group,
while the above-mentioned aromatic and heteroaromatic groups may additionally be mono-, di- or trisubstituted in the carbon skeleton by fluorine, chlorine or bromine atoms, by alkyl groups, alkoxy, trifluoromethyl, nitro, hydroxy, amino, aminocarbonyl, acetyl or cyano groups and the substituents may be identical or different,
Y denotes the methylene or the —NH— group,
Y¹ denotes the carbon or the nitrogen atom,
X¹ denotes a pair of free electrons, if Y¹ denotes the nitrogen atom, or, if Y¹ is the carbon atom, the hydrogen atom or the carboxylic acid group optionally esterified with a lower aliphatic alcohol,
X³ and X⁴ each denote the hydrogen atom or the carboxylic acid group optionally esterified with a lower aliphatic alcohol,
with the proviso that at least one but also not more than one of the groups X¹, X², X³ or X⁴ contains an optionally esterified carboxylic acid function, and
R¹ denotes a group of general formula 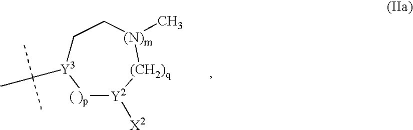
wherein
Y² denotes the carbon atom or, if m assumes the value 0, may also denote the nitrogen atom,
Y³, which is always different from Y¹, denotes the carbon or the nitrogen atom,
X² denotes a group of general formula
CH₂CO₂R², (III)
wherein
R² denotes the hydrogen atom or a C₁₋₅-alkyl group,
or, if Y² is the carbon atom, also denotes the hydrogen atom or the carboxylic acid group optionally esterified with a lower aliphatic alcohol,
m denotes the numbers 0 or 1,
p denotes the numbers 0, 1 or 2 and
q denotes the numbers 0, 1 or 2,
while the sum of m, p and q may assume the values 1 or 2,
or one of the groups 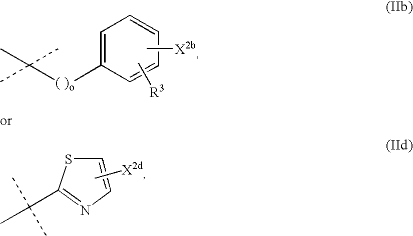
wherein
X^{2b} and X^{2d} each denote the hydrogen atom or the carboxylic acid group optionally esterified with a lower aliphatic alcohol,
o denotes the numbers 0, 1, 2 or 3 and
R³ denotes the hydrogen atom, the fluorine, chlorine or bromine atom, a methyl, methoxy, nitro, trifluoromethyl or cyano group,
while, unless otherwise stated, the above-mentioned alkyl groups or the alkyl groups contained in the above-mentioned groups contain 1 to 4 carbon atoms and may be branched or unbranched,
or the tautomers or the diastereomers or the enantiomers or the salts thereof.

3. Carboxylic acids and esters of general formula I according to claim 1, wherein
R denotes a monounsaturated 5- to 7-membered diaza, triaza or S,S-dioxido-thiadiaza heterocycle,
while the above-mentioned heterocycles are linked via a nitrogen atom and
are characterised by a carbonyl group or sulphonyl group each flanked by two nitrogen atoms,
may be substituted at a carbon atom by a phenyl group,
and the double bond of one of the above-mentioned unsaturated heterocycles may be fused to a benzene, pyridine or quinoline ring,
while the phenyl groups contained in R as well as benzo-, pyrido- and quinolino-fused heterocycles may additionally be mono- or disubstituted in the carbon skeleton by fluorine, chlorine or bromine atoms, by methyl, methoxy, trifluoromethyl, or cyano groups, while the substituents may be identical or different,
Ar denotes a phenyl, 1-naphthyl, 2-naphthyl, 1,2,3,4-tetrahydro-1-naphthyl or 2,3-dihydrobenzo[b]fur-5-yl group,
while the above-mentioned aromatic and heteroaromatic groups may additionally be mono-, di- or trisubstituted in the carbon skeleton by fluorine, chlorine or bromine atoms, by methyl, methoxy, trifluoromethyl, hydroxy or amino groups and the substituents may be identical or different,
Y denotes the methylene or —NH— group,
Y¹ denotes the carbon or nitrogen atom,
X¹ denotes a pair of free electrons, if Y¹ denotes the nitrogen atom, or, if Y¹ is the carbon atom, the hydrogen atom or the carboxylic acid group optionally esterified with methanol or ethanol,
X³ and X⁴ each denote the hydrogen atom or the carboxylic acid group optionally esterified with methanol or ethanol,
with the proviso that at least one but also not more than one of the groups X¹, X², X³ or X⁴ contains an optionally esterified carboxylic acid function, and
R¹ denotes a group of general formula 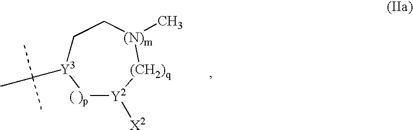
wherein
Y² denotes the carbon or, if m assumes the value 0, also denotes the nitrogen atom,
Y³, which is always different from Y¹, denotes the carbon or the nitrogen atom,
X² denotes a group of general formula
CH₂CO₂R², (III)
wherein
R² denotes the hydrogen atom or a straight-chain or branched C₁₋₄-alkyl group,
or, if Y² is the carbon atom, also denotes the hydrogen atom or the carboxylic acid group optionally esterified with methanol or ethanol,
m denotes the numbers 0 or 1,
p denotes the numbers 0, 1 or 2 and
q denotes the numbers 0, 1 or 2,
while the sum of m, p and q may assume the values 1 or 2,
or one of the groups 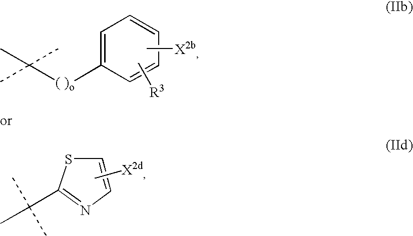
wherein
X^{2b} and X^{2d} each denote the hydrogen atom or the carboxylic acid group optionally esterified with methanol or ethanol,
o denotes the numbers 0, 1 or 2 and
R³ denotes the hydrogen atom, the fluorine, chlorine or bromine atom, a methyl, methoxy or trifluoromethyl group,
while, unless otherwise stated, the above-mentioned alkyl groups or the alkyl groups contained in the above-mentioned groups contain 1 to 4 carbon atoms and may be straight-chain or branched,
or the tautomers or the diastereomers or the enantiomers or the salts thereof.

4. Carboxylic acids and esters of general formula I according to claim 1, wherein R denotes the 3,4-dihydro-2(1H)-oxoquinazolin-3-yl, 2,4-dihydro-5-phenyl-3(3H)-oxo-1,2,4-triazol-2-yl, 1,3-dihydro-2(2H)-oxoimidazo[4,5-c]quinolin-3-yl, 2-oxo-1,3,4,5-tetrahydro-1,3-benzodiazepin-3-yl, 3,4-dihydro-2(1H)-oxopyrido[3,4-d]pyrimidin-3-yl or 3,4-dihydro-2,2-dioxido-2,1,3-benzothiadiazin-3-yl group,
Ar denotes the 3,5-dibromo-4-hydroxyphenyl, 4-amino-3,5-dibromophenyl, 4-bromo-3,5-dimethylphenyl, 3,5-dichloro-4-methylphenyl, 3,4-dibromophenyl, 3-bromo-4,5-dimethylphenyl, 3,5-dibromo-4-methylphenyl, 3-chloro-4-methylphenyl, 3,4-difluorophenyl, 4-hydroxyphenyl, 1-naphthyl, 3,5-dibromo-4-fluorophenyl, 3,5-bis-(trifluoromethyl)-phenyl, 3,4,5-trimethylphenyl, 3-(trifluoromethyl)-phenyl, 3,5-dimethyl-4-methoxyphenyl, 4-amino-3,5-dichlorophenyl, 2,4-bis-(trifluoromethyl)-phenyl, 3,4,5-tribromophenyl, 3,4-dimethoxyphenyl, 3,4-dichlorophenyl, 4-bromo-3,5-dichlorophenyl, 2-naphthyl, 2,3-dihydrobenzo[b]fur-5-yl, 1,2,3,4-tetrahydro-1-naphthyl or 2,3-dichlorophenyl group,
Y denotes the methylene or the —NH— group,
Y¹ denotes the carbon or the nitrogen atom,
X¹ denotes a pair of free electrons, if Y¹ denotes the nitrogen atom, or, if Y¹ is the carbon atom, the hydrogen atom, the carboxylic acid or the methoxycarbonyl group and
R¹ denotes a group of general formula 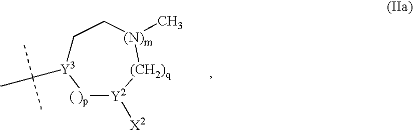
wherein
Y² denotes the carbon atom or, if m assumes the value 0, also the nitrogen atom,
Y³, which is always different from Y¹, denotes the carbon or the nitrogen atom,
X² denotes a group of general formula
CH₂CO₂R², (III)
wherein
R² denotes the hydrogen atom or a straight-chain or branched C₁₋₄-alkyl group,
or, if Y² is the carbon atom, also denotes the hydrogen atom or the carboxylic acid group optionally esterified with methanol or ethanol,
m denotes the numbers 0 or 1,
p and q in each case denotes the numbers 0, 1 or 2,
while the sum of m, p and q may assume the values 1 or 2,
or one of the groups 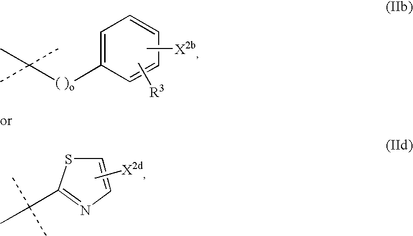
wherein
X^{2b} denotes the hydrogen atom or the carboxylic acid group optionally esterified with methanol or ethanol,
X^{2d} denotes the hydrogen atom or the carboxylic acid group optionally esterified with methanol,
o denotes the numbers 0, 1 or 2 and
R³ denotes the hydrogen atom or the trifluoromethyl group,
while, unless otherwise stated, the above-mentioned alkyl groups or the alkyl groups contained in the above-mentioned groups contain 1 to 4 carbon atoms and may be straight-chain or branched,
or the tautomers or the diastereomers or the enantiomers or the salts thereof.

5. The following carboxylic acids and esters of general formula I according to claim 1:
(1) ethyl 4-{1-[3,5-dibromo-N-[[4-(3,4-dihydro-2(1H)-oxoquinazolin-3-yl)-1-piperidinyl]carbonyl]-D-tyrosyl]-4-piperidinyl}-1-piperazineacetate,
(2) 4-{1-[3,5-dibromo-N-[[4-(3,4-dihydro-2(1H)-oxoquinazolin-3-yl)-1-piperidinyl]carbonyl]-D-tyrosyl]-4-piperidinyl}-1-piperazineacetic acid,
(3) 1,1-dimethylethyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-1-piperidineacetate,
(4) 4-{4-[3,5-dibromo-N-[[4-(3,4-dihydro-2(1H)-oxoquinazolin-3-yl)-1-piperidinyl]carbonyl]-D-tyrosyl]-1-piperazinyl}-1-piperidineacetic acid,
(5) methyl 1′-[3,5-dibromo-N-[[4-(3,4-dihydro-2(1H)-oxoquinazolin-3-yl)-1-piperidinyl]carbonyl]-D-tyrosyl]-[1,4′]bipiperidinyl-4-acetate,
(6) 1′-[3,5-dibromo-N-[[4-(3,4-dihydro-2(1H)-oxoquinazolin-3-yl)-1-piperidinyl]-carbonyl]-D-tyrosyl]-[1,4′]bipiperidinyl-4-acetic acid,
(7) ethyl endo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylate,
(8) endo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylic acid,
(9) ethyl exo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylate,
(10) exo-4-{4-[3,5-dibromo-N-[[4-(3,4-dihydro-2(1H)-oxoquinazolin-3-yl)-1-piperidinyl]carbonyl]-D-tyrosyl]-1-piperazinyl}-cyclohexanecarboxylic acid,
(11) ethyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-1-piperidineacetate,
(12) methyl 1′-[4-amino-3,5-dibromo-N-[[4-(2-oxo-1,3,4,5-tetrahydro-1,3-benzodiazepin-3-yl)-1-piperidinyl]carbonyl]-D-phenylalanyl]-[1,4′]bipiperidinyl-4-acetate,
(13) 1′-[4-amino-3,5-dibromo-N-[[4-(2-oxo-1,3,4,5-tetrahydro-1,3-benzodiazepin-3-yl)-1-piperidinyl]carbonyl]-D-phenylalanyl]-[1,4′] bipiperidinyl-4-acetic acid,
(14) ethyl 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-1-piperidineacetate,
(15) ethyl 4-{1-[4-bromo-N-[[4-(3,4-dihydro-2(1H)-oxoquinazolin-3-yl)-1-piperidinyl]carbonyl]-3,5-dimethyl-D,L-phenylalanyl]-4-piperidinyl}-1-piperazineacetate,
(16) ethyl 4-{1-[3,5-dichloro-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetate,
(17) ethyl 4-{1-[3,4-dibromo-N-[[4-(3,4-dihydro-2(1H)-oxoquinazolin-3-yl) 1-piperidinyl]carbonyl]-D,L-phenylalanyl]-4-piperidinyl}-1-piperazineacetate,
(18) ethyl 4-{1-[3-bromo-N-[[4-(3,4-dihydro-2(1H)-oxoquinazolin-3-yl)-1-piperidinyl]carbonyl]-4,5-dimethyl-D,L-phenylalanyl]-4-piperidinyl}-1-piperazineacetate,
(19) ethyl 4-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetate,
(20) ethyl 4-{1-[3-chloro-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetate,
(21) ethyl 4-{4-[4-bromo-N-[[4-(3,4-dihydro-2(1H)-oxoquinazolin-3-yl)-1-piperidinyl]carbonyl]-3,5-dimethyl-D,L-phenylalanyl]-1-piperazinyl}-1-piperidineacetate,
(22) 4-{1-[4-bromo-N-[[4-(3,4-dihydro-2(1H)-oxoquinazolin-3-yl)-1-piperidinyl]-carbonyl]-3,5-dimethyl-D,L-phenylalanyl]-4-piperidinyl}-1-piperazineacetic acid,
(23) 4-{1-[3,5-dichloro-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetic acid,
(24) 4-{1-[3,4-dibromo-N-[[4-(3,4-dihydro-2(1H)-oxoquinazolin-3-yl)-1-piperidinyl]carbonyl]-D,L-phenylalanyl]-4-piperidinyl}-1-piperazineacetic acid,
(25) 4-{1-[3-bromo-N-[[4-(3,4-dihydro-2(1H)-oxoquinazolin-3-yl)-1-piperidinyl]-carbonyl]-4,5-dimethyl-D,L-phenylalanyl]-4-piperidinyl}-1-piperazineacetic acid,
(26) 4-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-1-piperazineacetic acid,
(27) 4-{1-[3-chloro-N-[[4-(3,4-dihydro-2(1H)-oxoquinazolin-3-yl)-1-piperidinyl]-carbonyl]-4-methyl-D,L-phenylalanyl]-4-piperidinyl}-1-piperazineacetic acid,
(28) 4-{4-[4-bromo-N-[[4-(3,4-dihydro-2(1H)-oxoquinazolin-3-yl)-1-piperidinyl]-carbonyl]-3,5-dimethyl-D,L-phenylalanyl]-1-piperazinyl}-1-piperidineacetic acid,
(29) 1,1-dimethylethyl 4-{1-[3,4-difluoro-N-[[4-(3,4-dihydro-2(1H)-oxoquinazolin-3-yl)-1-piperidinyl]carbonyl]-D,L-phenylalanyl]-4-piperidinyl}-1-piperazineacetate,
(30) methyl 1′-[N-[[4-(3,4-dihydro-2(1H)-oxoquinazolin-3-yl)-1-piperidinyl]carbonyl]-D-tyrosyl]-[1,4′]bipiperidinyl-4-acetate,
(31) ethyl 4-{1-[N-[[4-(3,4-dihydro-2(1H)-oxoquinazolin-3-yl)-1-piperidinyl]carbonyl]-D-tyrosyl]-4-piperidinyl}-1-piperazineacetate,
(32) ethyl(R,S)-4-{1-[2-[(3,5-dibromo-4-methyl phenyl)methyl]-4-[4-(3,4-dihydro-2(1H)-oxoquinazolin-3-yl)-1-piperidinyl]-1,4-dioxobutyl]-4-piperidinyl}-1-piperazineacetate,
(33) methyl 1-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-(S)-pyrrolidine-2-carboxylate,
(34) methyl 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(S)-pyrrolidine-2-carboxylate,
(35) 1-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-(S)-pyrrolidine-2-carboxylic acid,
(36) 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(S)-pyrrolidine-2-carboxylic acid,
(37) methyl 1-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-(R)-pyrroidine-2-carboxylate,
(38) methyl 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(R)-pyrrolidine-2-carboxylate,
(39) 1-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-(R)-pyrrolidine-2-carboxylic acid,
(40) 1-{1-[3,5-dibromo-N-[[4-(3,4-dihydro-2(1H)-oxoquinazolin-3-yl)-1-piperidinyl]carbonyl]-D-tyrosyl]-4-piperidinyl}-(R)-pyrrolidine-2-carboxylic acid,
(41) methyl 1′-[4-amino-3,5-dibromo-N-[[4-(3,4-dihydro-2(1H)-oxoquinazolin-3-yl)-1-piperidinyl]carbonyl]-D-phenylalanyl]-(R)-[1,4′]bipiperidinyl-2-carboxylate,
(42) methyl 1′-[3,5-dibromo-N-[[4-(3,4-dihydro-2(1H)-oxoquinazolin-3-yl)-1-piperidinyl]carbonyl]-D-tyrosyl]-(R)-[1,4′]bipiperidinyl-2-carboxylate,
(43) methyl 1′-[4-amino-3,5-dibromo-N-[[4-(3,4-dihydro-2(1H)-oxoquinazolin-3-yl)-1-piperidinyl]carbonyl]-D-phenylalanyl]-(S)-[1,4′]bipiperidinyl-2-carboxylate,
(44) methyl 1′-[3,5-dibromo-N-[[4-(3,4-dihydro-2(1H)-oxoquinazolin-3-yl)-1-piperidinyl]carbonyl]-D-tyrosyl]-(S)-[1,4′]bipiperidinyl-2-carboxylate,
(45) 1′-[4-amino-3,5-dibromo-N-[[4-(3,4-dihydro-2(1H)-oxoquinazolin-3-yl)-1-piperidinyl]carbonyl]-D-phenylalanyl]-(R)-[1,4′]bipiperidinyl-2-carboxylic acid,
(46) 1′-[3,5-dibromo-N-[[4-(3,4-dihydro-2(1H)-oxoquinazolin-3-yl)-1-piperidinyl]-carbonyl]-D-tyrosyl]-(R)-[1,4′]bipiperidinyl-2-carboxylic acid,
(47) 1′-[3,5-dibromo-N-[[4-(3,4-dihydro-2(1H)-oxoquinazolin-3-yl)-1-piperidinyl]-carbonyl]-D-tyrosyl]-(S)-[1,4′]bipiperidinyl-2-carboxylic acid,
(48) 1′-[4-amino-3,5-dibromo-N-[[4-(3,4-dihydro-2(1H)-oxoquinazolin-3-yl)-1-piperidinyl]carbonyl]-D-phenylalanyl]-(S)-[1,4′]bipiperidinyl-2-carboxylic acid,
(49) methyl 1′-[3,5-dibromo-N-[[4-(3,4-dihydro-2(1H)-oxoquinazolin-3-yl)-1-piperidinyl]carbonyl]-D-tyrosyl]-[1,4′]bipiperidinyl-4′-carboxylate,
(50) methyl 1′-[4-amino-3,5-dibromo-N-[[4-(3,4-dihydro-2(1H)-oxoquinazolin-3-yl)-1-piperidinyl]carbonyl]-D-phenylalanyl]-[1,4′]bipiperidinyl-4′-carboxylate,
(51) 1′-[3,5-dibromo-N-[[4-(3,4-dihydro-2(1H)-oxoquinazolin-3-yl)-1-piperidinyl]-carbonyl]-D-tyrosyl]-[1,4′]bipiperidinyl-4′-carboxylic acid,
(52) 1′-[4-amino-3,5-dibromo-N-[[4-(3,4-dihydro-2(1H)-oxoquinazolin-3-yl)-1-piperidinyl]carbonyl]-D-phenylalanyl]-[1,4′]bipiperidinyl-4′-carboxylic acid,
(53) 1′-[N-[[4-(3,4-dihydro-2(1H)-oxoquinazolin-3-yl)-1-piperidinyl]carbonyl]-D-tyrosyl]-[1,4′]bipiperidinyl-4-acetic acid,
(54) 4-{1-[N-[[4-(3,4-dihydro-2(1H)-oxoquinazolin-3-yl)-1-piperidinyl]carbonyl]-D-tyrosyl]-4-piperidinyl}-1-piperazineacetic acid,
(55) ethyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoate,
(56) ethyl 3-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoate,
(57) methyl 4-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoate,
(58) ethyl 4-{1-[3,5-dibromo-N-[(4-(3,4-dihydro-2(1H)-oxoquinazolin-3-yl)-1-piperidinyl]carbonyl]-D-tyrosyl]-4-piperidinylmethyl}-benzoate,
(59) ethyl 4-{2-[1-[3,5-dibromo-N-[[4-(3,4-dihydro-2(1H)-oxoquinazolin-3-yl)-1-piperidinyl]carbonyl]-D-tyrosyl]-4-piperidinyl]-ethyl}-benzoate,
(60) methyl 4-{4-[3,5-dibromo-N-[[4-(3,4-dihydro-2(1H)-oxoquinazolin-3-yl)-1-piperidinyl]carbonyl]-D-tyrosyl]-1-piperazinyl}-3-(trifluoromethyl)-benzoate,
(61) methyl 3-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoate,
(62) ethyl 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoate,
(63) ethyl 3-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoate,
(64) methyl 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-benzoate,
(65) methyl 4-{2-[1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl]-ethyl}-benzoate,
(66) methyl 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-3-(trifluoromethyl)-benzoate,
(67) methyl 3-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-benzoate,
(68) 4-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoic acid,
(69) 3-{4-[3,5-dibromo-N-[[4-(3,4-dihydro-2(1H)-oxoquinazolin-3-yl)-1-piperidinyl]carbonyl]-D-tyrosyl]-1-piperazinyl}-benzoic acid,
(70) 4-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoic acid,
(71) 4-{1-[3,5-dibromo-N-[[4-(3,4-dihydro-2(1H)-oxoquinazolin-3-yl)-1-piperidinyl]carbonyl]-D-tyrosyl]-4-piperidinylmethyl}-benzoic acid,
(72) 4-{2-[1-[3,5-dibromo-N-[[4-(3,4-dihydro-2(1H)-oxoquinazolin-3-yl)-1-piperidinyl]carbonyl]-D-tyrosyl]-4-piperidinyl]-ethyl}-benzoic acid,
(73) 4-{4-[3,5-dibromo-N-[[4-(3,4-dihydro-2(1%)-oxoquinazolin-3-yl)-1-piperidinyl]carbonyl]-D-tyrosyl]-1-piperazinyl}-3-(trifluoromethyl)-benzoic acid,
(74) 3-{1-[3,5-dibromo-N-[[4-(3,4-dihydro-2(1H)-oxoquinazolin-3-yl)-1-piperidinyl]carbonyl]-D-tyrosyl]-4-piperidinyl}-benzoic acid,
(75) 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoic acid,
(76) 3-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-benzoic acid,
(77) 4-{1-[4-amino-3,5-dibromo-N-[[4-(2-oxo-1,3,4,5-terahydro-1,3-benzodiazepin-3-yl)-1-piperidinyl]carbonyl]-D-phenylalanyl]-4-piperidinyl}-benzoic acid,
(78) 4-{2-[1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl]-ethyl}-benzoic acid,
(79) 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-3-(trifluoromethyl)-benzoic acid,
(80) 3-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-benzoic acid,
(81) ethyl 4-{1-[3-(1-naphthyl)-N-[[4-(2-oxo-1,3,4,5-tetrahydro-1,3-benzodiazepin-3-yl)-1-piperidinyl]carbonyl]-D-alanyl]-4-piperidinyl}-1-piperazineacetate,
(82) 4-{1-[3-(1-naphthyl)-N-[[4-(2-oxo-1,3,4,5-tetrahydro-1,3-benzodiazepin-3-yl)-1-piperidinyl]carbonyl]-D-alanyl]-4-piperidinyl}-1-piperazineacetic acid,
(83) methyl 2-{4-[3,5-dibromo-N-[[4-(3,4-dihydro-2(1H)-oxoquinazolin-3-yl)-1-piperidinyl]carbonyl]-D-tyrosyl]-1-piperazinyl}-5-thiazolecarboxylate,
(84) methyl 2-{4-[3,5-dibromo-N-[[4-(3,4-dihydro-2(1H)-oxoquinazolin-3-yl)-1-piperidinyl]carbonyl]-D-tyrosyl]-1-piperazinyl}-4-thiazolecarboxylate,
(85) 2-{4-[3,5-dibromo-N-[[4-(3,4-dihydro-2(1H)-oxoquinazolin-3-yl)-1-piperidinyl]carbonyl]-D-tyrosyl]-1-piperazinyl}-5-thiazolecarboxylic acid,
(86) 2-{4-[3,5-dibromo-N-[[4-(3,4-dihydro-2(1H)-oxoquinazolin-3-yl)-1-piperidinyl]carbonyl]-D-tyrosyl]-1-piperazinyl}-4-thiazolecarboxylic acid,
(87) methyl 2-{4-[4-amino-3,5-dibromo-N-[[4-(3,4-dihydro-2(1H)-oxoquinazolin-3-yl)-1-piperidinyl]carbonyl]-D-phenylalanyl]-1-piperazinyl}-4-thiazolecarboxylate,
(88) methyl 2-{4-[4-amino-3,5-dibromo-N-[[4-(3,4-dihydro-2(1H)-oxoquinazolin-3-yl)-1-piperidinyl]carbonyl]-D-phenylalanyl]-1-piperazinyl}-5-thiazolecarboxylate,
(89) 2-{4-[4-amino-3,5-dibromo-N-[[4-(3,4-dihydro-2(1H)-oxoquinazolin-3-yl)-1-piperidinyl]carbonyl]-D-phenylalanyl]-1-piperazinyl}-4-thiazolecarboxylic acid,
(90) 2-{4-[4-amino-3,5-dibromo-N-[[4-(3,4-dihydro-2(1H)-oxoquinazolin-3-yl)-1-piperidinyl]carbonyl]-D-phenylalanyl]-1-piperazinyl}-5-thiazolecarboxylic acid,
(91) 4-{4-[4-amino-3,5-dibromo-N-[[4-(3,4-dihydro-2(1H)-oxoquinazolin-3-yl)-1-piperidinyl]carbonyl]-D-phenylalanyl]-1-piperazinyl}-1-piperidineacetic acid,
(92) 4-{4-[4-amino-3,5-dibromo-N-[[4-(2-oxo-1,3,4,5-tetrahydro-1,3-benzodiazepin-3-yl)-1-piperidinyl]carbonyl]-D-phenylalanyl]-1-piperazinyl}-1-piperidineacetic acid,
(93) 1,1-dimethylethyl 4-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-1-piperazineacetate,
(94) 1,1-dimethylethyl 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-1-piperazineacetate,
(95) ethyl 4-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-1-piperazineacetate,
(96) ethyl 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-1-piperazineacetate,
(97) 4-{1-[4-amino-3,5-dibromo-N-[[4-(3,4-dihydro-2(1H)-oxoquinazolin-3-yl)-1-piperidinyl]carbonyl]-D-phenylalanyl]-4-piperidinyl}-1-piperazineacetic acid,
(98) 4-{1-[4-amino-3,5-dibromo-N-[[4-(2-oxo-1,3,4,5-tetrahydro-1,3-benzodiazepin-3-yl)-1-piperidinyl]carbonyl]-D-phenylalanyl]-4-piperidinyl}-1-piperazineacetic acid,
(99) (R,S)-4-{1-[2-[(4-amino-3,5-dibromophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(100) (R,S)-4-{1-[2-[(3,5-dibromo-4-fluorophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(101) (R,S)-4-{1-[4-[4-(3,4-dihydro-2(1H)-oxopyrido[3,4-d]pyrimidin-3-yl)-1-piperidinyl]-2-[(1-naphthyl)methyl]-1,4-dioxobutyl]-4-piperidinyl}-1-piperazineacetic acid,
(102) (R,S)-4-{1-[2-[[3,5-bis-(trifluoromethyl)-phenyl]methyl]-4-[4-(3,4-dihydro-2(1H)-oxoquinazolin-3-yl)-1-piperidinyl]-1,4-dioxobutyl]-4-piperidinyl}-1-piperazineacetic acid,
(103) (R,S)-4-{1-[4-[4-(3,4-dihydro-2(1H)-oxoquinazolin-3-yl)-1-piperidinyl]-2-[(3,4,5-trimethylphenyl)methyl]-1,4-dioxobutyl]-4-piperidinyl}-1-piperazineacetic acid,
(104) (R,S)-4-{1-[2-[(3-bromo-4,5-dimethylphenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(105) (R,S)-4-{1-[4-[4-(3,4-dihydro-2(1H)-oxoquinazolin-3-yl)-1-piperidinyl]-2-[[3-(trifluoromethyl)-phenyl]methyl]-1,4-dioxobutyl]-4-piperidinyl}-1-piperazineacetic acid,
(106) (R,S)-4-{1-[4-[4-(3,4-dihydro-2(1H)-oxoquinazolin-3-yl)-1-piperidinyl]-2-[(4-methoxy-3,5-dimethylphenyl)methyl]-1,4-dioxobutyl]-4-piperidinyl}-1-piperazineacetic acid,
(107) (R,S)-4-{1-[2-[(4-amino-3,5-dichlorophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(108) (R,S)-4-{1-[2-[[2,4-bis-(trifluoromethyl)-phenyl]methyl]-4-[4-(3,4-dihydro-2(1H)-oxoquinazolin-3-yl)-1-piperidinyl]-1,4-dioxobutyl]-4-piperidinyl}-1-piperazineacetic acid,
(109) (R,S)-4-{1-[2-[(3,5-dibromo-4-methylphenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(110) (R,S)-4-{1-[4-[4-(3,4-dihydro-2(1H)-oxoquinazolin-3-yl)-1-piperidinyl]-2-[(3,4,5-tribromophenyl)methyl]-1,4-dioxobutyl]-4-piperidinyl}-1-piperazineacetic acid,
(111) (R,S)-4-{1-[4-[4-(3,4-dihydro-2(1H)-oxoquinazolin-3-yl)-1-piperidinyl]-2-[(3,4-dimethoxyphenyl)methyl]-1,4-dioxobutyl]-4-piperidinyl}-1-piperazineacetic acid,
(112) (R,S)-4-{1-[2-[(3,4-dichlorophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(113) (R,S)-4-{1-[2-[(4-bromo-3,5-dichlorophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(114) (R,S)-4-{1-[4-[4-(3,4-dihydro-2(1H)-oxoquinazolin-3-yl)-1-piperidinyl]-2-[(2-naphthyl)methyl]-1,4-dioxobutyl]-4-piperidinyl}-1-piperazineacetic acid,
(115) (R,S)-4-{1-[2-[(2,3-dihydrobenzo[b]fur-5-yl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(116) (R,S)-4-{1-[4-[4-(3,4-dihydro-2(1H)-oxoquinazolin-3-yl)-1-piperidinyl]-2-[(1,2,3,4-tetrahydro-1-naphthyl)methyl]-1,4-dioxobutyl]-4-piperidinyl}-1-piperazineacetic acid,
(117) (R,S)-4-{1-[2-[(3,5-dibromo-4-methylphenyl)methyl]-4-[4-(3,4-dihydro-2,2-dioxido-2,1,3-benzothiadiazin-3-yl)-1-piperidinyl]-1,4-dioxobutyl]4-piperidinyl}-1-piperazineacetic acid,
(118) (R,S)-4-{1-[2-[(2,3-dichlorophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(119) ethyl(R,S)-4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-piperazineacetate,
(120) (R,S)-4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-piperazineacetic acid,
(121) (R,S)-4-{4-[2-[(3,4-dibromophenyl)methyl]-4-[4-(3,4-dihydro-2(1H)-oxoquinazolin-3-yl)-1-piperidinyl]-1,4-dioxobutyl]-1-piperazinyl}-1-piperidineacetic acid,
(122) methyl 1-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-(S)-pyrrolidine-2-carboxylate,
(123) methyl 1-{1-[3-chloro-N-[[4-(3,4-dihydro-2(1H)-oxoquinazolin-3-yl)-1-piperidinyl]carbonyl]-4-methyl-D,L-phenylalanyl]-4-piperidinyl}-(S)-pyrrolidine-2-carboxylate,
(124) methyl 1-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-(R)-pyrrolidine-2-carboxylate,
(125) methyl 1-{1-[3-chloro-N-[[4-(3,4-dihydro-2(1H)-oxoquinazolin-3-yl)-1-piperidinyl]carbonyl]-4-methyl-D,L-phenylalanyl]-4-piperidinyl}-(R)-pyrrolidine-2-carboxylate,
(126) 1-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-(S)-pyrrolidine-2-carboxylic acid,
(127) 1-{1-[3-chloro-N-[[4-(3,4-dihydro-2(1H)-oxoquinazolin-3-yl)-1-piperidinyl]-carbonyl]-4-methyl-D,L-phenylalanyl]-4-piperidinyl}-(S)-pyrrolidine-2-carboxylic acid,
(128) ethyl 4-{1-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-4-piperidinyl}-1-methyl-2-piperazinecarboxylate,
(129) ethyl 4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylate,
(130) ethyl 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-2-piperazinecarboxylate,
(131) ethyl 4-{1-[2-[(3,5-dibromo-4-methylphenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylate,
(132) ethyl 4-[2-[(3,5-dibromo-4-methylphenyl)methyl]-4-[4-(3,4-dihydro-2(1H)-oxoquinazolin-3-yl)-1-piperidinyl]-1,4-dioxobutyl]-1-(1-methyl-4-piperidinyl)-2-piperazinecarboxylate,
(133) ethyl 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylate,
(134) 4-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylic acid,
(135) 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-2-piperazinecarboxylic acid,
(136) 4-[2-[(3,4-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylic acid,
(137) 4-{1-[2-[(3,5-dibromo-4-methylphenyl)methyl]-4-[4-(3,4-dihydro-2(1H)-oxo-quinazolin-3-yl)-1-piperidinyl]-1,4-dioxobutyl]-4-piperidinyl}-1-methyl-2-piperazinecarboxylic acid,
(138) ethyl 4-[2-[(3,5-dibromo-4-methylphenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylate,
(139) 4-[2-[(3,5-dibromo-4-methylphenyl)methyl]-4-[4-(3,4-dihydro-2(1H)-oxo-quinazolin-3-yl)-1-piperidinyl]-1,4-dioxobutyl]-1-(1-methyl-4-piperidinyl)-2-piperazinecarboxylic acid,
(140) ethyl 4-[2-[(4-amino-3,5-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-2-piperazinecarboxylate,
(141) ethyl 4-[2-[(4-amino-3,5-dibromophenyl)methyl]-4-[4-(3,4-dihydro-2(1H)-oxoquinazolin-3-yl)-1-piperidinyl]-1,4-dioxobutyl]-1-(1-methyl-4-piperidinyl)-3-piperazinecarboxylate,
(142) ethyl 4-{1-[2-[(4-amino-3,5-dibromophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylate,
(143) 4-[2-[(4-amino-3,5-dibromophenyl)methyl]-4-[4-(3,4-dihydro-2(1H)-oxo-quinazolin-3-yl)-1-piperidinyl]-1,4-dioxobutyl]-1-(1-methyl-4-piperidinyl)-2-piperazinecarboxylic acid,
(144) 4-{1-[2-[(4-amino-3,5-dibromophenyl)methyl]-4-[4-(3,4-dihydro-2(1H)-oxo-quinazolin-3-yl)-1-piperidinyl]-1,4-dioxobutyl]-4-piperidinyl}-1-methyl-2-piperazinecarboxylic acid,
(145) 4-[2-[(4-amino-3,5-dibromophenyl)methyl]-4-[4-(3,4-dihydro-2(1H)-oxo-quinazolin-3-yl)-1-piperidinyl]-1,4-dioxobutyl]-1-(1-methyl-4-piperidinyl)-3-piperazinecarboxylic acid,
(146) 4-[2-[(3,5-dibromo-4-methylphenyl)methyl]-4-[4-(3,4-dihydro-2(1H)-oxo-quinazolin-3-yl)-1-piperidinyl]-1,4-dioxobutyl]-1-(1-methyl-4-piperidinyl)-3-piperazinecarboxylic acid,
(147) ethyl 4-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-1-(1-methyl-4-piperidinyl)-2-piperazinecarboxylate,
(148) ethyl 4-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-1-(1-methyl-4-piperidinyl)-3-piperazinecarboxylate,
(149) 4-{1-[4-[4-(3,4-dihydro-2(1H)-oxoquinazolin-3-yl)-1-piperidinyl]-2-[(3,5-di-methyl-4-hydroxyphenyl)methyl]-1,4-dioxobutyl]-4-piperidinyl}-1-methyl-2-piperazinecarboxylic acid,
(150) ethyl 4-{1-[2-[(4-bromo-3,5-dichlorophenyl)methyl]-4-[4-(3,4-dihydro-2(1H)-oxoquinazolin-3-yl)-1-piperidinyl]-1,4-dioxobutyl]-4-piperidinyl}-1-methyl-2-piperazinecarboxylate,
(151) ethyl 1-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylate,
(152) ethyl 1-{1-[2-[(3,5-dibromo-4-methylphenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylate,
(153) ethyl 1-{1-[4-[4-(3,4-dihydro-2(1H)-oxoquinazolin-3-yl)-1-piperidinyl]-2-[(3,5-dimethyl-4-hydroxyphenyl)methyl]-1,4-dioxobutyl]-4-piperidinyl}-4-methyl-2-piperazinecarboxylate,
(154) ethyl 1-{1-[2-[(4-amino-3,5-dibromophenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylate,
(155) ethyl 1-{1-[2-[(4-bromo-3,5-dichlorophenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylate,
(156) 1-{1-[2-[(3,5-dibromo-4-methylphenyl)methyl]-4-[4-(3,4-dihydro-2(1H)-oxo-quinazolin-3-yl)-1-piperidinyl]-1,4-dioxobutyl]-4-piperidinyl}-4-methyl-2-piperazinecarboxylic acid,
(157) 1-{1-[4-[4-(3,4-dihydro-2(1H)-oxoquinazolin-3-yl)-1-piperidinyl]-2-[(3,5-di-methyl-4-hydroxyphenyl)methyl]-1,4-dioxobutyl]-4-piperidinyl}-4-methyl-2-piperazinecarboxylic acid,
(158) 1-{1-[2-[(4-amino-3,5-dibromophenyl)methyl]-4-[4-(3,4-dihydro-2(1H)-oxo-quinazolin-3-yl)-1-piperidinyl]-1,4-dioxobutyl]-4-piperidinyl}-4-methyl-2-piperazinecarboxylic acid,
(159) 1-{1-[2-[(4-bromo-3,5-dichlorophenyl)methyl]-4-[4-(3,4-dihydro-2(1H)-oxo-quinazolin-3-yl)-1-piperidinyl]-1,4-dioxobutyl]-4-piperidinyl}-4-methyl-2-piperazinecarboxylic acid,
(160) 1-{1-[2-[(3,4-dibromophenyl)methyl]-4-[4-(3,4-dihydro-2(1H)-oxoquinazolin-3-yl)-1-piperidinyl]-1,4-dioxobutyl]-4-piperidinyl}-4-methyl-2-piperazinecarboxylic acid,
(161) ethyl 4-{1-[3,4-dibromo-N-[[4-(3,4-dihydro-2(1H)-oxoquinazolin-3-yl)-1-piperidinyl]carbonyl]-phenylalanyl]-4-piperidinyl}-1-methyl-2-piperazinecarboxylate,
(162) 4-{1-[2-[(4-bromo-3,5-dichlorophenyl)methyl]-4-[4-(3,4-dihydro-2(1H)-oxo-quinazolin-3-yl)-1-piperidinyl]-1,4-dioxobutyl]-4-piperidinyl}-1-methyl-2-piperazinecarboxylic acid,
(163) methyl 1′-[3,5-dibromo-N-[[4-(3,4-dihydro-2(1H)-oxoquinazolin-3-yl)-1-piperidinyl]carbonyl]-phenylalanyl]-[1,4′]bipiperidinyl-4-acetate,
(164) 1′-[3,5-dibromo-N-[[4-(3,4-dihydro-2(1H)-oxoquinazolin-3-yl)-1-piperidinyl]-carbonyl]-phenylalanyl]-[1,4′] bipiperidinyl-4-acetic acid,
(165) ethyl 4-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-1-methyl-2-piperazinecarboxylate,
(166) ethyl 1-{1-[3,5-dibromo-N-[[4-(3,4-dihydro-2(1H)-oxoquinazolin-3-yl)-1-piperidinyl]carbonyl]-4-methyl-D,L-phenylalanyl]-4-piperidinyl}-4-methyl-2-piperazinecarboxylate
or the salts thereof.

6. Physiologically acceptable salts of the compounds according to one of claims 1 to 5 with inorganic or organic acids or bases inorganic or organic acids or bases.

7. Pharmaceutical compositions containing a compound according to one of claims 1 to 5, optionally together with one or more inert carriers and/or diluents.

8. Pharmaceutical compositions containing a physiologically acceptable salt according to claim 6, optimally together with one or more inert carriers and/or diluents.

9. A method for the acute or prophylactic treatment of headaches comprising administering a pharmaceutical composition according to one of claims 1 to 5.

10. A method for the acute or prophylactic treatment of headaches comprising administering a pharmaceutical composition according to claim 6.

11. A method for treating a disease or syndrome selected from the group consisting of: non-insulin-dependent diabetes mellitus, cardiovascular diseases, morphine tolerance, skin diseases, inflammatory diseases, allergic rhinitis, asthma, diseases accompanied by excessive vasodilation and resultant reduced circulation of the blood, and pain comprising administering a pharmaceutical composition according to one of claims 1 to 5.

12. A method for treating a disease or syndrome selected from the group consisting of: non-insulin-dependent diabetes mellitus, cardiovascular diseases, morphine tolerance, skin diseases, inflammatory diseases, allergic rhinitis, asthma, diseases accompanied by excessive vasodilation and resultant reduced circulation of the blood, and pain comprising administering a pharmaceutical composition according to claim 6.

13. A method for the acute or prophylactic treatment of menopausal hot flashes in estrogen-deficient women comprising administering a pharmaceutical composition according to one of claims 1 to 5.

14. A method for the acute or prophylactic treatment of menopausal hot flashes in estrogen-deficient women comprising administering a pharmaceutical composition according to claim 6.
